SEQUENCE LISTING (288 sequences)

SEQ ID NO: 1 - protein, 118 aa, Mus musculus
MSQGPPAVSVLQRSVDAPGNQPQSPKDDDRKVRRREKNRVAAQRSRKKQTQKADKLHEEHESLEQENSVLRREISKLKEELRHLSEVLKEHEKMCPLLLCPMNFVQLRSDPVASCLPR

SEQ ID NO: 2 - protein, 125 aa, Mus musculus
MPHSSDSSDSSFSRSPPPGKQDSSDDVRKVQRREKNRIAAQKSRQRQTQKADTLHLESEDLEKQNAALRKEIKQLTEELKYFTSVLSSHEPLCSVLASGTPSPPEVVYSAHAFHQPHISSPRFQP

SEQ ID NO: 3 - DNA, 34 bp, Mus musculus
attactcgagtgaaacaaacaggcagtcgcagtg

SEQ ID NO: 4 - DNA, 34 bp, Mus musculus
attactcgagcctactacctttcagggctactgc

SEQ ID NO: 5 - DNA, 36 bp, Mus musculus
attagtcgacgcattcttcatggtccttagccttgg

SEQ ID NO: 6 - DNA, 33 bp, Mus musculus
attagtcgaccagagaatgagaaatgttggagg

SEQ ID NO: 7 - DNA, 23 bp, Mus musculus
caactgggtctgagtcaagaggt

SEQ ID NO: 8 - DNA, 24 bp, Mus musculus
cgtagccgctgattgttttagaac

SEQ ID NO: 9 - DNA, 24 bp, Mus musculus
acagcttgaacttcagagccctcc

SEQ ID NO: 10 - DNA, 25 bp, Mus musculus
cacatttaagtcacaataacactgc

SEQ ID NO: 11 - DNA, 21 bp, Mus musculus
ggaagattagaaccatgcctc

SEQ ID NO: 12 - DNA, 19 bp, Mus musculus
agaaggtcagggctggaag

SEQ ID NO: 13 - DNA, 44 bp, Mus musculus
ggactacaaagacgatgacgacaagcctcacagctccgacagca

SEQ ID NO: 14 - DNA, 44 bp, Mus musculus; identical to SEQ ID NO: 13

SEQ ID NO: 15 - DNA, 17 bp, Mus musculus
cgctgagagggcttcac

SEQ ID NO: 16 - DNA, 21 bp, Mus musculus
gcaggagtaggccacattaca

SEQ ID NO: 17 - DNA, 24 bp, Mus musculus
atcctgaacttctatcagctccac

SEQ ID NO: 18 - DNA, 25 bp, Mus musculus
gcatttagctatgtgcttctgtttc

SEQ ID NO: 19 - DNA, 20 bp, Mus musculus
catgcaggaggtggtacctt

SEQ ID NO: 20 - DNA, 20 bp, Mus musculus
cagacgcaagcatttctcag

SEQ ID NO: 21 - DNA, 20 bp, Mus musculus
tctccctgcgctctccgcac

SEQ ID NO: 22 - DNA, 19 bp, Mus musculus
tccacagatcttgcatgga

SEQ ID NO: 23 - DNA, 18 bp, Mus musculus
gcccaacaagctagaaag

SEQ ID NO: 24 - DNA, 20 bp, Mus musculus
tctctgagggtctggaaact

SEQ ID NO: 25 - DNA, 17 bp, Mus musculus
agcctaagatgagcgcc

SEQ ID NO: 26 - DNA, 20 bp, Mus musculus
ttactaggcagatggccaca

SEQ ID NO: 27 - DNA, 25 bp, Mus musculus
ctcgaggtgtgctgtcctgggctac

SEQ ID NO: 28 - DNA, 24 bp, Mus musculus
ctcgaggggagacgggtcagaggg

SEQ ID NO: 29 - DNA, 25 bp, Mus musculus
aagcttgaacaggagctatcggtcc

SEQ ID NO: 30 - DNA, 27 bp, Mus musculus
aagcttgaggtggatgaagagtagtgc

SEQ ID NO: 31 - DNA, 28 bp, Mus musculus
ggttctgtgctgacctcatttgaggatg

SEQ ID NO: 32 - DNA, 27 bp, Mus musculus
aaaagactgggtgaaatttagttaaag

SEQ ID NO: 33 - DNA, 20 bp, Mus musculus
aaatgtgagccccagatcga

SEQ ID NO: 34 - DNA, 21 bp, Mus musculus
gggacattttttccaccatga

SEQ ID NO: 35 - DNA, 24 bp, Mus musculus
ctgctgctgtcccaggcacagttg

SEQ ID NO: 36 - DNA, 19 bp, Mus musculus
ttgtcccctggctgttcct

SEQ ID NO: 37 - DNA, 20 bp, Mus musculus
gggctccccaaaaattcaca

SEQ ID NO: 38 - DNA, 24 bp, Mus musculus
ccttatccagctgtctttttctct

SEQ ID NO: 39 - DNA, 23 bp, Mus musculus
gtccctctgttgtttccaaggat

SEQ ID NO: 40 - DNA, 20 bp, Mus musculus
gccatttcagccactgtgaa

SEQ ID NO: 41 - DNA, 30 bp, Mus musculus
tcattgagtccttccagcagagatttcagg

SEQ ID NO: 42 - DNA, 21 bp, Mus musculus
tggcaaatgttttgtcaacca

SEQ ID NO: 43 - DNA, 20 bp, Mus musculus
catgcagcctctgcttgaga

SEQ ID NO: 44 - DNA, 24 bp, Mus musculus
ttcctcgattgctgtctactcatc

SEQ ID NO: 45 - DNA, 25 bp, Mus musculus
cgatacttttcagtgacatccgttt

SEQ ID NO: 46 - DNA, 26 bp, Mus musculus
tgctgacttcatctgatacccttaga

SEQ ID NO: 47 - DNA, 30 bp, Mus musculus
acttgaaacccagtcagttgctgaccttga

SEQ ID NO: 48 - DNA, 21 bp, Mus musculus
gaacttctgcccttcccatct

SEQ ID NO: 49 - DNA, 20 bp, Mus musculus
cagcacagaaccaccccttt

SEQ ID NO: 50 - DNA, 25 bp, Mus musculus
ccttcgagacagatgttgcccgtca

SEQ ID NO: 51 - DNA, 22 bp, Mus musculus
atttagggcacaggtgacatga

SEQ ID NO: 52 - DNA, 20 bp, Mus musculus
ccacttccccgacctcacta

SEQ ID NO: 53 - DNA, 26 bp, Mus musculus
tggttctcaaagcataaacctcattc

SEQ ID NO: 54 - DNA, 21 bp, Mus musculus
caaatccgtgtgccttctgtt

SEQ ID NO: 55 - DNA, 21 bp, Mus musculus
aggttgacttcgtccctgtga

SEQ ID NO: 56 - DNA, 29 bp, Mus musculus
ctgcagtgaggaagatgtttccaatgagg

SEQ ID NO: 57 - DNA, 21 bp, Mus musculus
gtggcctacttcaggcagatg

SEQ ID NO: 58 - DNA, 20 bp, Mus musculus
ggagccgatgagaagcattc

SEQ ID NO: 59 - DNA, 21 bp, Mus musculus
tgagaagccagcgtcgggtcc

SEQ ID NO: 60 - DNA, 26 bp, Mus musculus
agataatgtatcacacagccctgaag

SEQ ID NO: 61 - DNA, 24 bp, Mus musculus
catggttgtgaagttggtgagatg

SEQ ID NO: 62 - DNA, 23 bp, Mus musculus
agccagtgccttaatccattggg

SEQ ID NO: 63 - DNA, 19 bp, Mus musculus
actgcatgacccgaaagca

SEQ ID NO: 64 - DNA, 21 bp, Mus musculus
tttaattcccccacaaagcaa

SEQ ID NO: 65 - DNA, 26 bp, Mus musculus
aacccacacgcagagcatgacaagag

SEQ ID NO: 66 - DNA, 19 bp, Mus musculus
ttcccttttctgccttgca

SEQ ID NO: 67 - DNA, 23 bp, Mus musculus
tgtgtaacacgcagagtggaatg

SEQ ID NO: 68 - DNA, 20 bp, Mus musculus
acgaagcacagggctgggcc

SEQ ID NO: 69 - protein, 125 aa, Homo sapiens
MPHSSDSSDSSFSRSPPPGKQDSSDDVRRVQRREKNRIAAQKSRQRQTQKADTLHLESEDLEKQNAALRKEIKQLTEELKYFTSVLNSHEPLCSVLAASTPSPPEVVYSAHAFHQPHVSSPRFQP

SEQ ID NO: 70 - DNA, 28 bp, Mus musculus
gcacccagcaccagctgatcaggacgcg

SEQ ID NO: 71 - DNA, 28 bp, Mus musculus
gtttgcgcgtcctgatcagctggtgctg

SEQ ID NO: 72 - DNA, 28 bp, Mus musculus
acgaggcacaagtgcacccagcaccagc

SEQ ID NO: 73 - DNA, 28 bp, Mus musculus
gatcagctggtgctgggtgcacttgtgc

SEQ ID NO: 74 - DNA, 28 bp, Mus musculus
gcactactcttcatccacctcacacgag

SEQ ID NO: 75 - DNA, 28 bp, Mus musculus
tgtgcctcgtgtgaggtggatgaagagt

SEQ ID NO: 76 - DNA, 28 bp, Mus musculus
aaagagagaaaggagcactactcttcat

SEQ ID NO: 77 - DNA, 28 bp, Mus musculus
ggtggatgaagagtagtgctcctttctc

SEQ ID NO: 78 - DNA, 28 bp, Mus musculus
gtagtaaaaccgtataaaaagagagaaa

SEQ ID NO: 79 - DNA, 28 bp, Mus musculus
gctcctttctctctttttatacggtttt

SEQ ID NO: 80 - DNA, 27 bp, Mus musculus
acgtaagtgaccacagaggtagtaaaa

SEQ ID NO: 81 - DNA, 27 bp, Mus musculus
tacggttttactacctctgtggtcact

SEQ ID NO: 82 - DNA, 30 bp, Mus musculus
gtcaccccccaacccactcttgacgtaagt

SEQ ID NO: 83 - DNA, 30 bp, Mus musculus
tggtcacttacgtcaagagtgggttggggg

SEQ ID NO: 84 - DNA, 28 bp, Mus musculus
gaatctttactcaaatggtgtcaccccc

SEQ ID NO: 85 - DNA, 28 bp, Mus musculus
ggttggggggtgacaccatttgagtaaa

SEQ ID NO: 86 - DNA, 28 bp, Mus musculus
tttgaggatggaatctttactcaaatgg

SEQ ID NO: 87 - DNA, 28 bp, Mus musculus
tgacaccatttgagtaaagattccatcc

SEQ ID NO: 88 - DNA, 28 bp, Mus musculus
ggttctgtgctgacctcatttgaggatg

SEQ ID NO: 89 - DNA, 28 bp, Mus musculus
gattccatcctcaaatgaggtcagcaca

SEQ ID NO: 90 - DNA, 28 bp, Mus musculus
gcccgtcataaaggggtggttctgtgct

SEQ ID NO: 91 - DNA, 28 bp, Mus musculus
aggtcagcacagaaccacccctttatga

SEQ ID NO: 92 - DNA, 28 bp, Mus musculus
agacagatgttgcccgtcataaaggggt

SEQ ID NO: 93 - DNA, 28 bp, Mus musculus
gaaccacccctttatgacgggcaacatc

SEQ ID NO: 94 - DNA, 28 bp, Mus musculus
gcccttcccatctaccttcgagacagat

SEQ ID NO: 95 - DNA, 28 bp, Mus musculus
gcaacatctgtctcgaaggtagatggga

SEQ ID NO: 96 - DNA, 29 bp, Mus musculus
gcatagtgaacttctgcccttcccatcta

SEQ ID NO: 97 - DNA, 29 bp, Mus musculus
gaaggtagatgggaagggcagaagttcac

SEQ ID NO: 98 - DNA, 28 bp, Mus musculus
gaagtcatgcttctttgcatagtgaact

SEQ ID NO: 99 - DNA, 28 bp, Mus musculus
gcagaagttcactatgcaaagaagcatg

SEQ ID NO: 100 - DNA, 28 bp, Mus musculus
ctgttcagctcccaagaagtcatgcttc

SEQ ID NO: 101 - DNA, 28 bp, Mus musculus
gcaaagaagcatgacttcttgggagctg

SEQ ID NO: 102 - DNA, 29 bp, Mus musculus
ctgaatcacagcaaagcatctctgttcag

SEQ ID NO: 103 - DNA, 29 bp, Mus musculus
gggagctgaacagagatgctttgctgtga

SEQ ID NO: 104 - DNA, 30 bp, Mus musculus
gtccatacacacatgatactgaatcacagc

SEQ ID NO: 105 - DNA, 30 bp, Mus musculus
gctttgctgtgattcagtatcatgtgtgta

SEQ ID NO: 106 - DNA, 28 bp, Mus musculus
gcagcttcagatatgtccatacacacat

SEQ ID NO: 107 - DNA, 28 bp, Mus musculus
gtatcatgtgtgtatggacatatctgaa

SEQ ID NO: 108 - DNA, 28 bp, Mus musculus
gagcccagctctgcagcagcttcagata

SEQ ID NO: 109 - DNA, 28 bp, Mus musculus
ggacatatctgaagctgctgcagagctg

SEQ ID NO: 110 - DNA, 29 bp, Mus musculus
gactcacaaaccattactatggagcccag

SEQ ID NO: 111 - DNA, 29 bp, Mus musculus
cagagctgggctccatagtaatggtttgt

SEQ ID NO: 112 - DNA, 28 bp, Mus musculus
gagactgtcaagagactcacaaaccatt

SEQ ID NO: 113 - DNA, 28 bp, Mus musculus
atagtaatggtttgtgagtctcttgaca

SEQ ID NO: 114 - DNA, 28 bp, Mus musculus
aaagtgtgtgtcactaggagactgtcaa

SEQ ID NO: 115 - DNA, 28 bp, Mus musculus
gtctcttgacagtctcctagtgacacac

SEQ ID NO: 116 - DNA, 28 bp, Mus musculus
gatcaagtcaaaattcaaagtgtgtgtc

SEQ ID NO: 117 - DNA, 28 bp, Mus musculus
ctagtgacacacactttgaattttgact

SEQ ID NO: 118 - DNA, 28 bp, Mus musculus
ggtagaaaagtgagaaagatcaagtcaa

SEQ ID NO: 119 - DNA, 28 bp, Mus musculus
gaattttgacttgatctttctcactttt

SEQ ID NO: 120 - DNA, 28 bp, Mus musculus
gccagggaatttggtagaaaagtgagaa

SEQ ID NO: 121 - DNA, 28 bp, Mus musculus
gatctttctcacttttctaccaaattcc

SEQ ID NO: 122 - DNA, 28 bp, Mus musculus
gggcaagggatgctctctagccagggaa

SEQ ID NO: 123 - DNA, 28 bp, Mus musculus
gcaaattccctggctagagagcatccct

SEQ ID NO: 124 - DNA, 28 bp, Mus musculus
gtgggtttctttgggcaagggatgctct

SEQ ID NO: 125 - DNA, 28 bp, Mus musculus
gctagagagcatcccttgcccaaagaaa

SEQ ID NO: 126 - DNA, 28 bp, Mus musculus
gtttacatactaagacattgagtgggtt

SEQ ID NO: 127 - DNA, 28 bp, Mus musculus
aaagaaacccactcaatgtcttagtatg

SEQ ID NO: 128 - DNA, 28 bp, Mus musculus
gtcatcagctcctggagactcagttctg

SEQ ID NO: 129 - DNA, 28 bp, Mus musculus
gccaccagaactgagtctccaggagctg

SEQ ID NO: 130 - DNA, 29 bp, Mus musculus
gtgagaaccagaccaaggccctgtcatca

SEQ ID NO: 131 - DNA, 29 bp, Mus musculus
ggagctgatgacagggccttggtctggtt

SEQ ID NO: 132 - DNA, 28 bp, Mus musculus
agtcaggttgaagtgagaaccagaccaa

SEQ ID NO: 133 - DNA, 28 bp, Mus musculus
gggccttggtctggttctcacttcaacc

SEQ ID NO: 134 - DNA, 26 bp, Mus musculus
tagcgacaacctgtgcacagtcaggt

SEQ ID NO: 135 - DNA, 26 bp, Mus musculus
gttcaacctgactgtgcacaggttgt

SEQ ID NO: 136 - DNA, 28 bp, Mus musculus
gatgaataaataggtagccgtagcgaca

SEQ ID NO: 137 - DNA, 28 bp, Mus musculus
caggttgtcgctacggctacctatttat

SEQ ID NO: 138 - DNA, 28 bp, Mus musculus
ggcctcttcttgagggatgaataaatag

SEQ ID NO: 139 - DNA, 28 bp, Mus musculus
gctacctatttattcatccctcaagaag

SEQ ID NO: 140 - DNA, 28 bp, Mus musculus
ctgcaatgggagggcttggcctcttctt

SEQ ID NO: 141 - DNA, 28 bp, Mus musculus
gcctcaagaagaggccaagccctcccat

SEQ ID NO: 142 - DNA, 28 bp, Mus musculus
aaagatttccaggctgcaatgggagggc

SEQ ID NO: 143 - DNA, 28 bp, Mus musculus
gccaagccctcccattgcagcctggaaa

SEQ ID NO: 144 - DNA, 28 bp, Mus musculus
gttactcacactcatccactatacaaag

SEQ ID NO: 145 - DNA, 28 bp, Mus musculus
gaaatctttgtatagtggatgagtgtga

SEQ ID NO: 146 - DNA, 28 bp, Mus musculus
gaaaaacgagttactcacactcatccac

SEQ ID NO: 147 - DNA, 28 bp, Mus musculus
gtatagtggatgagtgtgagtaactcgt

SEQ ID NO: 148 - DNA, 28 bp, Mus musculus
cacgtacacctagccaatggaaaagaaa

SEQ ID NO: 149 - DNA, 28 bp, Mus musculus
tcgtttttcttttccattggctaggtgt

SEQ ID NO: 150 - DNA, 28 bp, Mus musculus
tgcccccacacgcacacgtacacctagc

SEQ ID NO: 151 - DNA, 28 bp, Mus musculus
cattggctaggtgtacgtgtgcgtgtgg

SEQ ID NO: 152 - DNA, 28 bp, Mus musculus
tgtggactctatccatccctgcccccac

SEQ ID NO: 153 - DNA, 28 bp, Mus musculus
tgcgtgtgggggcagggatggatagagt

SEQ ID NO: 154 - DNA, 28 bp, Mus musculus
gatggggcacattttgtggactctatcc

SEQ ID NO: 155 - DNA, 28 bp, Mus musculus
gggatggatagagtccacaaaatgtgcc

SEQ ID NO: 156 - DNA, 28 bp, Mus musculus
gtctaagatgcagatggggcacattttg

SEQ ID NO: 157 - DNA, 28 bp, Mus musculus
gtccacaaaatgtgccccatctgcatct

SEQ ID NO: 158 - DNA, 28 bp, Mus musculus
gtctctttttcctgtctaagatgcagat

SEQ ID NO: 159 - DNA, 28 bp, Mus musculus
gccccatctgcatcttagacaggaaaaa

SEQ ID NO: 160 - DNA, 28 bp, Mus musculus
gctgaaaactggaattcacccatgtgtc

SEQ ID NO: 161 - DNA, 28 bp, Mus musculus
aaagagacacatgggtgaattccagttt

SEQ ID NO: 162 - DNA, 28 bp, Mus musculus
cttggtgaatgctgaaaactggaattca

SEQ ID NO: 163 - DNA, 28 bp, Mus musculus
atgggtgaattccagttttcagcattca

SEQ ID NO: 164 - DNA, 28 bp, Mus musculus
gacacacacacacacacacaccttggtg

SEQ ID NO: 165 - DNA, 29 bp, Mus musculus
gcattcaccaaggtgtgtgtgtgtgtgtg

SEQ ID NO: 166 - DNA, 28 bp, Mus musculus
gccacacacacacacacacacacacaca

SEQ ID NO: 167 - DNA, 28 bp, Mus musculus
gtgtgtgtgtgtgtgtgtgtgtgtgtgt

SEQ ID NO: 168 - DNA, 28 bp, Mus musculus
gaaatctgacggtgcctcctgtgccaca

SEQ ID NO: 169 - DNA, 28 bp, Mus musculus
gtgtgtgtggcacaggaggcaccgtcag

SEQ ID NO: 170 - DNA, 28 bp, Mus musculus
gtttacttctctgaaatctgacggtgcc

SEQ ID NO: 171 - DNA, 28 bp, Mus musculus
caggaggcaccgtcagatttcagagaag

SEQ ID NO: 172 - DNA, 28 bp, Mus musculus
gatcaaagtgtttgtgtttacttctctg

SEQ ID NO: 173 - DNA, 28 bp, Mus musculus
gatttcagagaagtaaacacaaacactt

SEQ ID NO: 174 - DNA, 28 bp, Mus musculus
tgcagagcaaaagatcaaagtgtttgtg

SEQ ID NO: 175 - DNA, 28 bp, Mus musculus
gtaaacacaaacactttgatcttttgct

SEQ ID NO: 176 - DNA, 28 bp, Mus musculus
gacaaaccaggtgaggtgccagggatgc

SEQ ID NO: 177 - DNA, 28 bp, Mus musculus
gctctgcatccctggcacctcacctggt

SEQ ID NO: 178 - DNA, 30 bp, Mus musculus
gcctttatgactgtcagacaaaccaggtga

SEQ ID NO: 179 - DNA, 30 bp, Mus musculus
gcacctcacctggtttgtctgacagtcata

SEQ ID NO: 180 - DNA, 28 bp, Mus musculus
gtcattgcagaagtgcctttatgactgt

SEQ ID NO: 181 - DNA, 28 bp, Mus musculus
gtctgacagtcataaaggcacttctgca

SEQ ID NO: 182 - DNA, 28 bp, Mus musculus
gccatgccgctgctttactcattgcaga

SEQ ID NO: 183 - DNA, 28 bp, Mus musculus
gcacttctgcaatgagtaaagcagcggc

SEQ ID NO: 184 - DNA, 28 bp, Mus musculus
aaagttccaataaaggccatgccgctgc

SEQ ID NO: 185 - DNA, 28 bp, Mus musculus
gtaaagcagcggcatggcctttattgga

SEQ ID NO: 186 - DNA, 28 bp, Mus musculus
agtcatcaccccataaaaagttccaata

SEQ ID NO: 187 - DNA, 28 bp, Mus musculus
gcctttattggaactttttatggggtga

SEQ ID NO: 188 - DNA, 28 bp, Mus musculus
ggttcagtcaaaaagcatagtcatcacc

SEQ ID NO: 189 - DNA, 28 bp, Mus musculus
tatggggtgatgactatgctttttgact

SEQ ID NO: 190 - DNA, 28 bp, Mus musculus
aatggagtacaggatggttcagtcaaaa

SEQ ID NO: 191 - DNA, 28 bp, Mus musculus
atgctttttgactgaaccatcctgtact

SEQ ID NO: 192 - DNA, 28 bp, Mus musculus
gtaacctcttccatcattgcaatggagt

SEQ ID NO: 193 - DNA, 28 bp, Mus musculus
cctgtactccattgcaatgatggaagag

SEQ ID NO: 194 - DNA, 27 bp, Mus musculus
gcccatcatttaattcttcctaagaag

SEQ ID NO: 195 - DNA, 27 bp, Mus musculus
ggttacttcttaggaagaattaaatga

SEQ ID NO: 196 - DNA, 28 bp, Mus musculus
aggttagaaaactagcccatcatttaat

SEQ ID NO: 197 - DNA, 28 bp, Mus musculus
gaagaattaaatgatgggctagttttct

SEQ ID NO: 198 - DNA, 28 bp, Mus musculus
aggatctaaaatactcttgctaggttag

SEQ ID NO: 199 - DNA, 28 bp, Mus musculus
gttttctaacctagcaagagtattttag

SEQ ID NO: 200 - DNA, 28 bp, Mus musculus
gcacccttacaaaaagataaggatctaa

SEQ ID NO: 201 - DNA, 28 bp, Mus musculus
gtattttagatccttatctttttgtaag

SEQ ID NO: 202 - DNA, 28 bp, Mus musculus
tggaagcaaatcctattttaacaccctt

SEQ ID NO: 203 - DNA, 28 bp, Mus musculus
tttgtaagggtgttaaaataggatttgc

SEQ ID NO: 204 - DNA, 29 bp, Mus musculus
gctatttaaagatacactggtgaaaattg

SEQ ID NO: 205 - DNA, 29 bp, Mus musculus
gcttccaattttcaccagtgtatctttaa

SEQ ID NO: 206 - DNA, 28 bp, Mus musculus
aggcaccattagtgctatttaaagatac

SEQ ID NO: 207 - DNA, 28 bp, Mus musculus
ccagtgtatctttaaatagcactaatgg

SEQ ID NO: 208 - DNA, 28 bp, Mus musculus
gttacataaagtgtcaggaggcaccatt

SEQ ID NO: 209 - DNA, 28 bp, Mus musculus
gcactaatggtgcctcctgacactttat

SEQ ID NO: 210 - DNA, 28 bp, Mus musculus
gtatttacaatccatattgttacataaa

SEQ ID NO: 211 - DNA, 28 bp, Mus musculus
gacactttatgtaacaatatggattgta

SEQ ID NO: 212 - DNA, 28 bp, Mus musculus
agttcatcaaaactgtttattgtattta

SEQ ID NO: 213 - DNA, 28 bp, Mus musculus
gattgtaaatacaataaacagttttgat

SEQ ID NO: 214 - DNA, 28 bp, Mus musculus
gagcacgctgtctacttagttcatcaaa

SEQ ID NO: 215 - DNA, 28 bp, Mus musculus
agttatcaactgttgacacttgtgcgat

SEQ ID NO: 216 - DNA, 28 bp, Mus musculus
cagagatcgcacaagtgtcaacagttga

SEQ ID NO: 217 - DNA, 28 bp, Mus musculus
acaggctctcctctcagttatcaactgt

SEQ ID NO: 218 - DNA, 28 bp, Mus musculus
tgtcaacagttgataactgagaggagag

SEQ ID NO: 219 - DNA, 28 bp, Mus musculus
ttgccttttgctctctcactaacaggct

SEQ ID NO: 220 - DNA, 28 bp, Mus musculus
aggagagcctgttagtgagagagcaaaa

SEQ ID NO: 221 - DNA, 28 bp, Mus musculus
tgctcccctgatgtttttgccttttgct

SEQ ID NO: 222 - DNA, 28 bp, Mus musculus
gagagagcaaaaggcaaaaacatcaggg

SEQ ID NO: 223 - DNA, 28 bp, Mus musculus
gtaccatgctacccgacgaacatgctcc

SEQ ID NO: 224 - DNA, 28 bp, Mus musculus
tcaggggagcatgttcgtcgggtagcat

SEQ ID NO: 225 - DNA, 28 bp, Mus musculus
gacaatcatctgcttggtaccatgctac

SEQ ID NO: 226 - DNA, 28 bp, Mus musculus
gtcgggtagcatggtaccaagcagatga

SEQ ID NO: 227 - DNA, 28 bp, Mus musculus
aggtaagcactcagacctctacagacaa

SEQ ID NO: 228 - DNA, 28 bp, Mus musculus
gatgattgtctgtagaggtctgagtgct

SEQ ID NO: 229 - DNA, 28 bp, Mus musculus
agagacacctaaacaggtaagcactcag

SEQ ID NO: 230 - DNA, 28 bp, Mus musculus
gaggtctgagtgcttacctgtttaggtg

SEQ ID NO: 231 - DNA, 28 bp, Mus musculus
tctgcctctcccatcacaagcagagaca

SEQ ID NO: 232 - DNA, 28 bp, Mus musculus
ttaggtgtctctgcttgtgatgggagag

SEQ ID NO: 233 - DNA, 28 bp, Mus musculus
aaaagcagcaacttctgcctctcccatc

SEQ ID NO: 234 - DNA, 28 bp, Mus musculus
cttgtgatgggagaggcagaagttgctg

SEQ ID NO: 235 - DNA, 28 bp, Mus musculus
cctggtgtcccgatggctataaaagcag

SEQ ID NO: 236 - DNA, 28 bp, Mus musculus
agttgctgcttttatagccatcgggaca

SEQ ID NO: 237 - DNA, 28 bp, Mus musculus
gtcacaataccaaaaaaaccctggtgtc

SEQ ID NO: 238 - DNA, 28 bp, Mus musculus
atcgggacaccagggtttttttggtatt

SEQ ID NO: 239 - DNA, 28 bp, Mus musculus
aatgtctgatgtcatatcattcacaata

SEQ ID NO: 240 - DNA, 28 bp, Mus musculus
tttggtattgtgaatgatatgacatcag

SEQ ID NO: 241 - DNA, 28 bp, Mus musculus
gactggaaattagataatgtctgatgtc

SEQ ID NO: 242 - DNA, 28 bp, Mus musculus
gatatgacatcagacattatctaatttc

SEQ ID NO: 243 - DNA, 28 bp, Mus musculus
gtggttaggtacttctcagaagacagga

SEQ ID NO: 244 - DNA, 28 bp, Mus musculus
tccagtcctgtcttctgagaagtaccta

SEQ ID NO: 245 - DNA, 28 bp, Mus musculus
tggcctcctatggtggttaggtacttct

SEQ ID NO: 246 - DNA, 28 bp, Mus musculus
ttctgagaagtacctaaccaccatagga

SEQ ID NO: 247 - DNA, 28 bp, Mus musculus
ggaaggcttggaggtggtgtcttgtggc

SEQ ID NO: 248 - DNA, 28 bp, Mus musculus
aggaggccacaagacaccacctccaagc

SEQ ID NO: 249 - DNA, 28 bp, Mus musculus
gctctcaaggtgggaaggcttggaggtg

SEQ ID NO: 250 - DNA, 28 bp, Mus musculus
gacaccacctccaagccttcccaccttg

SEQ ID NO: 251 - DNA, 28 bp, Mus musculus
gtgacgttttagggaagacttcccatct

SEQ ID NO: 252 - DNA, 28 bp, Mus musculus
ttgagagatgggaagtcttccctaaaac

SEQ ID NO: 253 - DNA, 28 bp, Mus musculus
tgttggccctcaccgtgacgttttaggg

SEQ ID NO: 254 - DNA, 28 bp, Mus musculus
gtcttccctaaaacgtcacggtgagggc

SEQ ID NO: 255 - DNA, 28 bp, Mus musculus
ctgggatttgtgtgcaaaagcaccttgt

SEQ ID NO: 256 - DNA, 28 bp, Mus musculus
ggccaacaaggtgcttttgcacacaaat

SEQ ID NO: 257 - DNA, 28 bp, Mus musculus
gtgtttagaagatttctgggatttgtgt

SEQ ID NO: 258 - DNA, 28 bp, Mus musculus
tttgcacacaaatcccagaaatcttcta

SEQ ID NO: 259 - DNA, 28 bp, Mus musculus
aatagctacgggagatcaaaggctgctc

SEQ ID NO: 260 - DNA, 28 bp, Mus musculus
gagtagagcagcctttgatctcccgtag

SEQ ID NO: 261 - DNA, 28 bp, Mus musculus
ccgtgaccaaaacgctgactcaatagct

SEQ ID NO: 262 - DNA, 28 bp, Mus musculus
cccgtagctattgagtcagcgttttggt

SEQ ID NO: 263 - DNA, 29 bp, Mus musculus
gaaaatgagtccgtgaccaaaacgctgac

SEQ ID NO: 264 - DNA, 29 bp, Mus musculus
attgagtcagcgttttggtcacggactca

SEQ ID NO: 265 - DNA, 28 bp, Mus musculus
gttggtgggaaaatgagtccgtgaccaa

SEQ ID NO: 266 - DNA, 28 bp, Mus musculus
gcgttttggtcacggactcattttccca

SEQ ID NO: 267 - DNA, 30 bp, Mus musculus
tgaagttggtgggaaaatgagtccgtgacc

SEQ ID NO: 268 - DNA, 30 bp, Mus musculus
gttttggtcacggactcattttcccaccaa

SEQ ID NO: 269 - DNA, 30 bp, Mus musculus
gaatctatgaagttggtgggaaaatgagtc

SEQ ID NO: 270 - DNA, 30 bp, Mus musculus
tcacggactcattttcccaccaacttcata

SEQ ID NO: 271 - DNA, 28 bp, Mus musculus
taaagagataagaatctatgaagttggt

SEQ ID NO: 272 - DNA, 28 bp, Mus musculus
gtcccaccaacttcatagattcttatct

SEQ ID NO: 273 - DNA, 28 bp, Mus musculus
gtatttctggtcacttctaaagagataa

SEQ ID NO: 274 - DNA, 28 bp, Mus musculus
gattcttatctctttagaagtgaccaga

SEQ ID NO: 275 - DNA, 28 bp, Mus musculus
gaatataggacacgggtcttttatttct

SEQ ID NO: 276 - DNA, 28 bp, Mus musculus
tgaccagaaataaaagacccgtgtccta

SEQ ID NO: 277 - DNA, 28 bp, Mus musculus
gcttatttcaaagcacagaatataggac

SEQ ID NO: 278 - DNA, 28 bp, Mus musculus
cccgtgtcctatattctgtgctttgaaa

SEQ ID NO: 279 - DNA, 28 bp, Mus musculus
ccaagttttcattatggcttatttcaaa

SEQ ID NO: 280 - DNA, 28 bp, Mus musculus
tgtgctttgaaataagccataatgaaaa

SEQ ID NO: 281 - DNA, 28 bp, Mus musculus
gattttaaaaattgaaataatctccaag

SEQ ID NO: 282 - DNA, 28 bp, Mus musculus
gaaaacttggagattatttcaattttta

SEQ ID NO: 283 - DNA, 28 bp, Mus musculus
agagatataattattttaaaaattgaaa

SEQ ID NO: 284 - DNA, 28 bp, Mus musculus
gattatttcaatttttaaaataattata

SEQ ID NO: 285 - DNA, 29 bp, Mus musculus
ggattccatatactaaaaaaatagagata

SEQ ID NO: 286 - DNA, 29 bp, Mus musculus
gattatatctctatttttttagtatatgg

SEQ ID NO: 287 - DNA, 28 bp, Mus musculus
agctagttatagtttaggattccatata

SEQ ID NO: 288 - DNA, 28 bp, Mus musculus
tttagtatatggaatcctaaactataac

(19) United States
(12) Patent Application Publication
(10) Pub. No.: US 2010/0166784 A1
(43) Pub. Date: Jul. 1, 2010

(54) METHOD AND COMPOSITIONS FOR MODULATING TH17 CELL DEVELOPMENT

(75) Inventors: Kenneth M. Murphy, St. Louis, MO (US); Theresa Murphy, St. Louis, MO (US); Kai Hildner, St. Louis, MO (US); Barbara Schraml, St. Louis, MO (US)

Correspondence Address: POLSINELLI SHUGHART PC, 100 SOUTH FOURTH STREET, SUITE 100, SAINT LOUIS, MO, 63102-1825, US

(73) Assignee: THE WASHINGTON UNIVERSITY, St. Louis, MO (US)

(21) Appl. No.: 12/650,291

(22) Filed: Dec. 30, 2009

Related U.S. Application Data
(60) Provisional application No. 61/141,612, filed on Dec. 30, 2008.

(51) Int. Cl. A61K 39/00; C07H 21/04; C12N 15/63; C12N 5/10

(52) U.S. Cl. 424/184.1; 536/23.1; 536/24.1; 435/320.1; 435/325

(57) ABSTRACT

The invention encompasses methods and compositions for modulating Th17 development.

### CROSS REFERENCE TO RELATED APPLICATIONS

This application claims the priority of U.S. provisional application No. 61/141,612, filed Dec. 30, 2008, which is hereby incorporated by reference in its entirety.

### REFERENCE TO SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821 (f).

### FIELD OF THE INVENTION

The invention encompasses methods and compositions for modulating Th17 cell development.

### BACKGROUND OF THE INVENTION

T helper (Th) 17 and regulatory T (Treg) cells are recently described subsets of CD4⁺T cells that play critical opposing roles in a variety of inflammatory disorders. Pro-inflammatory Th17 cells are characterized by the production of a distinct profile of effector cytokines, including IL-17 (or IL-17A), IL-17F, and IL-6, whereas anti-inflammatory Treg cells play an important role in the preservation of self-tolerance and prevention of autoimmunity.

### SUMMARY OF THE INVENTION

One aspect of the present invention encompasses a method of modulating an immune response. The method comprises modulating Th17 cell development.

Another aspect of the present invention encompasses a method of modulating Th17 cell development. The method comprises modulating Batf expression.

Yet another aspect of the present invention encompasses an isolated nucleic acid comprising a Batf binding site.

Other aspects and iterations of the invention are described more thoroughly below.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

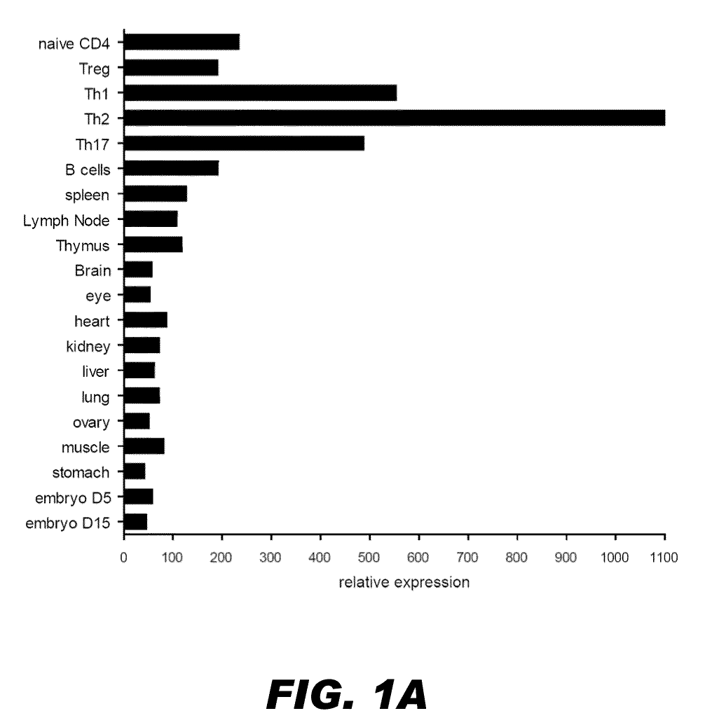

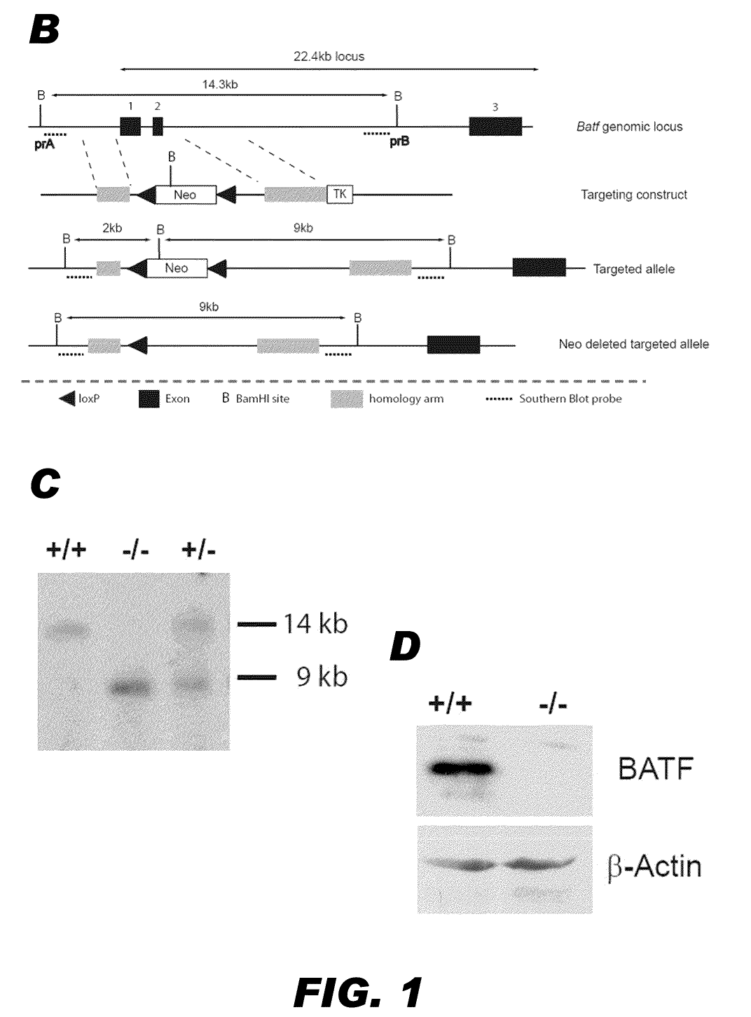

FIG. 1 depicts targeting of the Batf locus by homologous recombination. a, The expression profile of Batf among the indicated tissues was determined by Affymetrix gene microoarray. The data are presented in arbitrary units and reflect normalized and modeled expression values generated using DNA-Chip analyzer (dChip) software. b, The endogenous genomic Batf locus, targeting construct and the mutant allele before and after cre-mediated deletion of the neomycin cassette are shown. Restriction enzyme digestion with BamHI of the genomic locus results in a 14.3 kb wild type fragment that is detected by Southern Blot probes A and B; in the targeted allele, probe A detects a 2 kb and probe B detects a 9 kb fragment. In the neomycin-deleted targeted allele, BamHI digestion results in a 9 kb fragment that is detected by both the 5′ and 3′ Southern Blot probes. The neomycin resistance cassette was deleted by in vitro transfection with a Cre-expressing Adenovirus. c, Southern Blot analysis of targeted Batf alleles. Probe A was used to hybridize BamHI digested genomic DNA from the indicated genotypes resulting from Batf^{+/-} intercrosses. d, No residual protein expression Batf^{-/-} mice. Total splenocytes were activated under TH17 conditions for three days. Equal cell numbers were lysed in RIPA buffer and subjected to Western Blot analysis using anti-Batf antibody. The blots were stripped and reblotted with an antibody to β-actin to show equal protein loading.

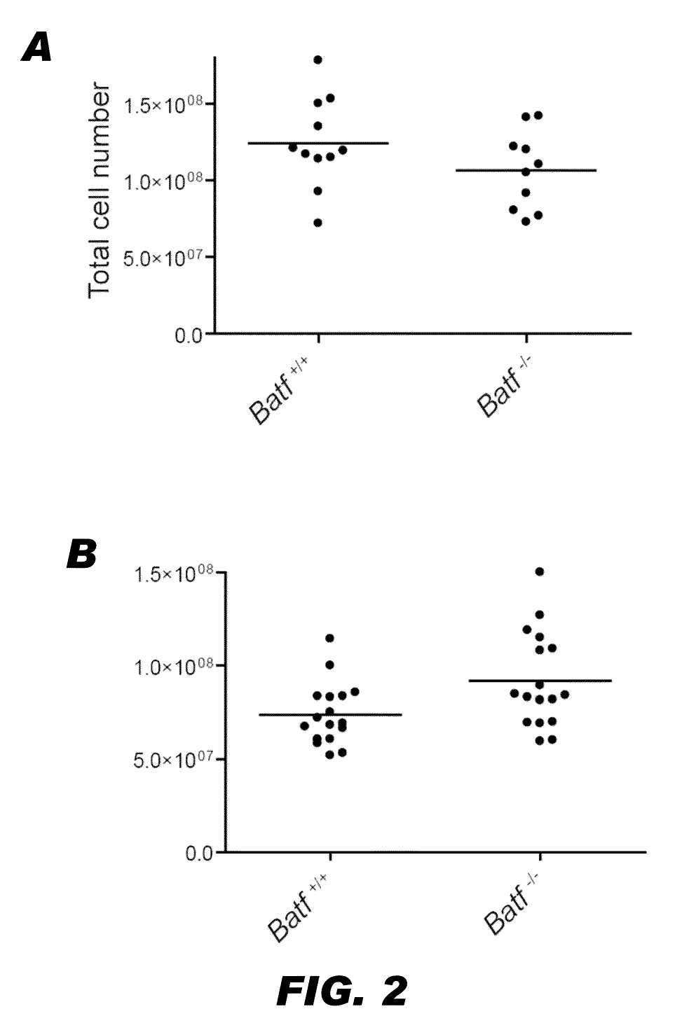

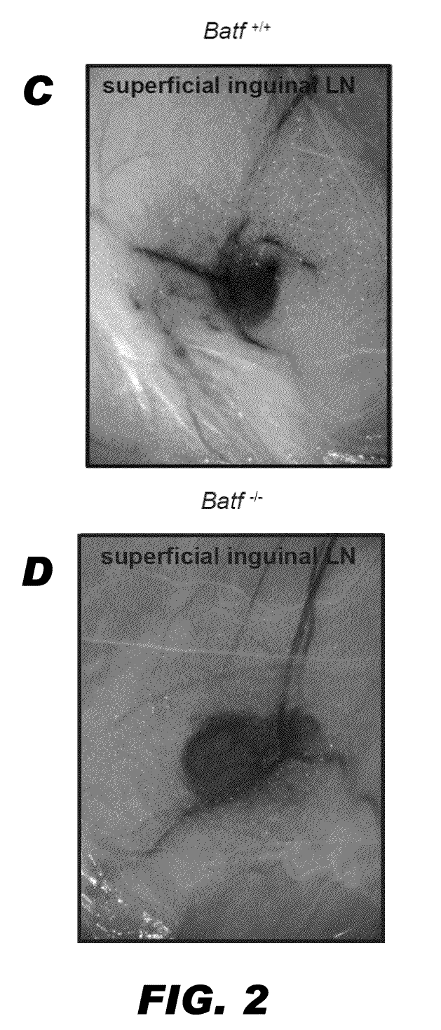

FIG. 2 shows that thymus, spleen and lymph nodes develop normally in Batf^{-/-} mice. a, Total cell numbers of thymus (n=11) and b, spleen (n=17) from individual 8-10 week old Batf^{+/+} and Batf^{-/-} mice are shown (horizontal bars indicate mean cell numbers). c, d, Batf^{+/+} and Batf^{-/-} mice were injected with Evans Blue dye solution into each hind foot pad. After 1.5 hrs, mice were sacrificed and superficial inguinal lymph nodes were visualized using a dissecting microscope.

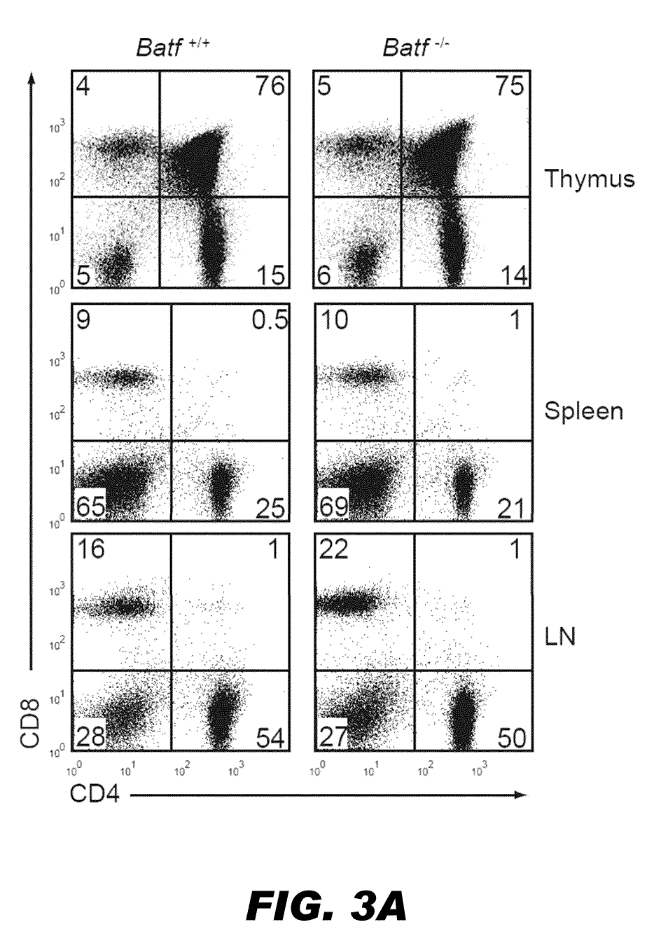

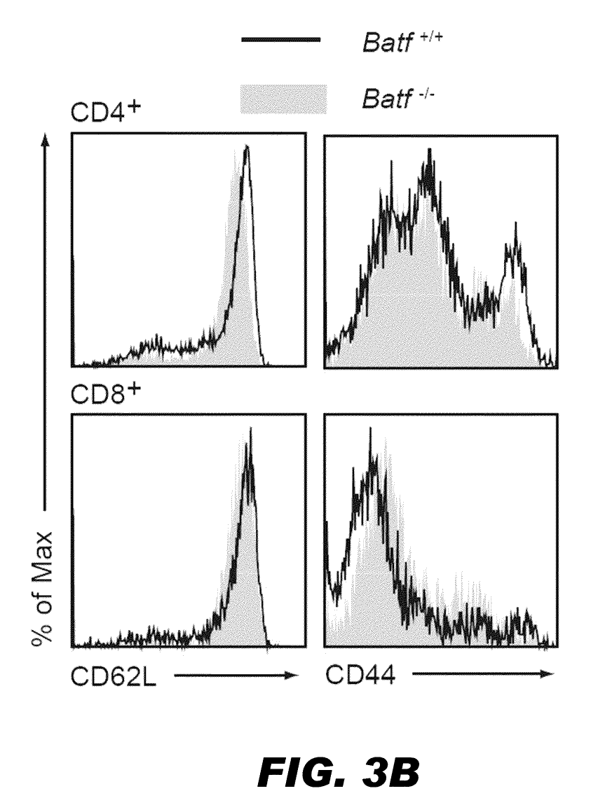

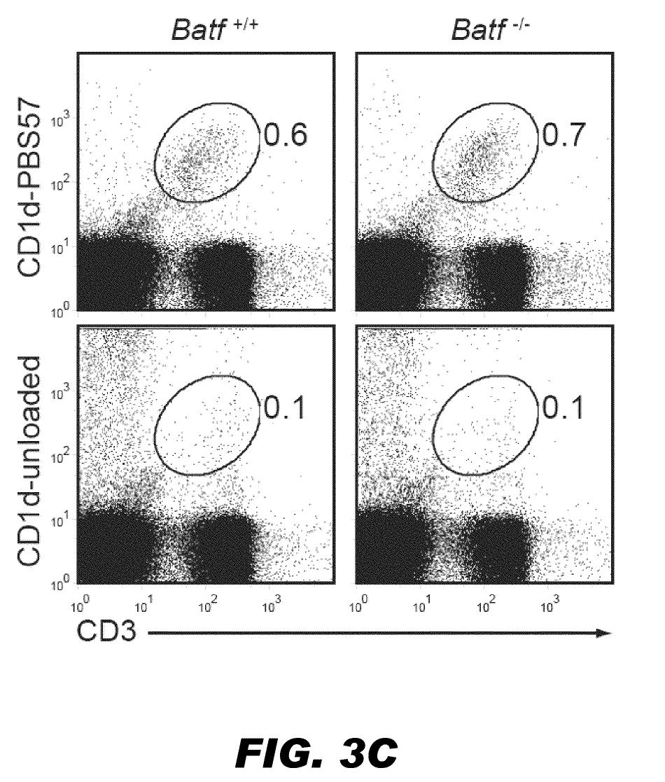

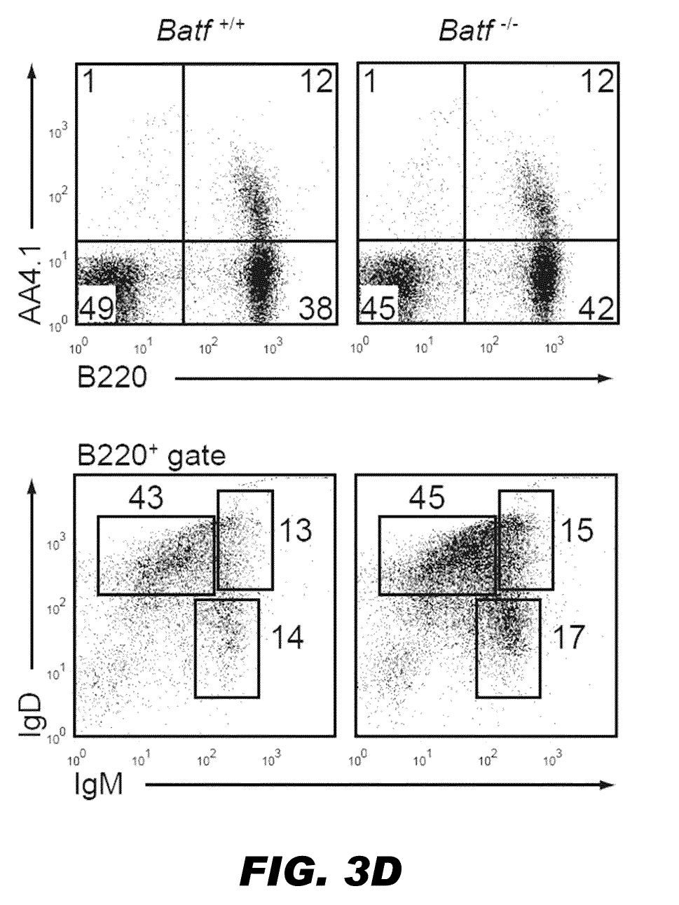

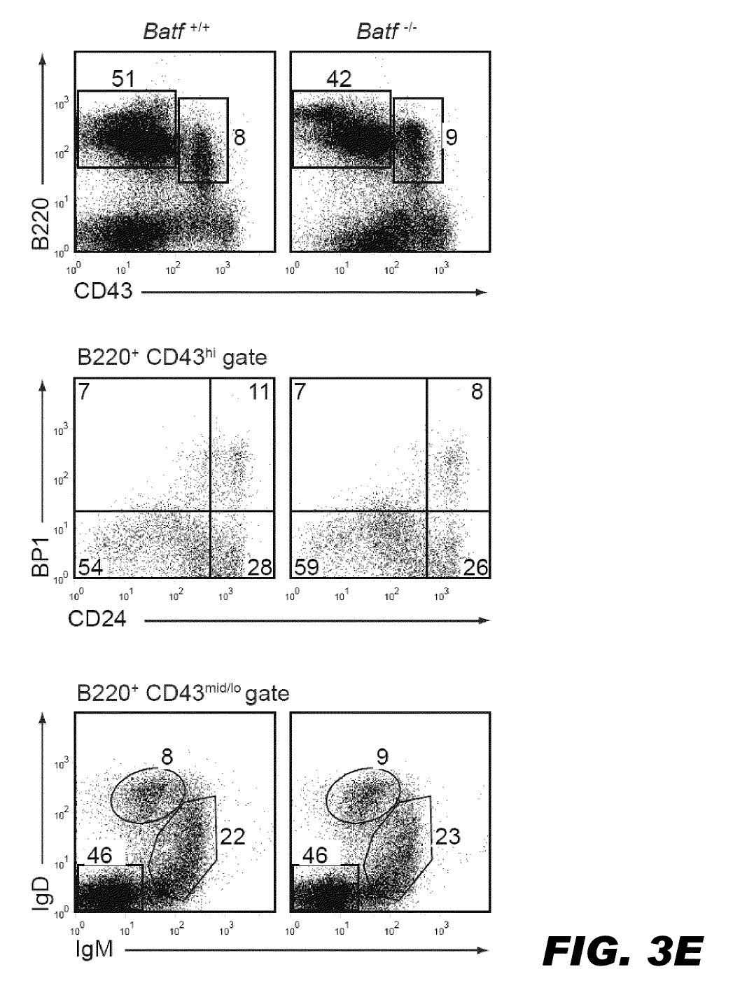

FIG. 3 depicts the normal development of T and B cells in Batf^{-/-} mice. a, Thymus, spleen and lymph nodes of mice of the indicated genotypes were analyzed for the surface expression of CD4 and CD8 by flow cytometry. The percentages of CD8⁺, CD4⁺ and CD4⁺CD8⁺T cells were similar between Batf^{+/+} and Batf^{-/-} mice. b, Splenic CD4⁺ and CD8⁺ cells were analyzed for the surface expression of the activation markers CD62L (left panel) and CD44 (right panel) on Batf^{+/+} and Batf^{-/-} cells. A histogram overlay of surface expression of CD62L and CD44 on Batf^{+/+} and Batf^{-/-}CD4⁺ and CD8⁺T cells is shown. c, Total splenocytes were stained for CD3 in conjunction with unloaded or PBS57-loaded CD1d tetramers. NKT cells are identified as CD3⁺CD1d-PBS57⁺. d, Total splenocytes were analyzed by staining with antibodies to B220, AA4.1, IgM and IgD. The percentages of immature B cells (AA4.1⁺B220⁺), Transitional 1 (B220⁺IgM^{hi}IgD^{lo}, Transitional 2 (B220⁺IgM^{hi}, IgD^{lo}) or mature B cells (AA4.1⁻B220⁺; B220⁺IgM^{lo}IgD^{hi}) were similar between Batf^{+/+} and Batf^{-/-} mice. e, Bone Marrow cells were stained for the expression of B220, CD43 and either BP1 and CD24 or IgD and IgM. The percentages of cells included in B220⁺CD43^{hi} subsets: BP-1⁻CD24⁻ (Hardy fraction A), BP-1⁻CD24⁺ (Hardy fraction B), and BP-1⁺CD24⁺ (Hardy fraction C) were similar between Batf^{+/+} and Batf^{-/-} mice. Also the percentages of B220⁺CD43⁻ subsets; IgM⁻IgD⁻ (Hardy fraction D), IgM⁺IgD^{lo} (Hardy fraction E), and IgM^{lo}IgD^{hi} (Hardy fraction F) were similar between Batf^{+/+} and Batf^{-/-} mice. Numbers indicate percentage of cells in the indicated region or gate.

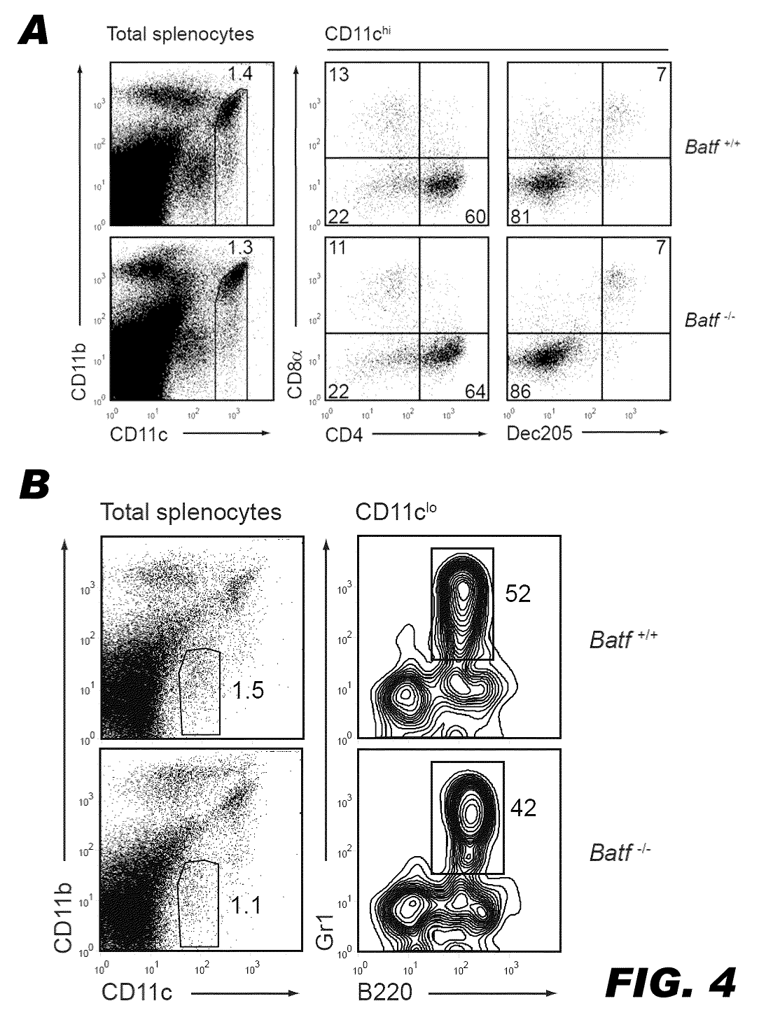

FIG. 4 depicts the development of myeloid cells is grossly normal in Batf^{-/-} mice. a, Conventional splenic dendritic cell (cDC) subsets are present at normal ratios in Batf^{-/-} mice. Single cell suspensions from collagenase and DNase treated spleens were stained with the indicated antibodies. cDCs were identified as CD11c^{hi} cells and further subdivided into CD4⁺DCs and CD8⁺DCs, identified as CD11c^{hi}CD4⁺CD8⁻ and CD11c^{hi}CD4⁻CD8α⁺ respectively. CD8⁺DCs were further identified as CD11c^{hi}CD8α⁺Dec205⁺. Numbers indicate the percentage of live cells in each gate or region. b, Splenic single cell suspensions were prepared as in a and stained with antibodies to CD11c, CD11b, Gr1 and B220. Percentages of plasmacytoid dendritic cells, identified as CD11b⁻CD11c^{lo}B220⁺Gr1⁺, were similar between Batf^{+/+} and Batf^{-/-} mice. Numbers indicate the percentage of live cells in each gate or region.

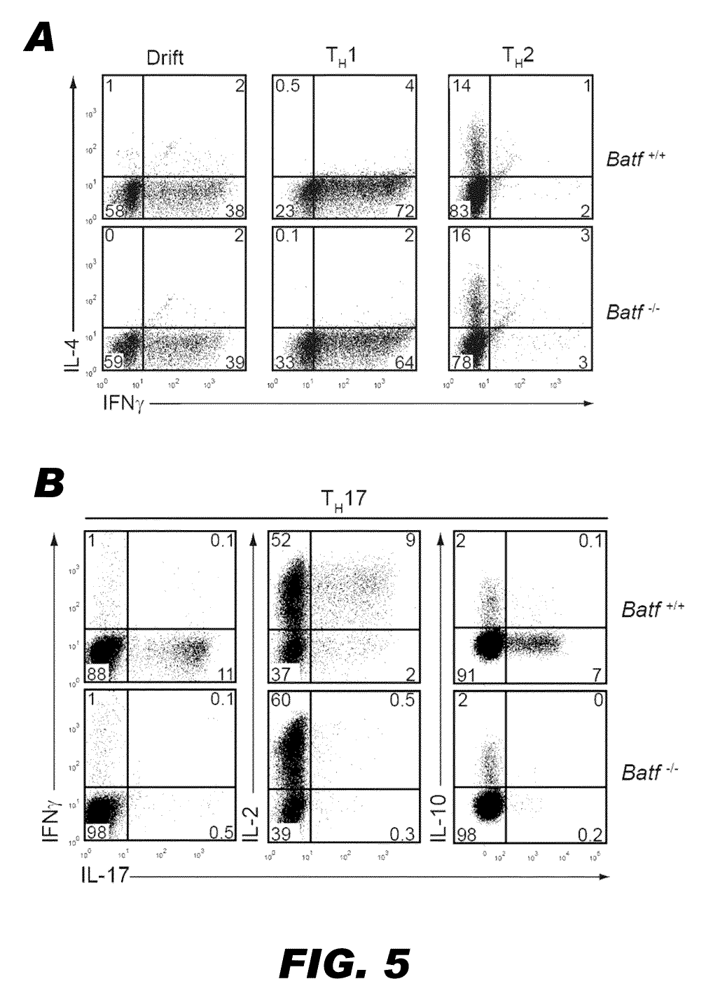

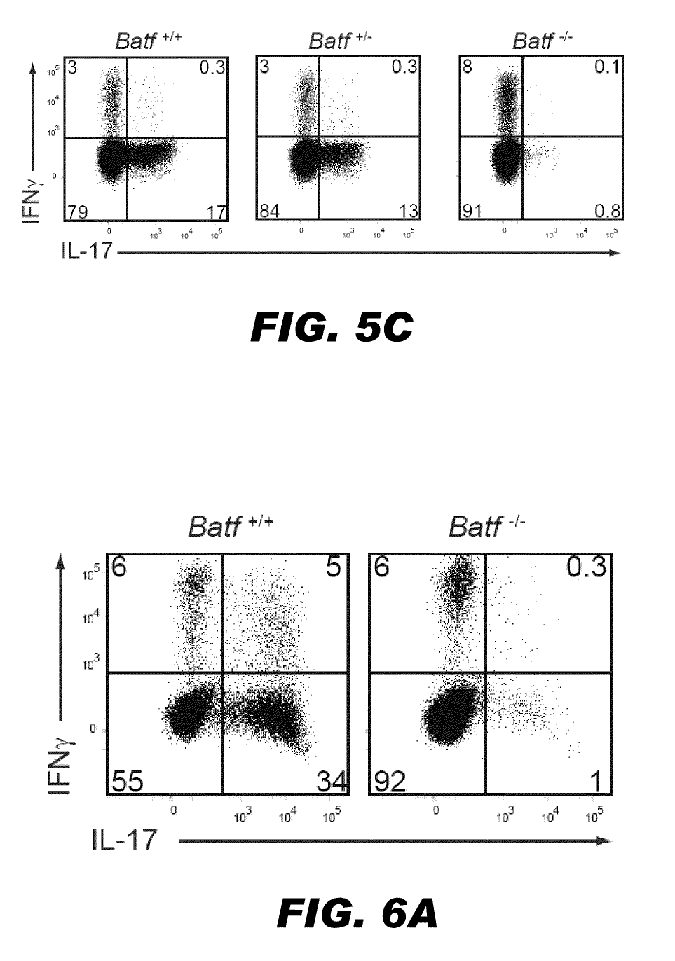

FIG. 5 depicts the selective loss of IL-17 production in Batf^{-/-}T cells. a, Naïve CD4⁺CD62L⁺CD25⁻T cells from Batf^{+/+} and Batf^{-/-} mice activated under drift, TH1 or TH2 conditions were analyzed for IFN-γ and IL-4 production 7 days after stimulation. b, Naïve CD4+CD62L+CD25⁻T cells from Batf^{+/+} and Batf^{-/-} mice were activated under TH17 conditions as described in Methods, restimulated on day 7 (left panel) or day 3 (middle and right panels) and stained for intracellular IL-17, IFN-γ, IL-2 and IL-10. c, D011.10 transgenic CD4⁺T cells from Batf^{+/+}, Batf^{+/-} and Batf^{-/-} mice were stimulated with OVA and APC under Th17 conditions, and stained for intracellular IL-17 and IFN-γ. Numbers represent the percentage of live cells in the indicated gate. Data are representative of at least 2 independent experiments performed with multiple mice of each genotype.

FIG. 6 depicts data showing that Batf regulates IL-17 production by CD4⁺ and CD8⁺ cells. a, CD4⁺T cells from D011.10 Batf^{+/+} and Batf^{-/-} mice were purified by magnetic bead separation and activated with OVA and irradiated APCs under TH17 conditions. Three days later, cells were split and allowed to expand for four days in the presence of TH17 inducing cytokines. After 3 rounds of differentiation, cells were restimulated with PMA/ionomycin for 4 hours and analyzed for IFN-γ and IL-17 expression by flow cytometry. Numbers indicated the percentage of live cells in each gate or region. b, Total splenocytes from Batf^{+/+} and Batf^{-/-} were stimulated under TH17 conditions for three days. Cells were restimulated with PMA/ionomycin and analyzed for IL-17 and IFNγ expression by intracellular cytokine staining and flow cytometry. Plots are gated on CD8⁺ cells and numbers indicate the percentage of live cells in each gate or region. c, D011.10 transgenic CD4⁺T cells from CD2-Batf transgenic (TG) or transgenenegative (WT) control mice were stimulated with OVA and APC under TH17 conditions. Three days later, cells were restimulated with PMA/ionomycin and cytokine production was analyzed by flow cytometry as described in methods. d, Total splenocytes from CD2-Batf transgenic (TG) or transgene-negative (WT) control mice were stimulated and analyzed as in b. e, Small intestinal lamina propria cells were isolated from Batf^{+/+} and Batf^{-/-} mice and stimulated with PMA/ionomycin as described in Methods and stained for IL-17 and IFN-γ production. Plots are gated on CD4⁺ lymphocytes. Numbers indicate the percentage of live cells in each indicated gate. Data are representative of at least 2 independent experiments performed with multiple mice of each genotype.

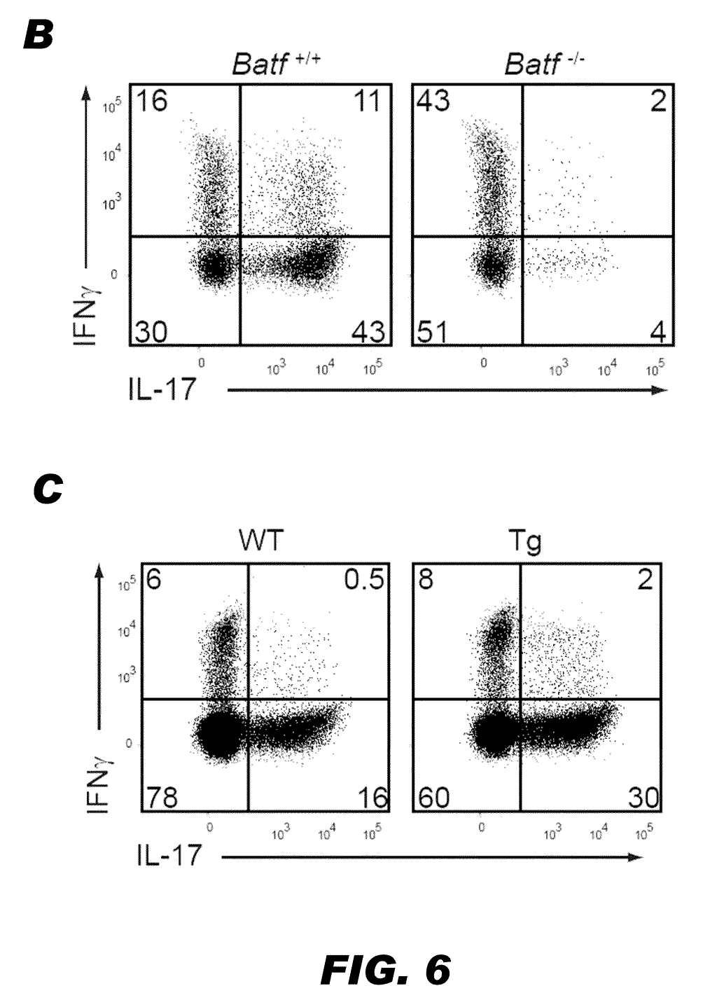

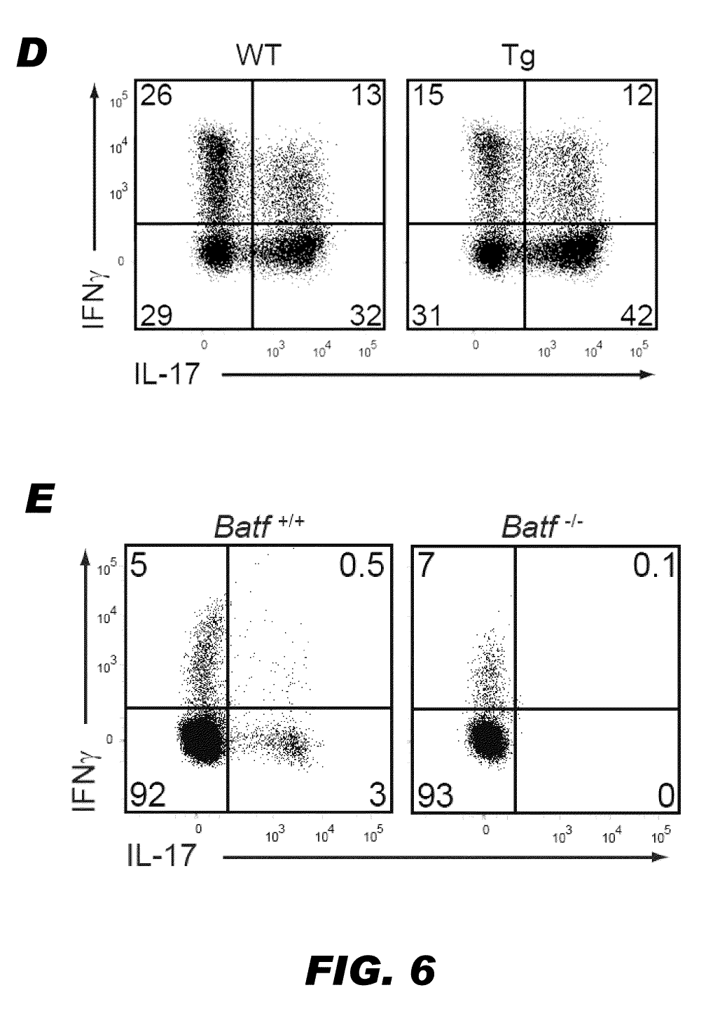

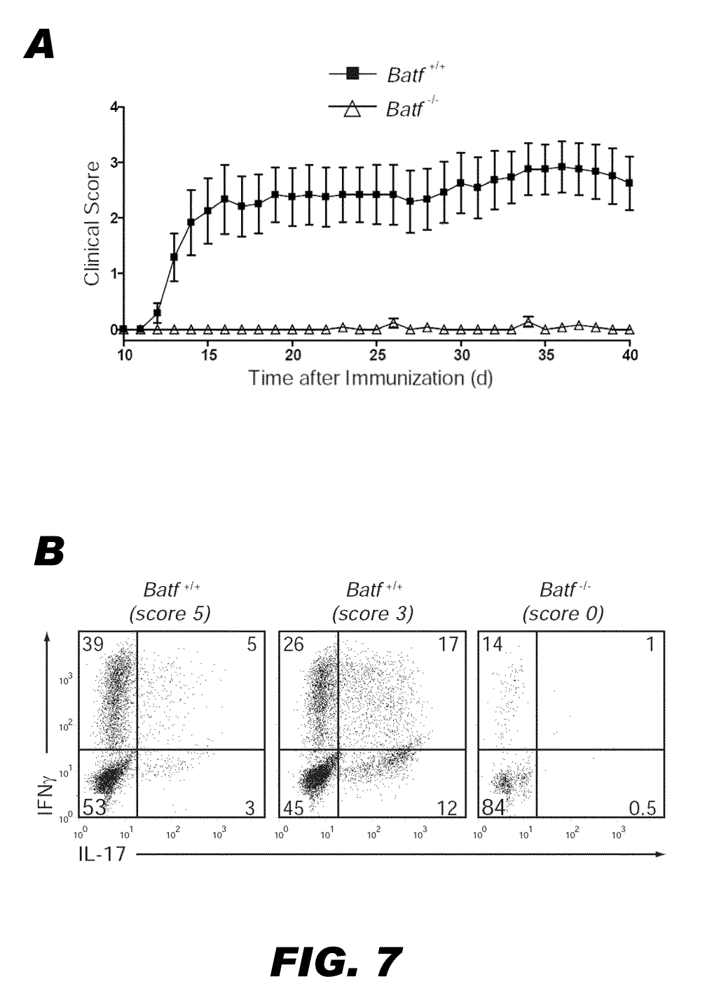

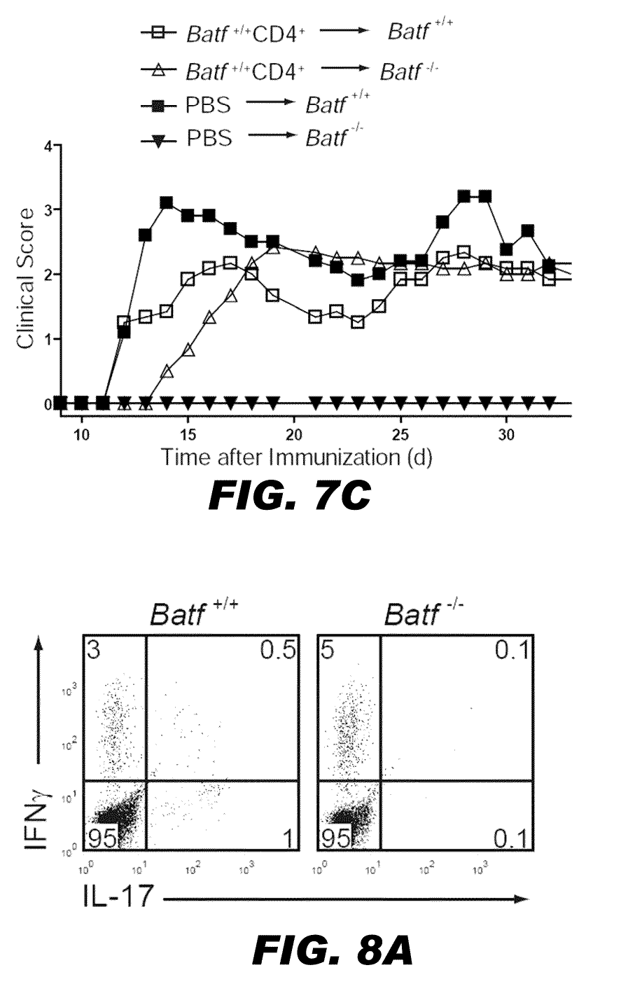

FIG. 7 depicts the resistance of Batf^{-/-} mice to EAE. a, Batf^{+/+} (n=12) and Batf^{-/-} (n=13) mice were immunized with MOG33-35 peptide as described in Methods. Clinical EAE scores (mean+/−s.e.m) representative of two independent experiments are shown. b, 13 days after EAE induction, CNS infiltrating lymphocytes were stimulated with PMA/ionomycin for 4 hrs and stained for intracellular IL-17 and IFN-γ. Plots are gated on CD4⁺ lymphocytes. Clinical scores are shown in parentheses. Data are representative of 2-3 mice analyzed per group. c, Batf^{+/+} and Batf^{-/-} were injected with either control PBS buffer (n=5) or 1×10⁷ Batf^{+/+}CD4+T cells (n=6). Four days later, mice were immunized with MOG35-55 as in a. Mean clinical EAE scores are shown.

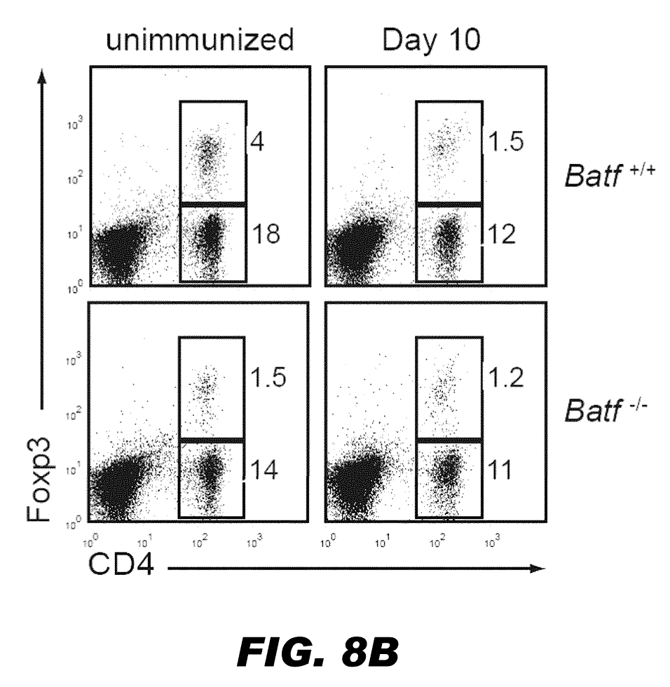

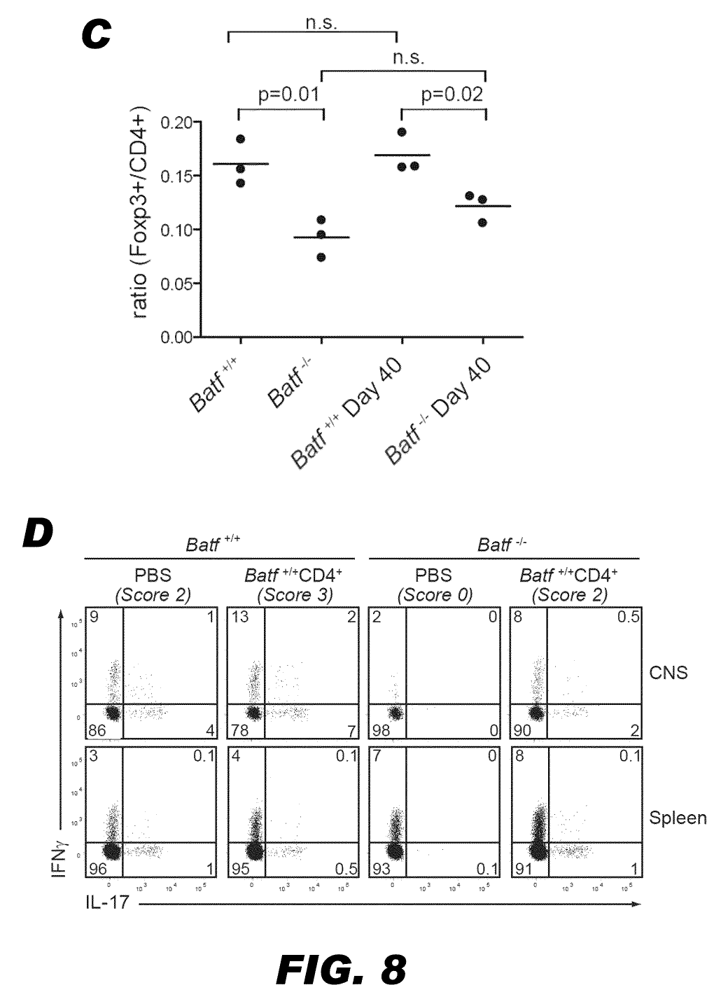

FIG. 8 depicts Batf^{-/-} mice are resistant to EAE. a, Total splenocytes were isolated from Batf^{+/+} and Batf^{-/-} mice 10 days after EAE induction, stimulated with PMA/ionomycin for 3 hours and analyzed for IL-17 and IFNγ expression by intracellular cytokine staining. Plots are gated on CD4⁺ cells. b, Spleens were isolated from unimmunized Batf^{+/+} and Batf^{-/-} or mice 10 days after EAE induction. Total splenocytes were stained for the expression of CD4 and Foxp3 and analyzed by flow cytometry. Numbers indicate percentage of cells in each indicate gate. c, Spleens were isolated from unimmunized Batf^{+/+} and Batf^{-/-} mice or mice 40 days after EAE induction. The abundance of Foxp3⁺ cells is depicted as the ratio of CD4⁺Foxp3⁺ cells in the total CD4⁺T cell compartment. d, Four days prior to EAE induction, Batf^{+/+} and Batf^{-/-} mice received either control buffer (PBS) or 1×10⁷ Batf^{+/+}CD4⁺T cells. 40 days after EAE induction splenic and CNS infiltrating lymphocytes were analyzed for IL-17 and IFN-γ production. Genotypes and whether mice received PBS or CD4⁺T cells are indicated, disease scores are given in parentheses. FACS plots are gated on CD4⁺ cells and are representative of 2-3 mice analyzed per group. Numbers indicate percentage of cells in each indicate gate.

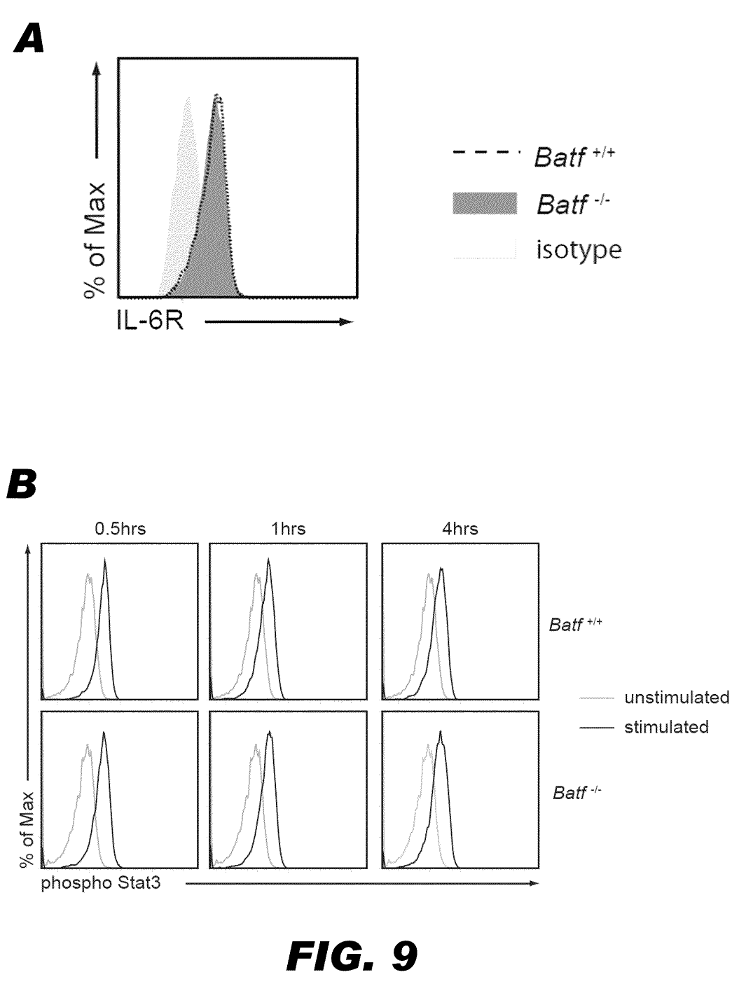

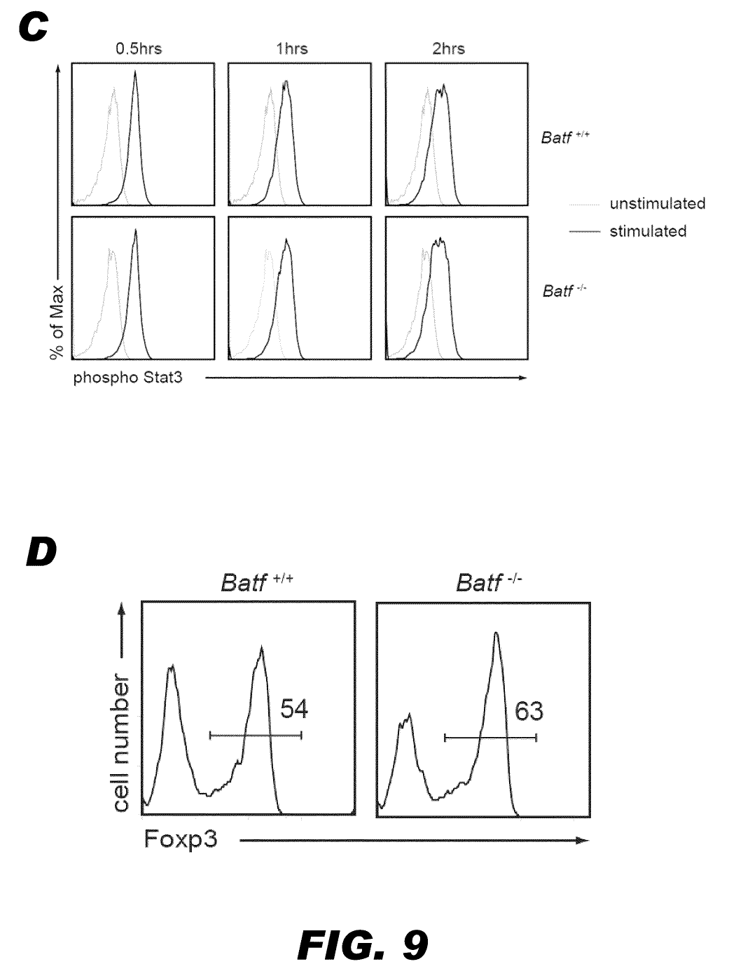

FIG. 9 depicts proximal IL-6 receptor signaling is normal in Batf^{-/-}T cells. a, Splenocytes from Batf^{+/+} and Batf^{-/-} mice were stained with antibodies to CD4 and IL-6 receptor (IL-6R). A histogram overlay of IL-6R expression on CD4⁺ cells of the indicated genotypes is shown. b, Magnetically purified Batf^{+/+} and Batf^{-/-} CD4+T cells were stimulated in the presence of IL-6 for the indicated times and stained with an antibody to phospho-STAT3 (black lines) by intracellular staining as described in methods. Unstimulated cells (grey lines) served as a negative control. c, Magnetically purified Batf^{+/+} and Batf^{-/-} CD4⁺T cells were stimulated in the presence of IL-21 for the indicated times and stained with an antibody to phospho-STAT3 (black lines) by intracellular staining. Unstimulated cells (grey lines) served as a negative control. d, Naïve CD4⁺CD62L⁺CD25⁻T cells from Batf^{+/+} and Batf^{-/-} mice were stimulated with TGF-β for three days. Cells were stained for Foxp3 and analyzed by flow cytometry.

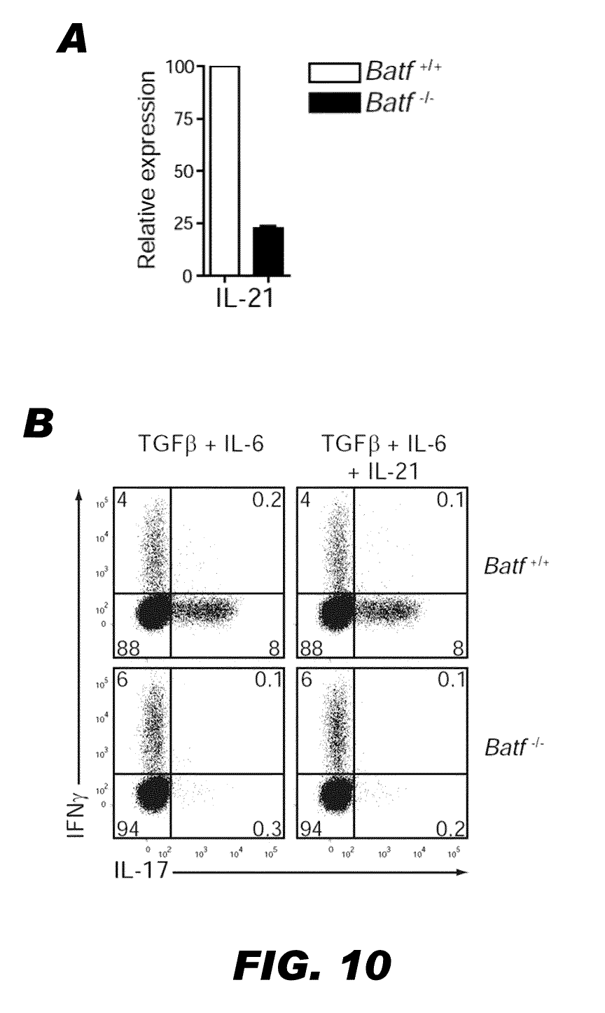

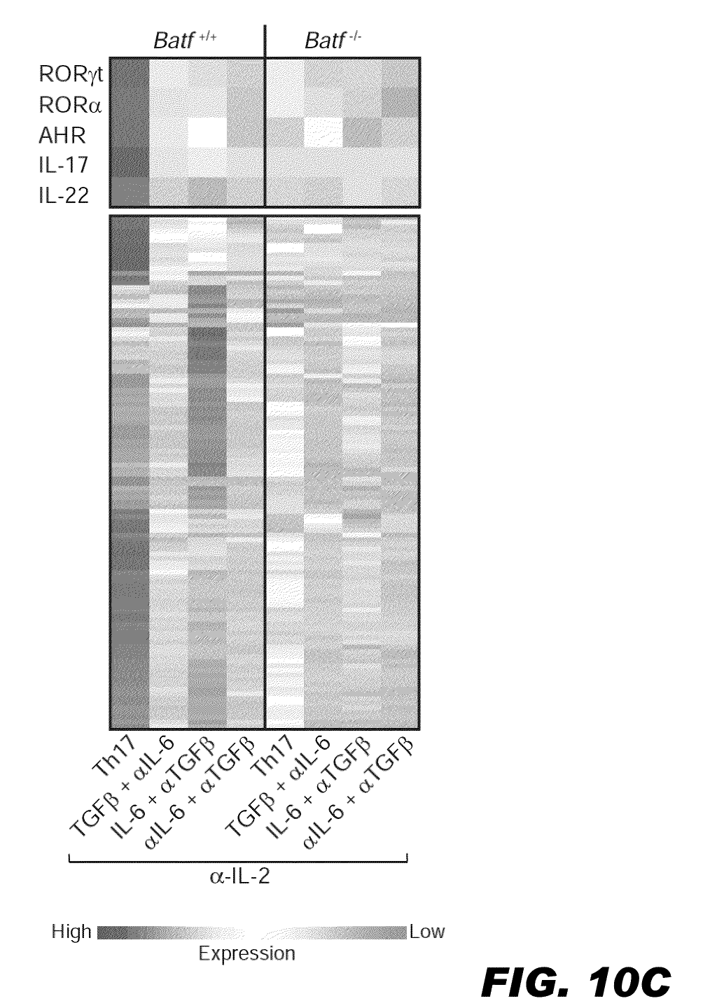

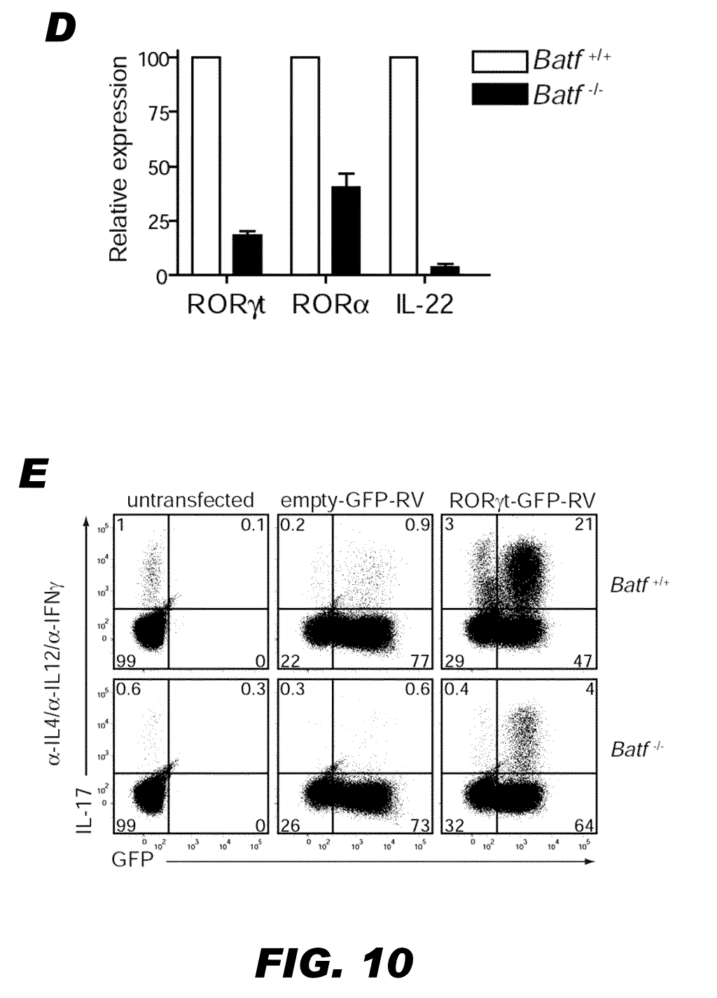

FIG. 10 depicts that Batf controls the expression of multiple TH17 associated genes. a, Relative expression of IL-21 in T cells 3 days after activation under TH17 conditions, assessed by qRT-PCR. Data in a and d are normalized to HPRT and presented as percent expression relative to Batf^{+/+} cells (mean±s.d. of 3 individual mice). b, Naive CD4⁺CD62L⁺CD25⁻T cells were activated as in a in the presence or absence of IL-21 and stained for IL-17 and IFN-γ. c, Gene expression microarray analysis of T cells activated for 72 h in the presence of the indicated cytokines and antibodies. Representative heat maps of genes differentially expressed Batf^{+/+} and Batf^{-/-}T cells are presented. d, Relative expression of RORγt, RORγt and IL-22 in T cells 72 h after activation under TH17 conditions, assessed by qRT-PCR. e, CD4⁺T cells were activated as indicated, left untreated or infected with RORγt-GFP-RV or control-GFP-RV as described in Methods. GFP and IL-17 expression 3 days after activation is shown.

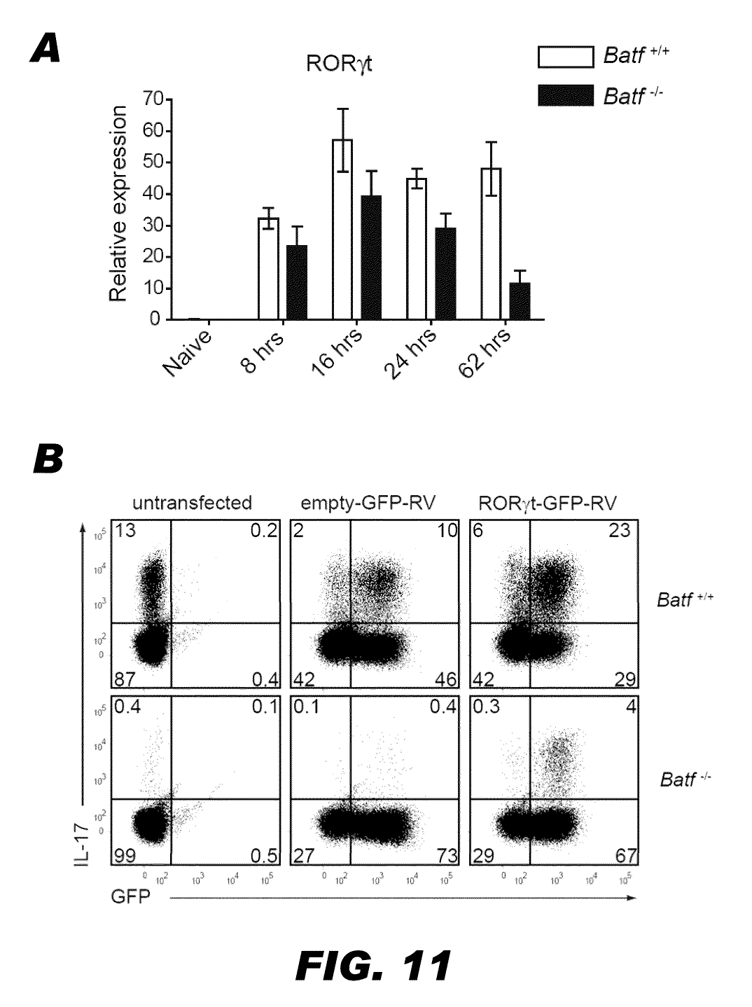

FIG. 11 depicts retroviral overexpression of RORγt fails to restore IL-17 production in Batf^{-/-}T cells. a, Naïve CD4⁺CD62L⁺CD25⁻T cells were stimulated under TH17 conditions for 0, 8, 16, 24 and 62 hours. Relative expression (normalized to HPRT) of RORγt in Batf^{+/+} and Batf^{-/-}T cells is depicted (error bars: mean±s.d. of 3 individual mice). b, Magnetically purified CD4⁺T cells were stimulated under TH17 conditions and either left untreated or infected with empty-IRES-GFP-retrovirus (GFP-RV) or RORγt expressing IRES-GFP-retrovirus (RORγt-RV) as described in Methods. Cells were restimulated with PMA/ionomycin and analyzed for cytokine expression on day 3. c, CD4⁺T cells were stimulated as indicated and infected with retrovirus as in (b) and FIG. 10e. The percentage of IL-17 producing cells among stably infected (GFP⁺) cells is shown (mean±s.d. of three independent experiments).

FIG. 12 depicts data showing that DLGH2 is an IL-6 induced Batf dependent gene.

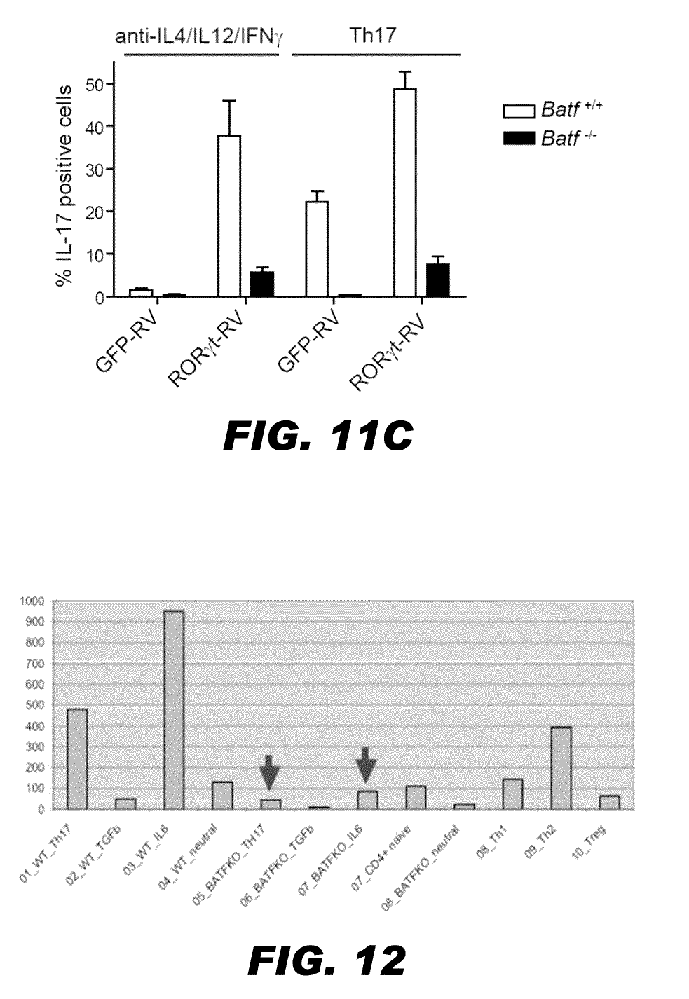

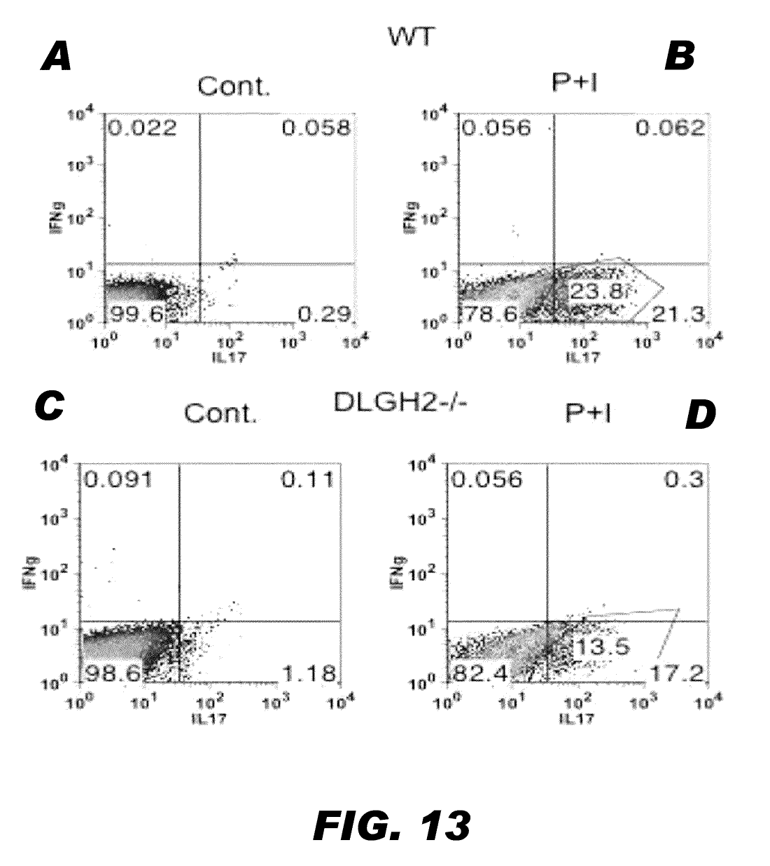

FIG. 13 depicts data showing the impaired Th17 differentiation in DLGH2^{-/-}T cells (c, d) compared to wt cells (a, b).

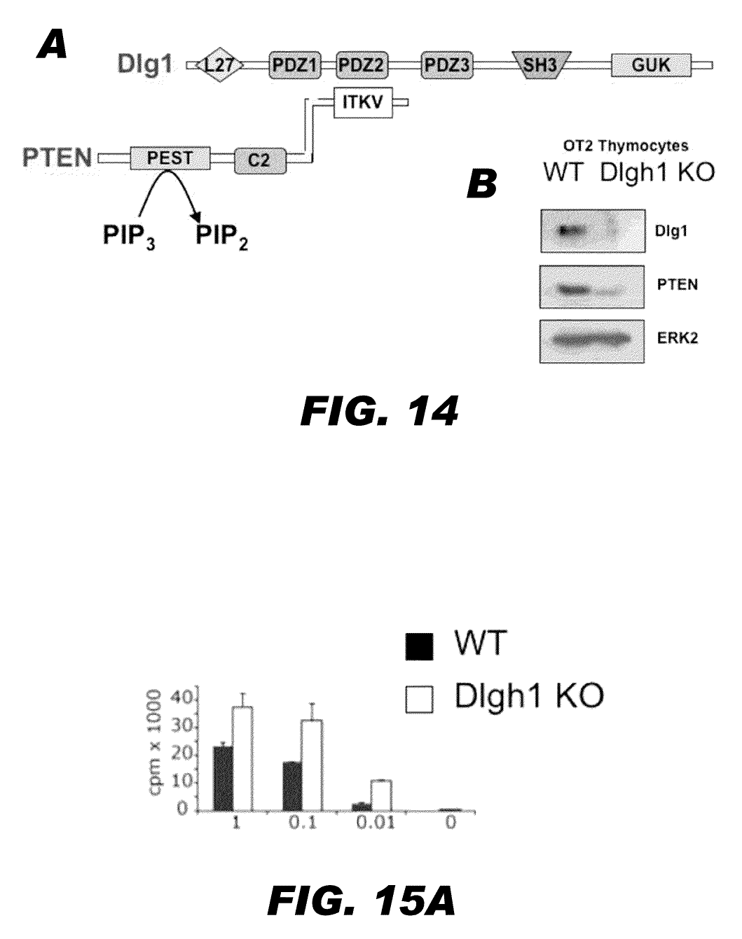

FIG. 14 depicts PTEN interaction with DLG (a), and data showing that Dlg stabilizes PTEN in lymphocytes (b). Dlg1 PDZ2 domain binds PTEN C-terminus, post-translationally enhancing PTEN stability.

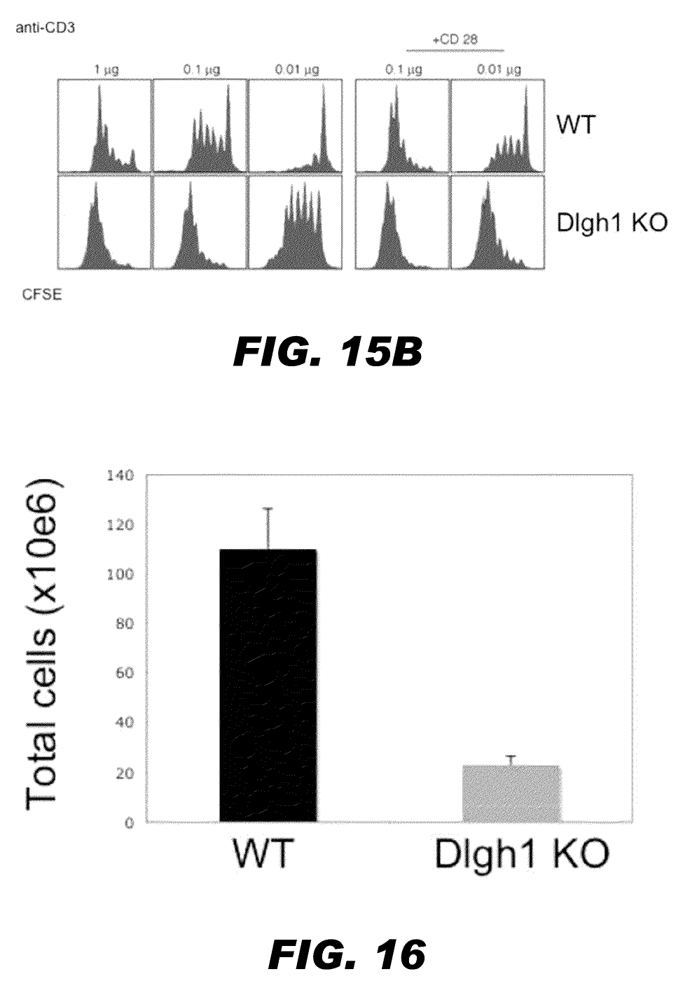

FIGS. 15 (a) and (b) depicts data showing that Dlg1 attenuates TCR signals-knockout approach.

FIG. 16 depicts data showing that Dlgh1 is required for thymocyte development.

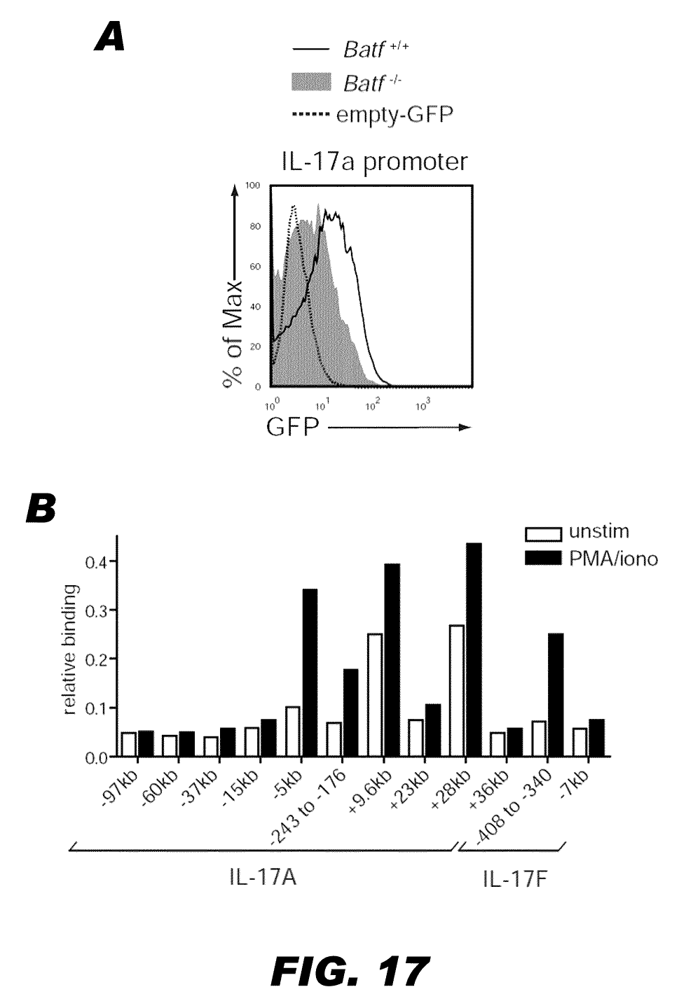

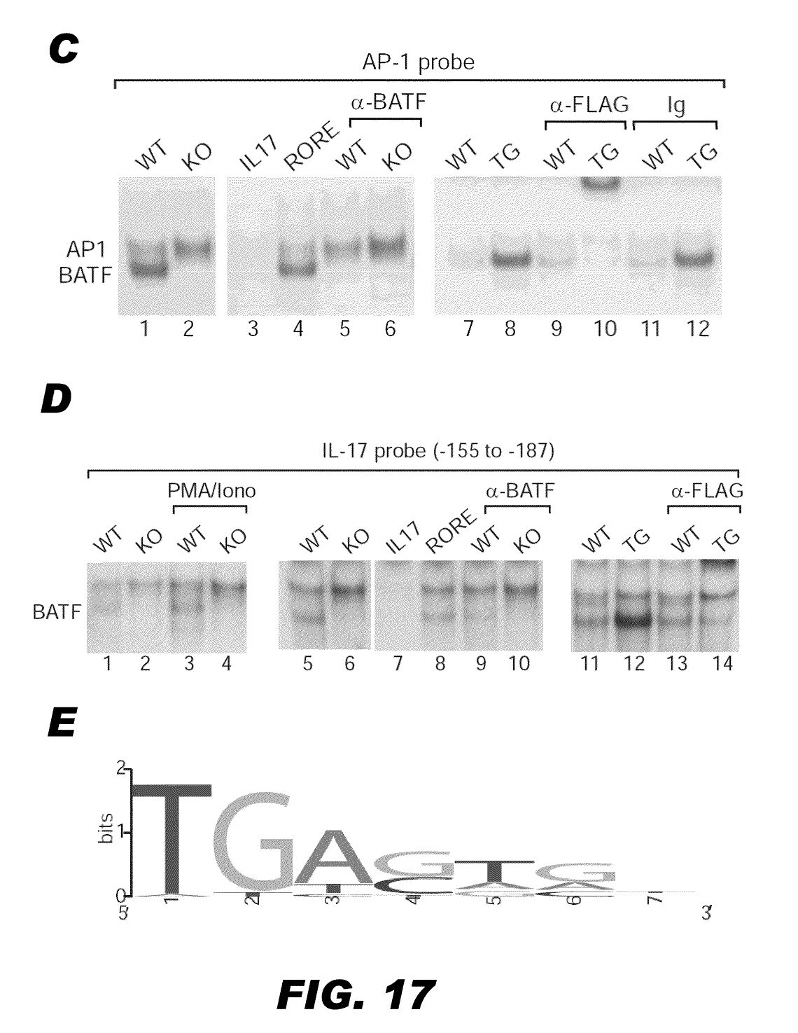

FIG. 17 depicts data showing that Batf directly regulates IL-17 expression. a, Batf^{+/+} and Batf^{-/-}CD4⁺T cells stimulated under TH17 conditions were infected with hCD4-pA-GFP-RV-IL-17p reporter virus. GFP expression in hCD4⁺ cells after restimulation with PMA/ionomycin is shown. hCD4-pA-GFP-RV infected cells served as negative control (dotted line). b, Batf^{+/+}CD4⁺T cells were stimulated under TH17 conditions for 5 days. ChIP analysis of T cells before and after PMA/ionomycin stimulation was performed using anti-Batf antibody. The analyzed sites are denoted relative to the ATG for the II17a or II17f genes. c-d, Whole cell extract from total splenocytes activated for 3 days under TH17 conditions were analyzed for binding to a consensus AP-1 probe (c) or the IL-17 (−155 to −187) probe (d). (Batf^{+/+} (WT), Batf^{-/-} (KO), CD2-Batf transgenic (TG)). e, WebLogo32 presentation of the 7-base Batf-binding motif identified by the CONSENSUS program31 present in 38/40 BATF-binding regions of the IL-17, IL-21 and IL-22 promoters. The size of each indicated nucleotide is proportional to the frequency of its appearance at each position.

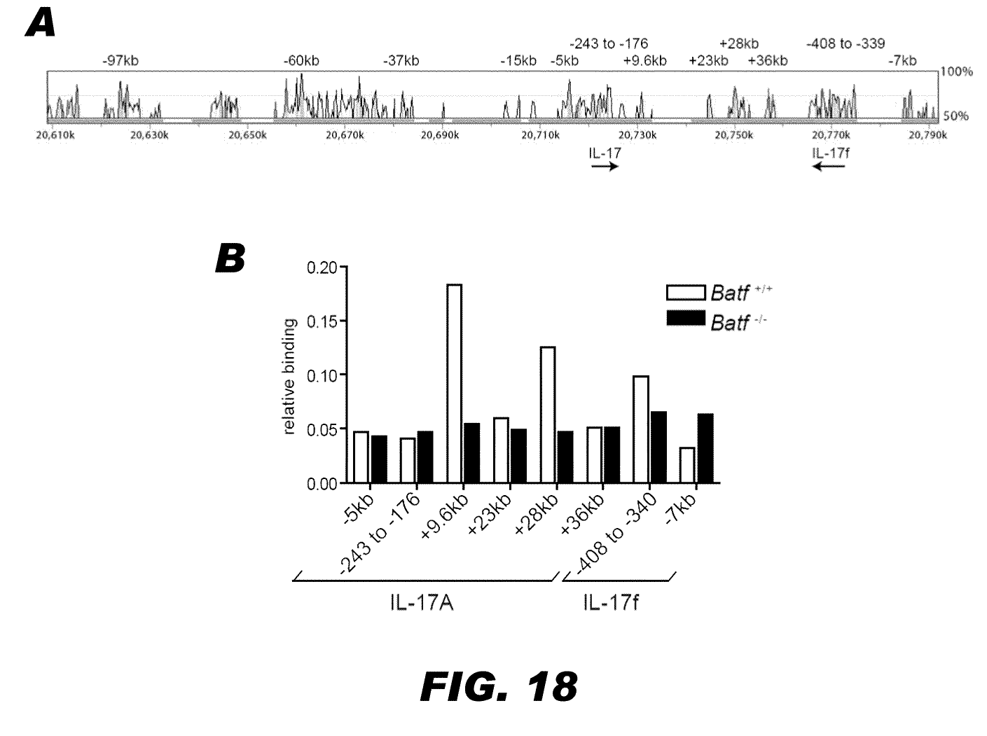

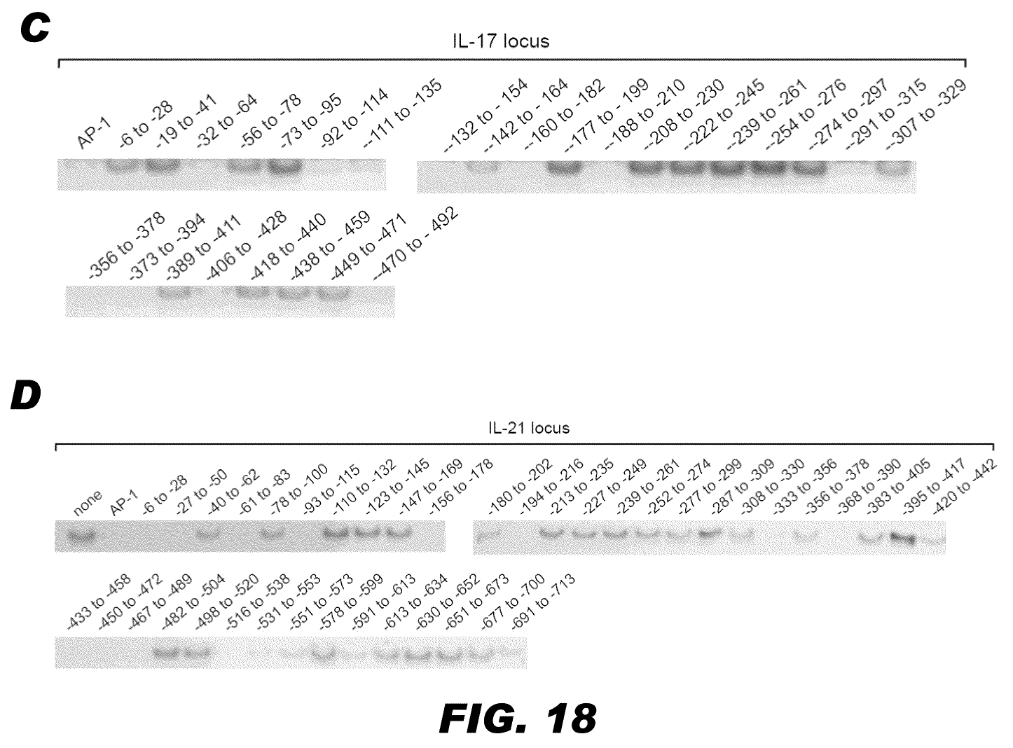

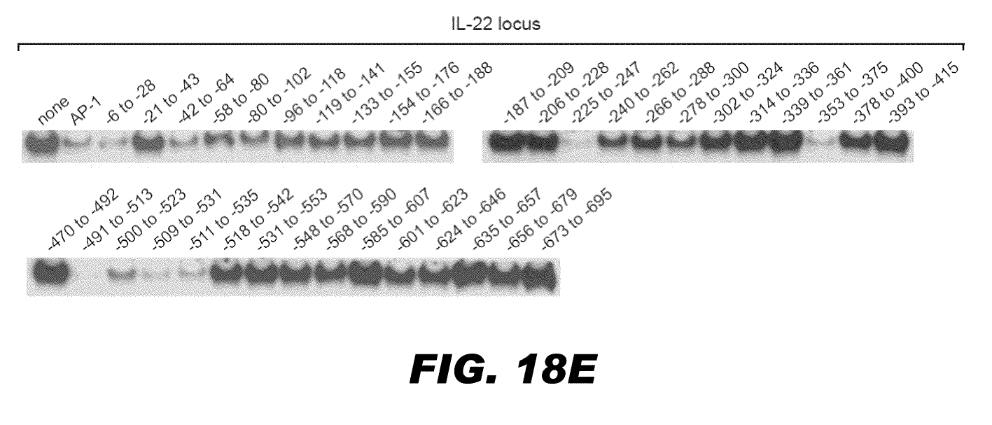

FIG. 18 depicts the identification of potential Batf binding sites in the IL-17a, IL-21 and IL-22 promoters. a, Vista blot depicting the sequence conservation of the human and mouse IL-17 loci. The locations of primers used for ChIP analysis are indicated. b, Specificity of ChIP analysis using anti-Batf antibody. Magnetically purified CD4⁺T cells from Batf^{+/+} or Batf^{-/-} mice were activated with anti-CD3/CD28 coated beads under TH17 conditions (IL-6/TGF-β) for 24 h, then processed for ChIP analysis using anti-Batf polyclonal antibody as in FIG. 17b. Data are expressed as relative binding based on normalization to unprecipitated input DNA. c-e, Identification of potential Batf binding sites in the IL-17, IL-21 and IL-22 promoters. Total splenocytes from Batf-transgenic mice were stimulated under TH17 conditions for three days. Total cell extracts were analyzed for DNA binding ability to a consensus AP-1 site by electrophoretic mobility shift assay. Batf containing complexes were identified by supershift with anti-FLAG antibody. Sequences from the IL-17a (c), IL-21 (d) and IL-22 (e) promoters were used to assess their ability to inhibit formation of Batf containing complexes as described in Methods.

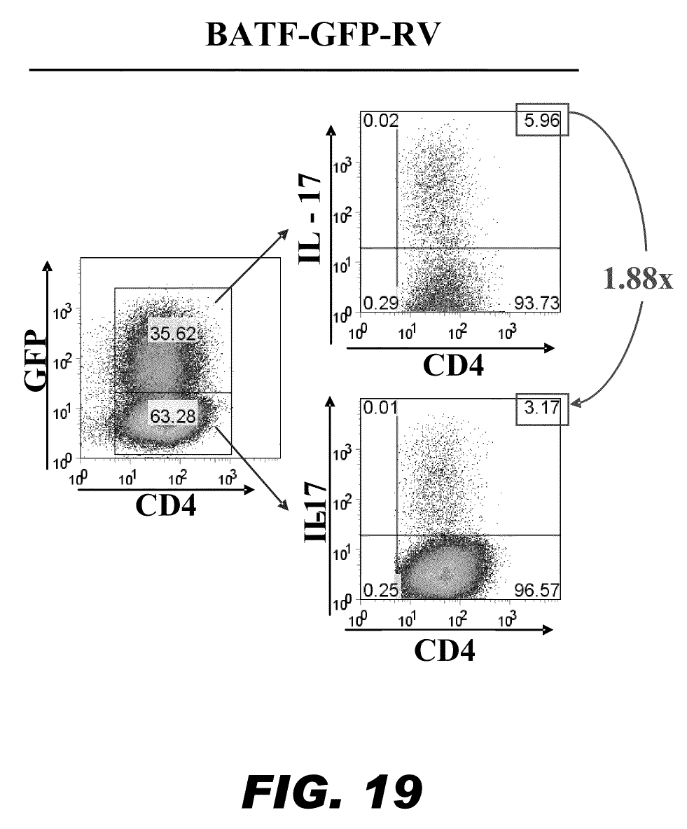

FIG. 19 depicts facs analysis showing Batf increases IL-17 production in human Th17 cells. HCB cells were retrovirally transduced with BATF during Th17 differentiation. IL-17 production by control (GFP-) and BATF expressing cells (GFP⁺) was determined by intracellular staining.

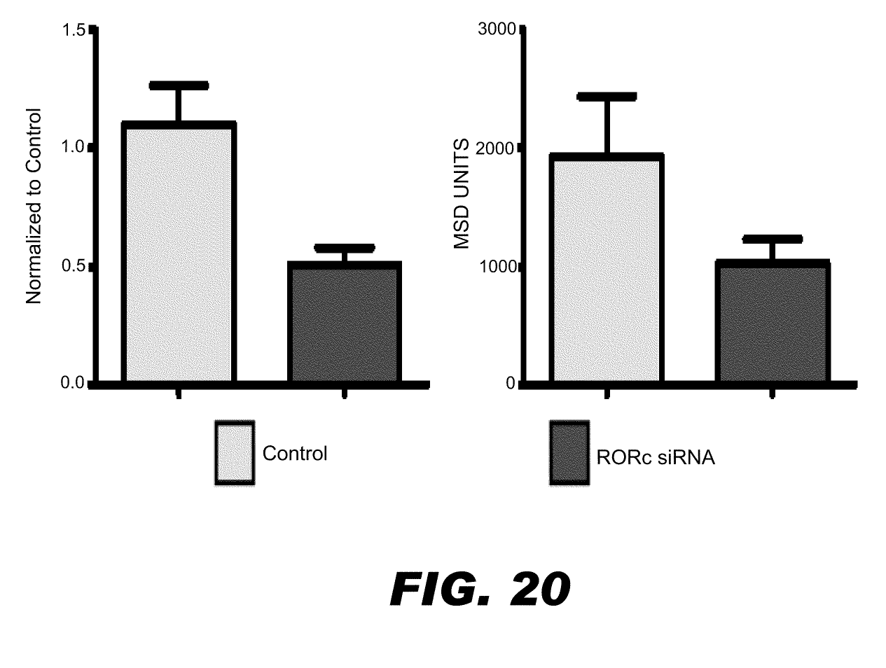

FIG. 20 depicts plots showing levels of IL-17 secretion from HCB derived Th17 cells. siRNA inhibition of RORγT reduces IL-17 secretion from HCB derived Th17 cells.

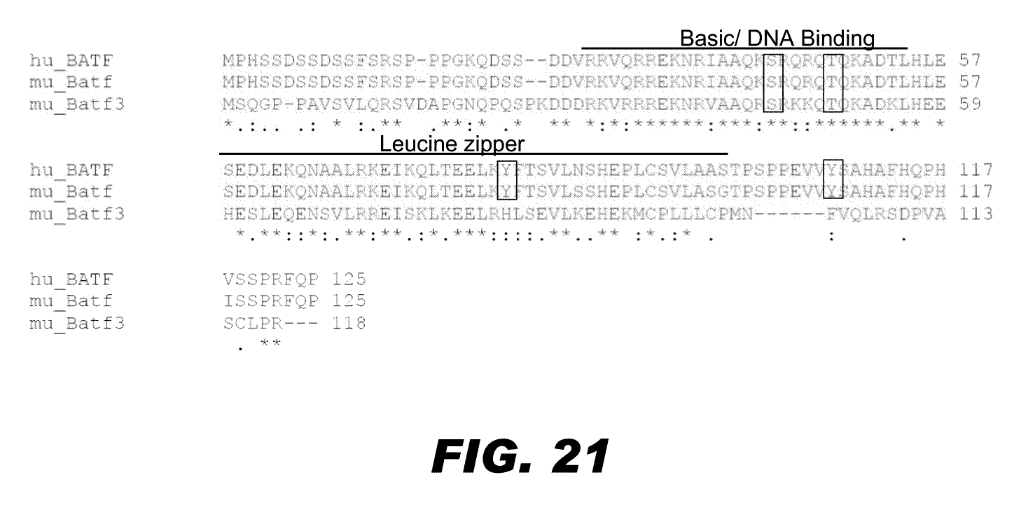

FIG. 21 depicts the amino acid sequence of mouse Batf (SEQ ID NO: 2) compared to human Batf (SEQ ID NO:289) and mouse Batf3 (SEQ ID NO:1).

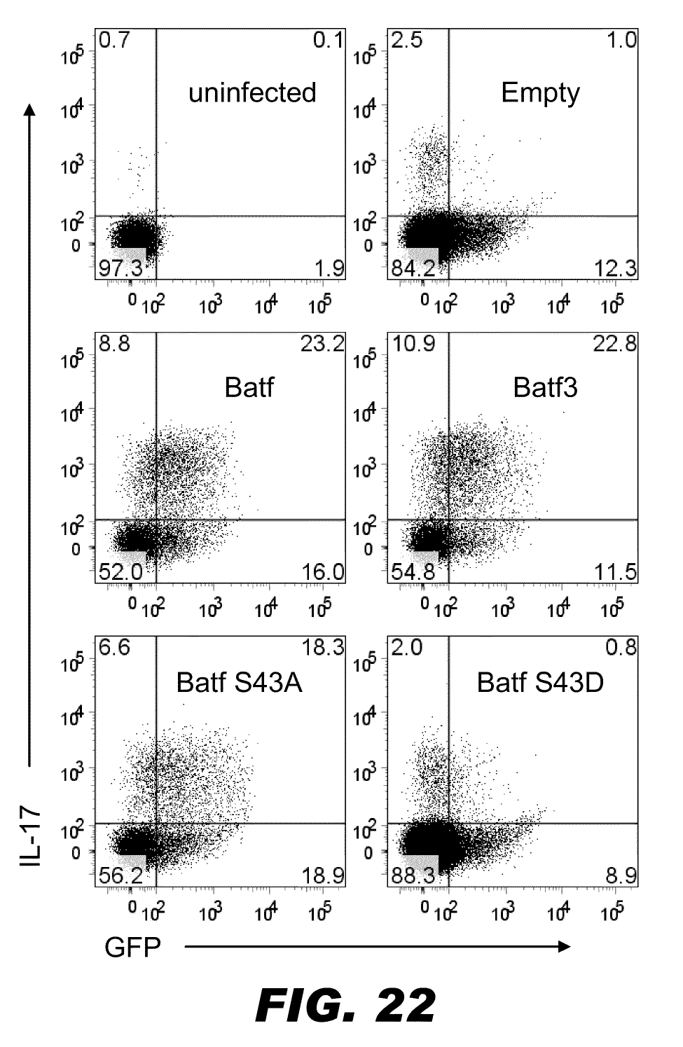

FIG. 22 depicts FACS analysis of Batf^{-/-} Batf3^{-/-}T cells left uninfected or retrovirally infected with the indicated cDNA. IL-17 production was measured in uninfected (GFP-) and infected (GFP+) cells.

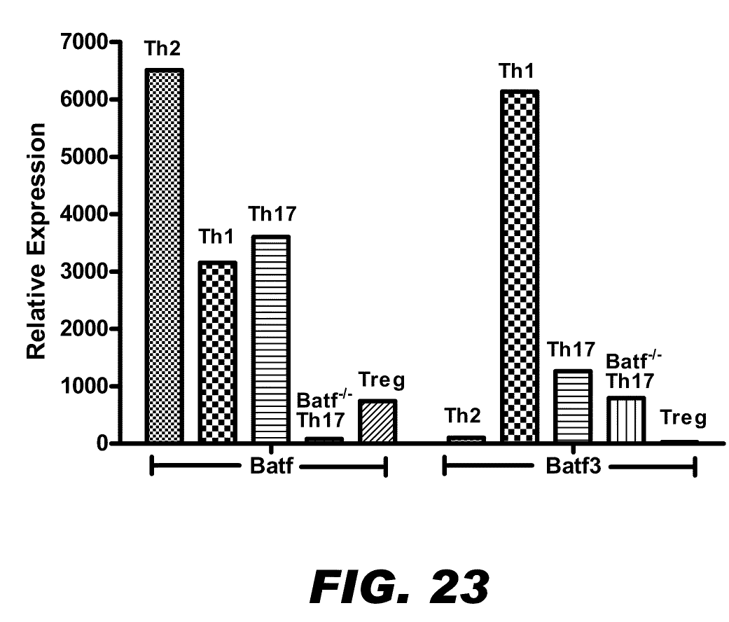

FIG. 23 depicts the relative expression of mouse Batf and Batf3 among T helper subsets determined using Affymetrix microarray.

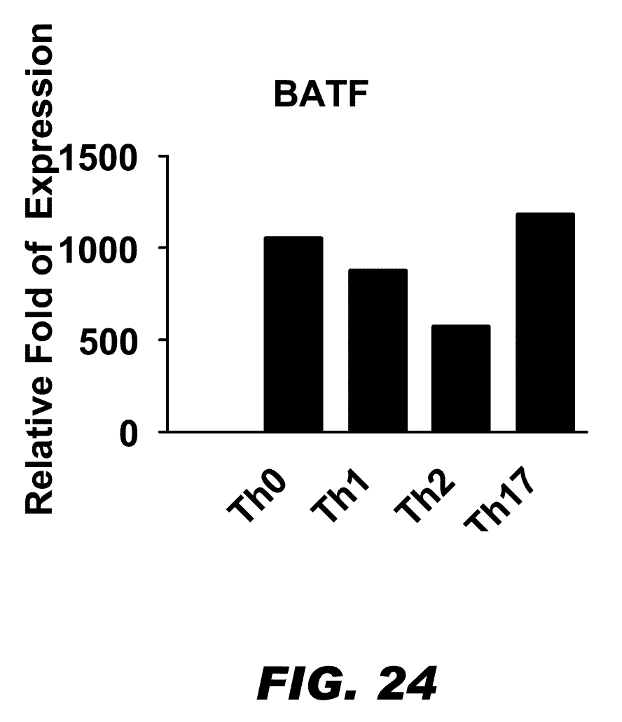

FIG. 24 depicts a plot showing the expression of human BATF among T helper subsets derived from human cord blood.

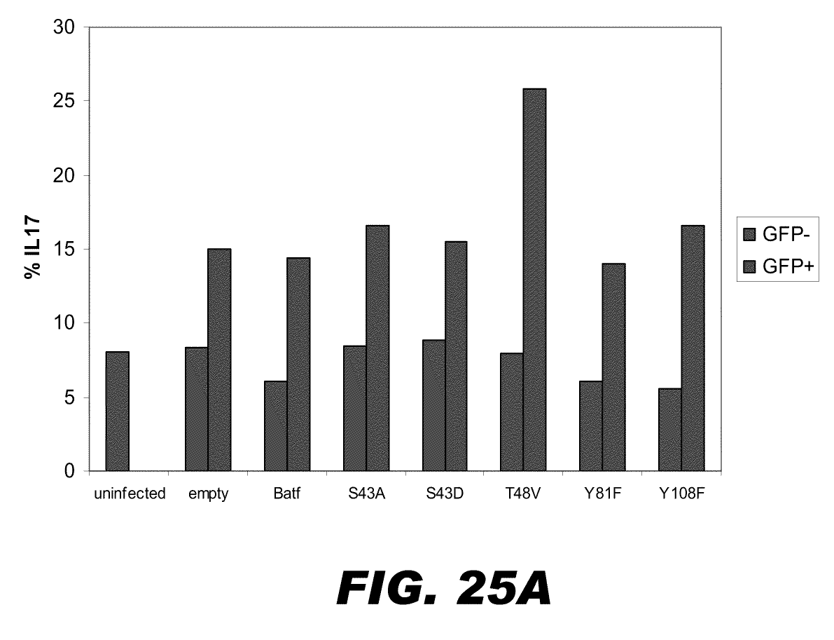

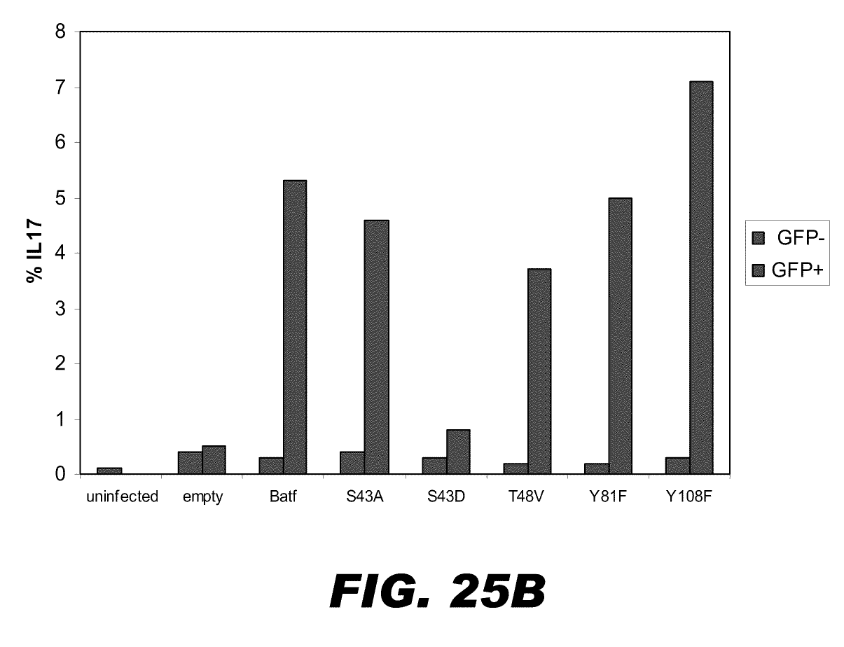

FIG. 25 depicts the effects of several Batf mutations on IL-17 production (a) day 6 wild-type, (b) day 6 Batf^{-/-}Batf3^{-/-} double knockout.

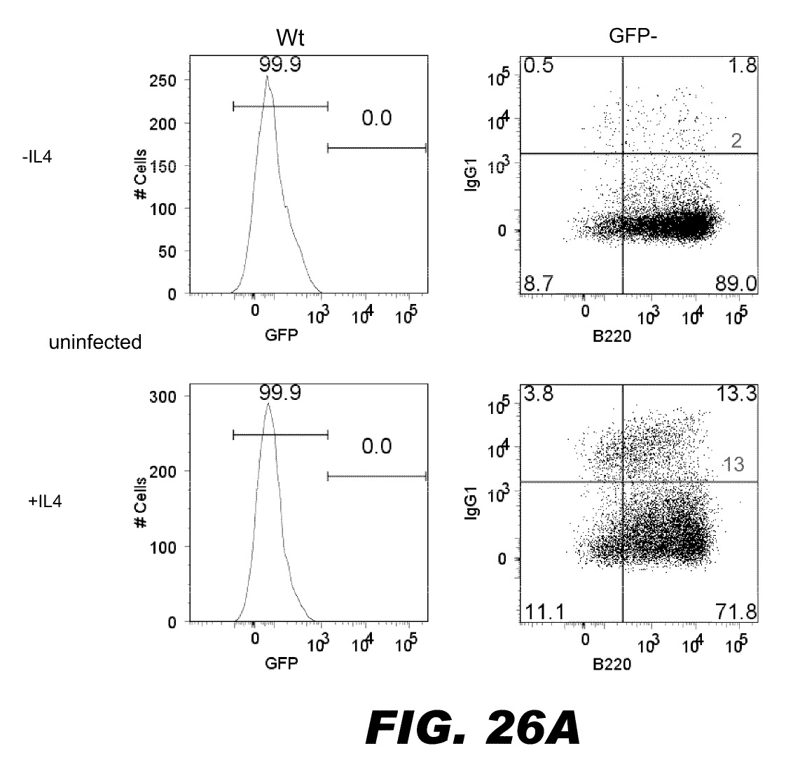

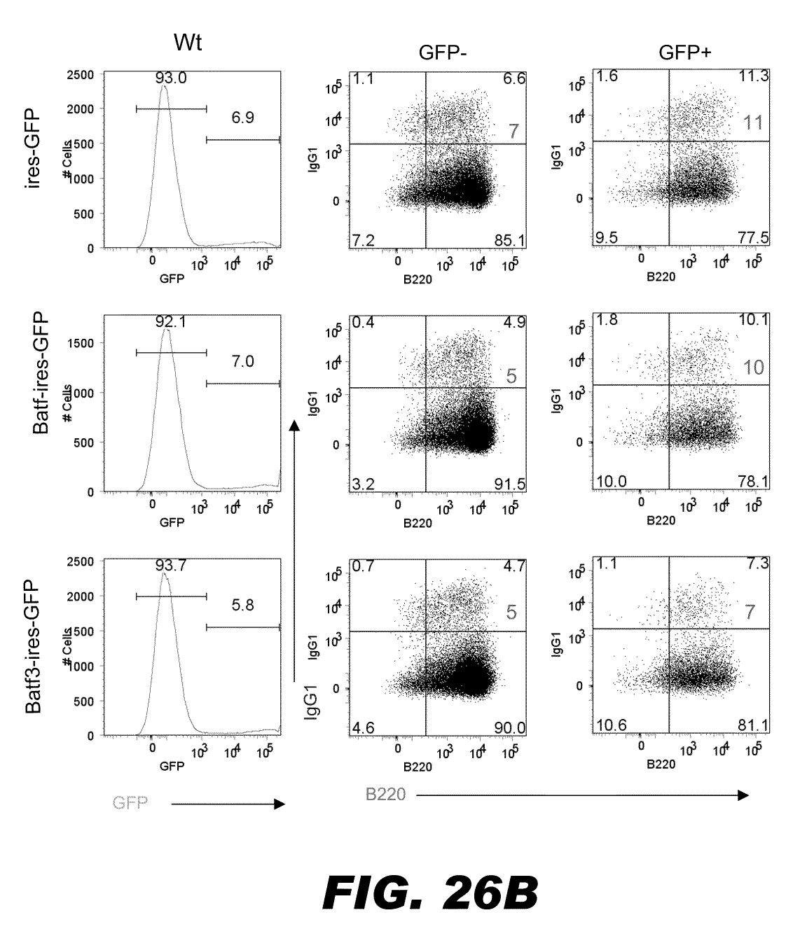

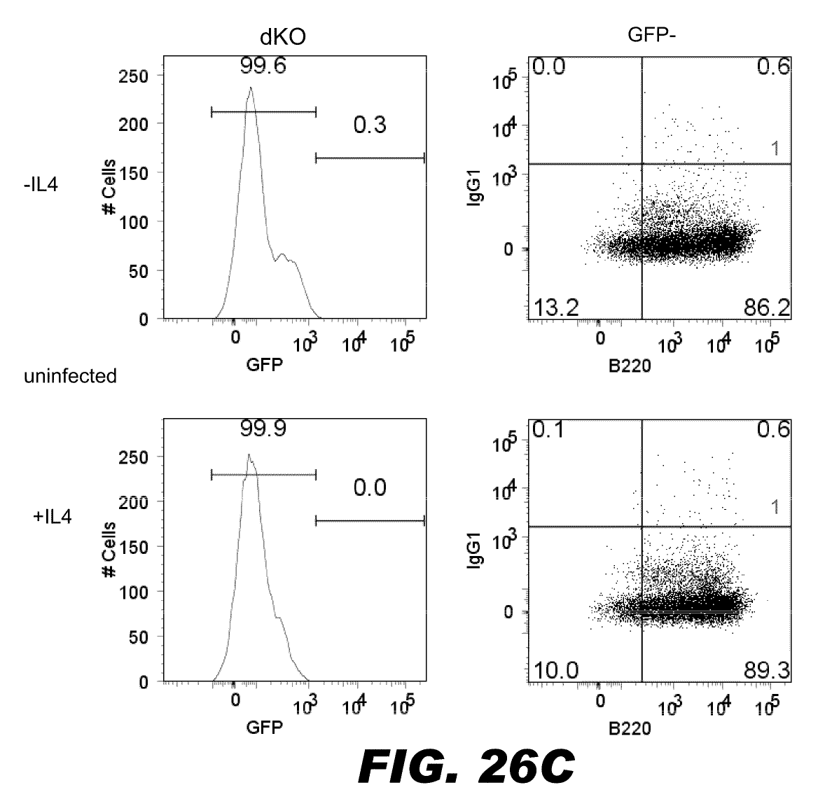

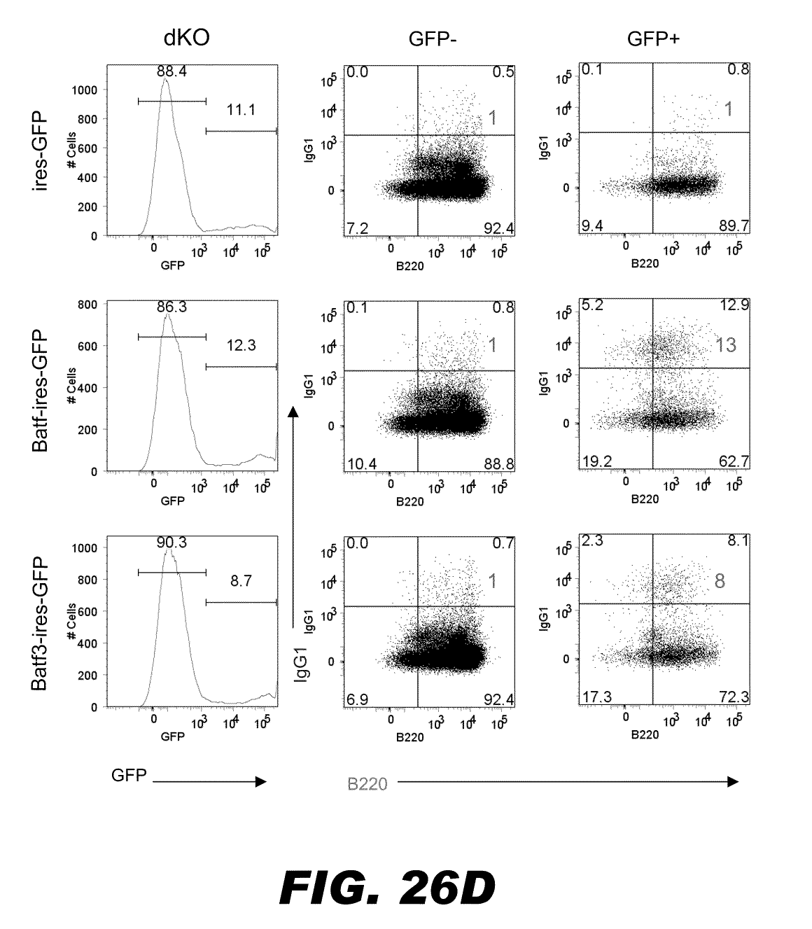

FIG. 26 depicts the effect of Batf and Batf3 on IL4 induced IgG1 switching in wild-type (a and b) and Batf^{-/-}Batf3^{-/-} double knockout B cells (c and d).

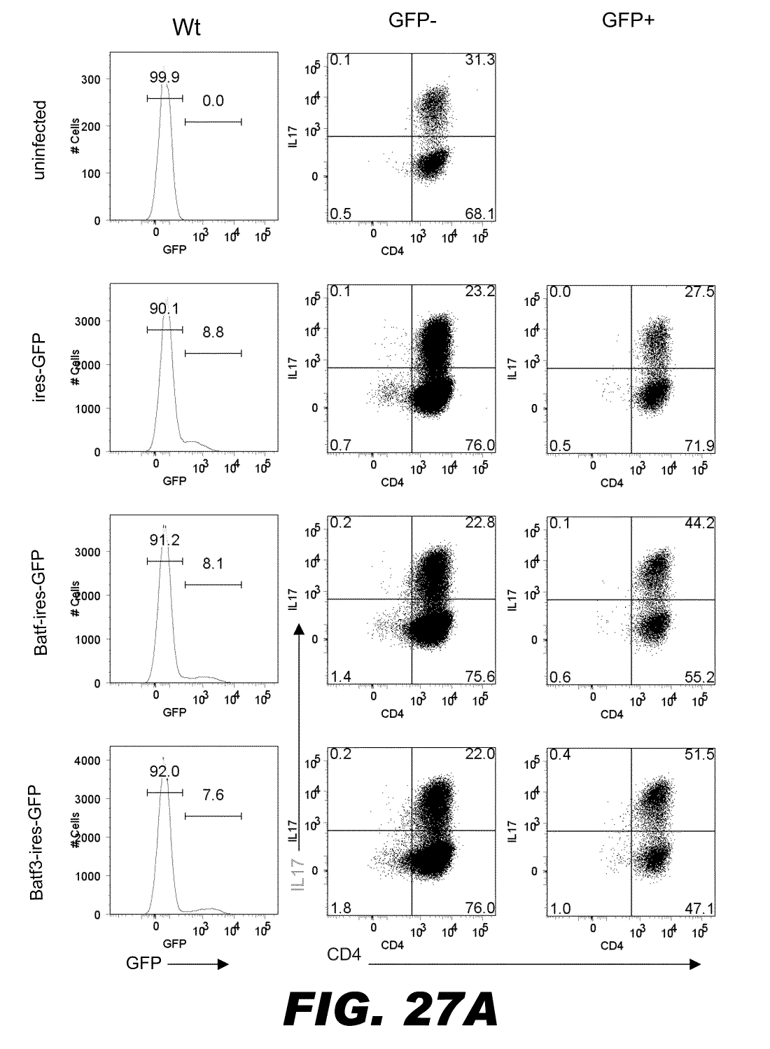

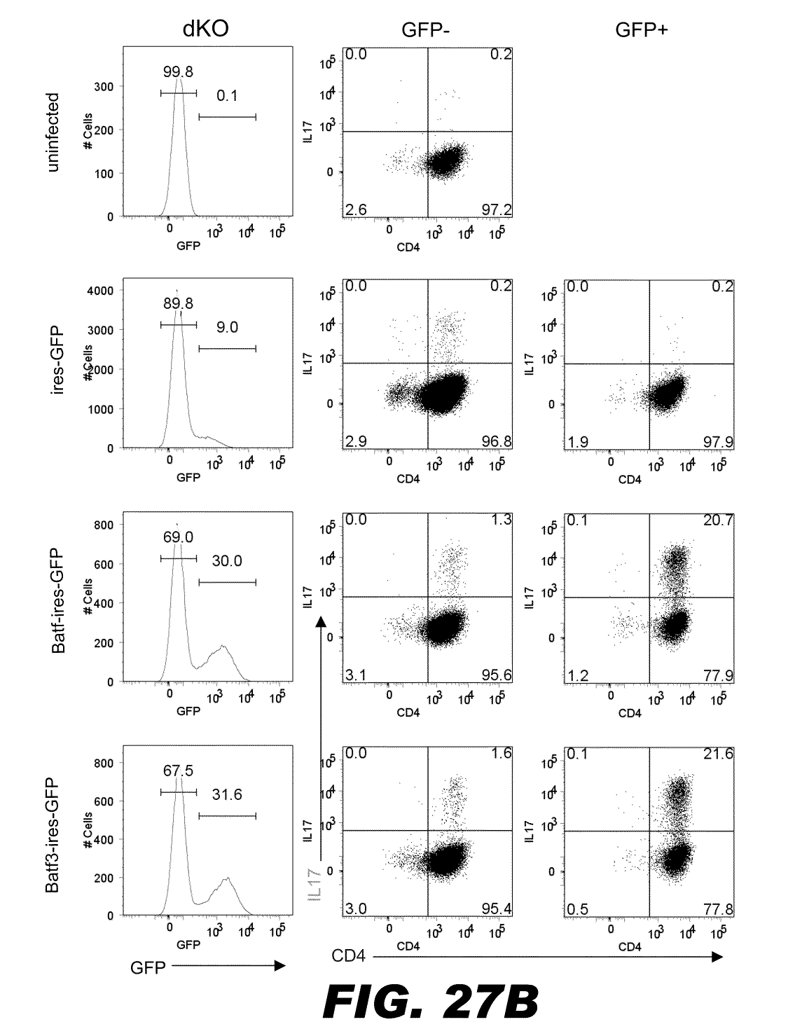

FIG. 27 depicts the effect of Batf and Batf3 on Th17 differentiation in wild-type (a) and Batf^{-/-}Batf3^{-/-} double knockout B cells (b).

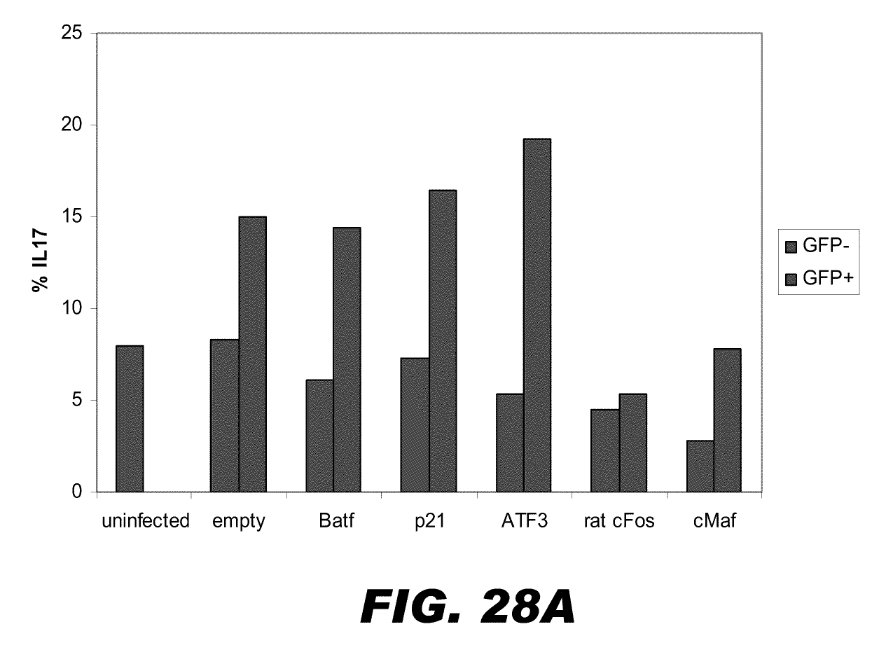

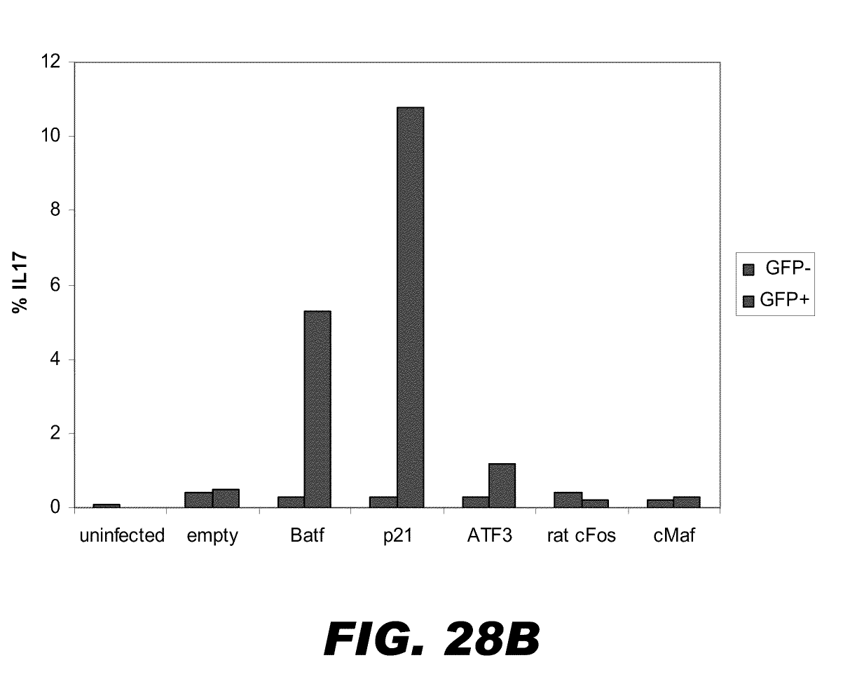

FIG. 28 depicts the effect of Batf and other bzip proteins on restoration of IL-17 production (a) day 6 wild-type, (b) day 6 Batf^{-/-}Batf3^{-/-} double knockout.

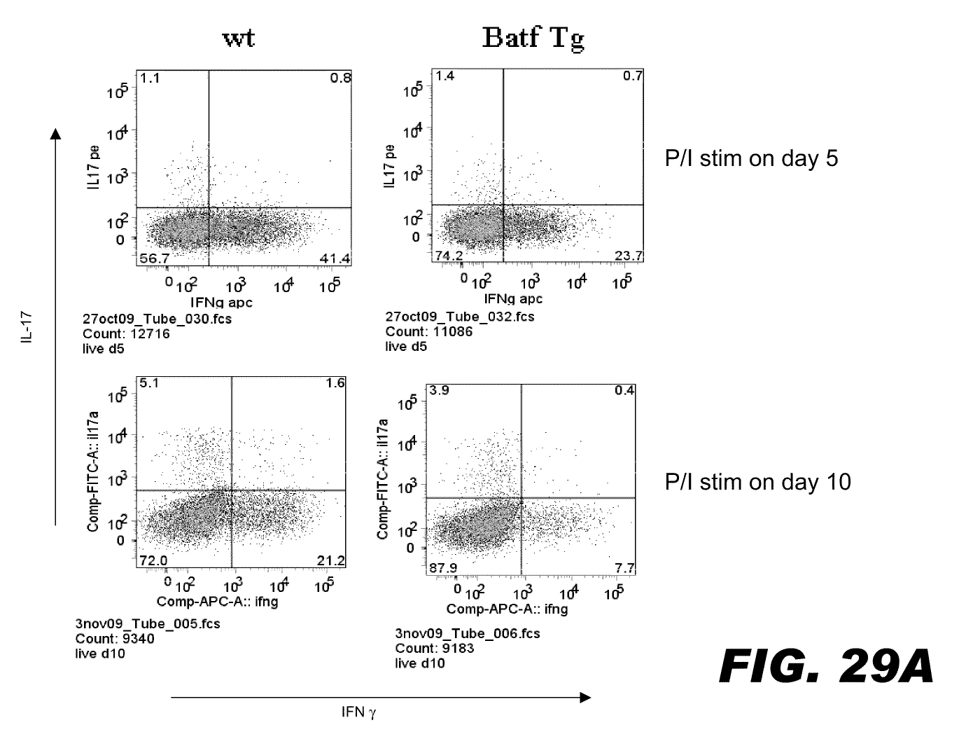

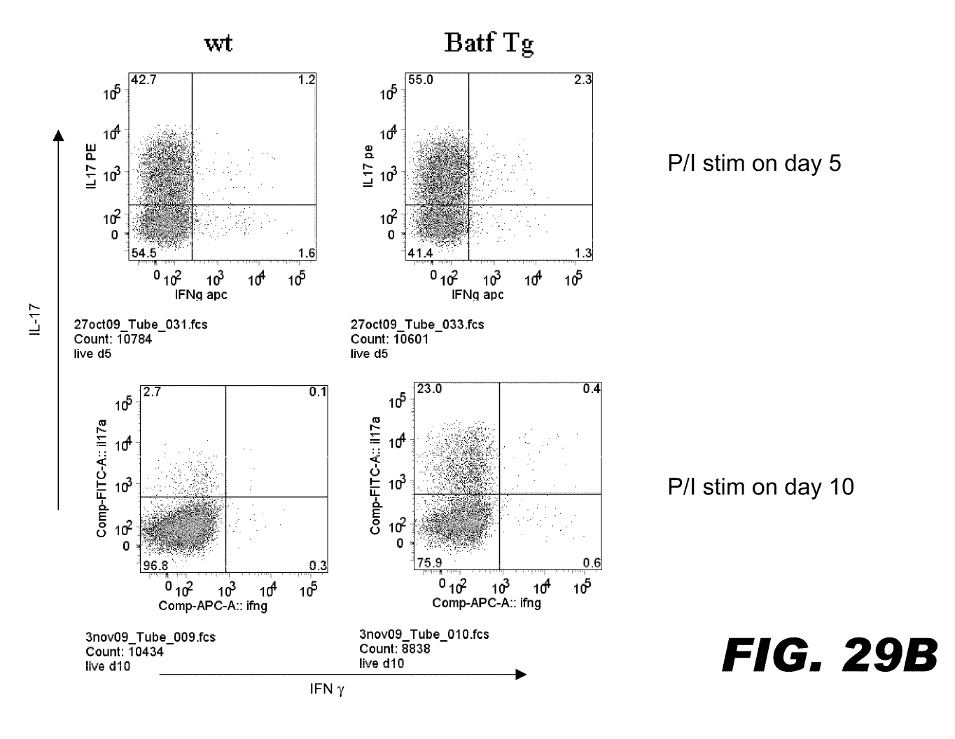

FIG. 29 depicts the effect of Batf expression of the ability to produce IL-17 (a) primary Th1 bulk D011.10 cultures, (b) primaryTh17 bulk D011.10 cultures.

### DETAILED DESCRIPTION OF THE INVENTION

The present invention encompasses a method to modulate the development of Th17 or Treg cells. As such, the present invention provides methods of modulating an immune response in a host. In particular, the present invention provides a nucleic acid sequence that modulates the development of Th17 or Treg cells.

### I. Nucleic Acid Sequence

In one aspect, the present invention encompasses a nucleic acid sequence that Batf or Batf3 is capable of binding (“Batf binding site”). In some embodiments, the Batf binding site may be 20, 15, 10, 8, 7, 6, 5, 4, or 3 nucleotides long. In preferred embodiments, the Batf binding site may be 10, 9, 8, 7, 6, 5 or 4 nucleotides long. Binding of Batf or Batf3 to the Batf binding site initiates or increases transcription of a nucleic acid sequence operably linked to the Batf binding site. In an exemplary embodiment, the Batf binding site may be 7 nucleotides long. In some embodiments, the sequence of the Batf binding site may be WKHBDVT, wherein the letters represent the nucleotide codes assigned by the International Union of Biochemistry (IUB) Nomenclature Committee. In certain embodiments, the sequence of the Batf binding site may be a sequence in Table A. As Batf or Batf3 may have a preference for the different binding sites encoded by the sequence, sequences may be tailored to bind Batf or Batf3 at the desired strength to tailor the desired response. By way of non-limiting example, binding of Batf to the Batf binding site in the IL-17 promoter increases transcription of IL-17. For more details, see the examples.

TABLE A
Batf binding sites

| | | | | | | |
|---|---|---|---|---|---|---|
| A | G | A | G | G | G | T |
| A | G | A | G | G | A | T |
| A | G | A | G | G | C | T |
| A | G | A | G | A | G | T |
| A | G | A | G | A | A | T |
| A | G | A | G | A | C | T |
| A | G | A | G | T | G | T |
| A | G | A | G | T | A | T |
| A | G | A | G | T | C | T |
| A | G | A | C | G | G | T |
| A | G | A | C | G | A | T |
| A | G | A | C | G | C | T |
| A | G | A | C | A | G | T |
| A | G | A | C | A | A | T |
| A | G | A | C | A | C | T |
| A | G | A | C | T | G | T |
| A | G | A | C | T | A | T |
| A | G | A | C | T | C | T |
| A | G | A | T | G | G | T |
| A | G | A | T | G | A | T |
| A | G | A | T | G | C | T |
| A | G | A | T | A | G | T |
| A | G | A | T | A | A | T |
| A | G | A | T | A | C | T |
| A | G | A | T | T | G | T |
| A | G | A | T | T | A | T |
| A | G | A | T | T | C | T |
| A | G | T | G | G | G | T |
| A | G | T | G | G | A | T |
| A | G | T | G | G | C | T |
| A | G | T | G | A | G | T |
| A | G | T | G | A | A | T |
| A | G | T | G | A | C | T |
| A | G | T | G | T | G | T |
| A | G | T | G | T | A | T |
| A | G | T | G | T | C | T |
| A | G | T | C | G | G | T |
| A | G | T | C | G | A | T |
| A | G | T | C | G | C | T |
| A | G | T | C | A | G | T |
| A | G | T | C | A | A | T |
| A | G | T | C | A | C | T |
| A | G | T | C | T | G | T |
| A | G | T | C | T | A | T |
| A | G | T | C | T | C | T |
| A | G | T | T | G | G | T |
| A | G | T | T | G | A | T |
| A | G | T | T | G | C | T |
| A | G | T | T | A | G | T |
| A | G | T | T | A | A | T |
| A | G | T | T | A | C | T |
| A | G | T | T | T | G | T |
| A | G | T | T | T | A | T |
| A | G | T | T | T | C | T |
| A | G | C | G | G | G | T |
| A | G | C | G | G | A | T |
| A | G | C | G | G | C | T |
| A | G | C | G | A | G | T |
| A | G | C | G | A | A | T |
| A | G | C | G | A | C | T |
| A | G | C | G | T | G | T |
| A | G | C | G | T | A | T |
| A | G | C | G | T | C | T |
| A | G | C | C | G | G | T |
| A | G | C | C | G | A | T |
| A | G | C | C | G | C | T |
| A | G | C | C | A | G | T |
| A | G | C | C | A | A | T |
| A | G | C | C | A | C | T |
| A | G | C | C | T | G | T |
| A | G | C | C | T | A | T |
| A | G | C | C | T | C | T |
| A | G | C | T | G | G | T |
| A | G | C | T | G | A | T |
| A | G | C | T | G | C | T |
| A | G | C | T | A | G | T |
| A | G | C | T | A | A | T |
| A | G | C | T | A | C | T |
| A | G | C | T | T | G | T |
| A | G | C | T | T | A | T |
| A | G | C | T | T | C | T |
| A | T | A | G | G | G | T |
| A | T | A | G | G | A | T |
| A | T | A | G | G | C | T |
| A | T | A | G | A | G | T |
| A | T | A | G | A | A | T |
| A | T | A | G | A | C | T |
| A | T | A | G | T | G | T |
| A | T | A | G | T | A | T |
| A | T | A | G | T | C | T |
| A | T | A | C | G | G | T |
| A | T | A | C | G | A | T |
| A | T | A | C | G | C | T |
| A | T | A | C | A | G | T |
| A | T | A | C | A | A | T |
| A | T | A | C | A | C | T |
| A | T | A | C | T | G | T |
| A | T | A | C | T | A | T |
| A | T | A | C | T | C | T |
| A | T | A | T | G | G | T |
| A | T | A | T | G | A | T |
| A | T | A | T | G | C | T |
| A | T | A | T | A | G | T |
| A | T | A | T | A | A | T |
| A | T | A | T | A | C | T |
| A | T | A | T | T | G | T |
| A | T | A | T | T | A | T |
| A | T | A | T | T | C | T |
| A | T | T | G | G | G | T |
| A | T | T | G | G | A | T |
| A | T | T | G | G | C | T |
| A | T | T | G | A | G | T |
| A | T | T | G | A | A | T |
| A | T | T | G | A | C | T |
| A | T | T | G | T | G | T |
| A | T | T | G | T | A | T |
| A | T | T | G | T | C | T |
| A | T | T | C | G | G | T |
| A | T | T | C | G | A | T |
| A | T | T | C | G | C | T |
| A | T | T | C | A | G | T |
| A | T | T | C | A | A | T |
| A | T | T | C | A | C | T |
| A | T | T | C | T | G | T |
| A | T | T | C | T | A | T |
| A | T | T | C | T | C | T |
| A | T | T | T | G | G | T |
| A | T | T | T | G | A | T |
| A | T | T | T | G | C | T |
| A | T | T | T | A | G | T |
| A | T | T | T | A | A | T |
| A | T | T | T | A | C | T |
| A | T | T | T | T | G | T |
| A | T | T | T | T | A | T |
| A | T | T | T | T | C | T |
| A | T | C | G | G | G | T |
| A | T | C | G | G | A | T |
| A | T | C | G | G | C | T |
| A | T | C | G | A | G | T |
| A | T | C | G | A | A | T |
| A | T | C | G | A | C | T |
| A | T | C | G | T | G | T |
| A | T | C | G | T | A | T |
| A | T | C | G | T | C | T |
| A | T | C | C | G | G | T |
| A | T | C | C | G | A | T |
| A | T | C | C | G | C | T |
| A | T | C | C | A | G | T |
| A | T | C | C | A | A | T |
| A | T | C | C | A | C | T |
| A | T | C | C | T | G | T |
| A | T | C | C | T | A | T |
| A | T | C | C | T | C | T |
| A | T | C | T | G | G | T |
| A | T | C | T | G | A | T |
| A | T | C | T | G | C | T |
| A | T | C | T | A | G | T |
| A | T | C | T | A | A | T |
| A | T | C | T | A | C | T |
| A | T | C | T | T | G | T |
| A | T | C | T | T | A | T |
| A | T | C | T | T | C | T |
| T | G | A | G | G | G | T |
| T | G | A | G | G | A | T |
| T | G | A | G | G | C | T |
| T | G | A | G | A | G | T |
| T | G | A | G | A | A | T |
| T | G | A | G | A | C | T |
| T | G | A | G | T | G | T |
| T | G | A | G | T | A | T |
| T | G | A | G | T | C | T |
| T | G | A | C | G | G | T |
| T | G | A | C | G | A | T |
| T | G | A | C | G | C | T |
| T | G | A | C | A | G | T |
| T | G | A | C | A | A | T |
| T | G | A | C | A | C | T |
| T | G | A | C | T | G | T |
| T | G | A | C | T | A | T |
| T | G | A | C | T | C | T |
| T | G | A | T | G | G | T |
| T | G | A | T | G | A | T |
| T | G | A | T | G | C | T |
| T | G | A | T | A | G | T |
| T | G | A | T | A | A | T |
| T | G | A | T | A | C | T |
| T | G | A | T | T | G | T |
| T | G | A | T | T | A | T |
| T | G | A | T | T | C | T |
| T | G | T | G | G | G | T |
| T | G | T | G | G | A | T |
| T | G | T | G | G | C | T |
| T | G | T | G | A | G | T |
| T | G | T | G | A | A | T |
| T | G | T | G | A | C | T |
| T | G | T | G | T | G | T |
| T | G | T | G | T | A | T |
| T | G | T | G | T | C | T |
| T | G | T | C | G | G | T |
| T | G | T | C | G | A | T |
| T | G | T | C | G | C | T |
| T | G | T | C | A | G | T |
| T | G | T | C | A | A | T |
| T | G | T | C | A | C | T |
| T | G | T | C | T | G | T |
| T | G | T | C | T | A | T |
| T | G | T | C | T | C | T |
| T | G | T | T | G | G | T |
| T | G | T | T | G | A | T |
| T | G | T | T | G | C | T |
| T | G | T | T | A | G | T |
| T | G | T | T | A | A | T |
| T | G | T | T | A | C | T |
| T | G | T | T | T | G | T |
| T | G | T | T | T | A | T |
| T | G | T | T | T | C | T |
| T | G | C | G | G | G | T |
| T | G | C | G | G | A | T |
| T | G | C | G | G | C | T |
| T | G | C | G | A | G | T |
| T | G | C | G | A | A | T |
| T | G | C | G | A | C | T |
| T | G | C | G | T | G | T |
| T | G | C | G | T | A | T |
| T | G | C | G | T | C | T |
| T | G | C | C | G | G | T |
| T | G | C | C | G | A | T |
| T | G | C | C | G | C | T |
| T | G | C | C | A | G | T |
| T | G | C | C | A | A | T |
| T | G | C | C | A | C | T |
| T | G | C | C | T | G | T |
| T | G | C | C | T | A | T |
| T | G | C | C | T | C | T |
| T | G | C | T | G | G | T |
| T | G | C | T | G | A | T |
| T | G | C | T | G | C | T |
| T | G | C | T | A | G | T |
| T | G | C | T | A | A | T |
| T | G | C | T | A | C | T |
| T | G | C | T | T | G | T |
| T | G | C | T | T | A | T |
| T | G | C | T | T | C | T |
| T | T | A | G | G | G | T |
| T | T | A | G | G | A | T |
| T | T | A | G | G | C | T |
| T | T | A | G | A | G | T |
| T | T | A | G | A | A | T |
| T | T | A | G | A | C | T |
| T | T | A | G | T | G | T |
| T | T | A | G | T | A | T |
| T | T | A | G | T | C | T |
| T | T | A | C | G | G | T |
| T | T | A | C | G | A | T |
| T | T | A | C | G | C | T |
| T | T | A | C | A | G | T |
| T | T | A | C | A | A | T |
| T | T | A | C | A | C | T |
| T | T | A | C | T | G | T |
| T | T | A | C | T | A | T |
| T | T | A | C | T | C | T |
| T | T | A | T | G | G | T |
| T | T | A | T | G | A | T |
| T | T | A | T | G | C | T |
| T | T | A | T | A | G | T |
| T | T | A | T | A | A | T |
| T | T | A | T | A | C | T |
| T | T | A | T | T | G | T |
| T | T | A | T | T | A | T |
| T | T | A | T | T | C | T |
| T | T | T | G | G | G | T |
| T | T | T | G | G | A | T |
| T | T | T | G | G | C | T |
| T | T | T | G | A | G | T |
| T | T | T | G | A | A | T |
| T | T | T | G | A | C | T |
| T | T | T | G | T | G | T |
| T | T | T | G | T | A | T |
| T | T | T | G | T | C | T |
| T | T | T | C | G | G | T |
| T | T | T | C | G | A | T |
| T | T | T | C | G | C | T |
| T | T | T | C | A | G | T |
| T | T | T | C | A | A | T |
| T | T | T | C | A | C | T |
| T | T | T | C | T | G | T |
| T | T | T | C | T | A | T |
| T | T | T | C | T | C | T |
| T | T | T | T | G | G | T |
| T | T | T | T | G | A | T |
| T | T | T | T | G | C | T |
| T | T | T | T | A | G | T |
| T | T | T | T | A | A | T |
| T | T | T | T | A | C | T |
| T | T | T | T | T | G | T |
| T | T | T | T | T | A | T |
| T | T | T | T | T | C | T |
| T | T | C | G | G | G | T |
| T | T | C | G | G | A | T |
| T | T | C | G | G | C | T |
| T | T | C | G | A | G | T |
| T | T | C | G | A | A | T |
| T | T | C | G | A | C | T |
| T | T | C | G | T | G | T |
| T | T | C | G | T | A | T |
| T | T | C | G | T | C | T |
| T | T | C | C | G | G | T |
| T | T | C | C | G | A | T |
| T | T | C | C | G | C | T |
| T | T | C | C | A | G | T |
| T | T | C | C | A | A | T |
| T | T | C | C | A | C | T |
| T | T | C | C | T | G | T |
| T | T | C | C | T | A | T |
| T | T | C | C | T | C | T |
| T | T | C | T | G | G | T |
| T | T | C | T | G | A | T |
| T | T | C | T | G | C | T |
| T | T | C | T | A | G | T |
| T | T | C | T | A | A | T |
| T | T | C | T | A | C | T |
| T | T | C | T | T | G | T |
| T | T | C | T | T | A | T |
| T | T | C | T | T | C | T |

In one embodiment of the invention, the Batf binding site may be operably linked to a nucleic acid sequence. For instance, in some embodiments, the Batf binding site may be operably linked to a promoter. A promoter may be positioned 5′ (upstream) or 3′ (downstream) of a nucleic acid sequence under its control. The distance between the promoter and a nucleic acid sequence may be approximately the same as the distance between that promoter and the native nucleic acid sequence it controls. In some embodiments, the Batf binding site may be operably linked to a natural promoter nucleic acid sequence in the cell. In other embodiments, the Batf binding site may be operably linked to a promoter derived from sources including viral, bacterial, fungal, plants, insects, and animals. A promoter may regulate the expression of a nucleic acid component constitutively, or differentially with respect to the cell, the tissue, or the organ in which expression occurs or, with respect to the developmental stage at which expression occurs, or in response to external stimuli such as physiological stresses, pathogens, metal ions, or inducing agents (i.e. an inducible promoter). Non-limiting representative examples of promoters may include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter. Additionally, the promoter may be a CMV immediate early promoter/enhancer (pCMV) or the CMV enhancer/chicken β-actin promoter (pCAG).

The Batf binding site may also be operably linked to a reporter nucleic acid sequence. Non-limiting examples of suitable reporter proteins may include a fluorescent protein (e.g., green fluorescent protein, red fluorescent protein, and the like), a luciferase, alkaline phosphatase, beta-galactosidase, beta-lactamase, horseradish peroxidase, or variants thereof. Other examples of reporter nucleic acid sequences are known in the art.

### (a) Transgenic Cells

In certain embodiments of the invention, the Batf binding site may be introduced into cells. The nucleic acid may be delivered to the cell using a viral vector or via a non-viral method of transfer. Viral vectors suitable for introducing nucleic acids into cells may include retroviruses, adenoviruses, adeno-associated viruses, rhabdoviruses, and herpes viruses. Non-viral methods of nucleic acid transfer may include naked nucleic acid, liposomes, and protein/nucleic acid conjugates. The exogenous nucleic acid that is introduced to the cell may be linear or circular, may be single-stranded or double-stranded, and may be DNA, RNA, or any modification or combination thereof.

In general, the exogenous nucleic acids are introduced into the eukaryotic cells by transfection. Methods for transfecting nucleic acids are well known to persons skilled in the art. Transfection methods may include, but are not limited to, viral transduction, cationic transfection, liposome transfection, dendrimer transfection, electroporation, heat shock, nucleofection transfection, magnetofection, nanoparticles, biolistic particle delivery (gene gun), and proprietary transfection reagents such as Lipofectamine, Dojindo Hilymax, Fugene, jetPEI, Effectene, or DreamFect.

Upon introduction to the cell, the exogenous nucleic acid may be integrated into a chromosome. In some embodiments, integration of the exogenous nucleic acid into a cellular chromosome may be achieved with a mobile element. Non-limiting examples of a mobile element may include a transposon or a retroelement. A variety of transposons are suitable for use in the invention. Examples of DNA transposons that may be used include the Mu transposon, a P element transposon from Drosophila, and members of the Tc1/Mariner superfamily of transposons such as the sleeping beauty transposon from fish. A variety of retroelements may be suitable for use in the invention and may include LTR-containing retrotransposons and non-LTR retrotransposons. Non-limiting examples of retrotransposons may include Copia and gypsy from Drosophila melanogaster, the Ty elements from Saccharomyces cerevisiae, the long interspersed elements (LINEs), and the short interspersed elements (SINEs) from eukaryotes. Suitable examples of LINEs may include L1 from mammals and R2Bm from silkworm.

In other embodiments, integration of the exogenous nucleic acid into a cellular chromosome may be mediated by a virus. Viruses that integrate nucleic acids into a chromosome may include adeno-associated viruses and retroviruses. Adeno-associated virus (AAV) vectors may be from human or nonhuman primate AAV serotypes and variants thereof. Suitable adeno-associated viruses may include AAV type 1, AAV type 2, AAV type 3, AAV type 4, AAV type 5, AAV type 6, AAV type 7, AAV type 8, AAV type 9, AAV type 10, or AAV type 11. A variety of retroviruses may be suitable for use in the invention. Retroviral vectors may either be replication-competent or replication-defective. The retroviral vector may be an alpharetrovirus, a betaretrovirus, a gammaretrovirus, a deltaretrovirus, an epsilonretrovirus, a lentivirus, or a spumaretrovirus. In a preferred embodiment, the retroviral vector may be a lentiviral vector. The lentiviral vector may be derived from human, simian, feline, equine, bovine, or lentiviruses that infect other mammalian species. Non-limiting examples of suitable lentiviruses may include human immunodeficiency virus (HIV), simian immunodeficiency virus (SIV), feline immunodeficiency virus (FIV), bovine immunodeficiency virus (BIV), and equine infectious anemia virus (EIAV). In an exemplary embodiment, the lentiviral vector may be an HIV-derived vector.

Integration of the exogenous nucleic acid into a chromosome of the cell may be random. Alternatively, integration of the exogenous nucleic acid may be targeted to a particular sequence or location of a chromosome. Typically, the general environment at the site of integration may affect whether the integrated exogenous nucleic acid is expressed, as well as its level of expression.

In some embodiments, the cells may be derived from the digestive system, the skeletal system, the muscular system, the nervous system, the endocrine system, the respiratory system, the circulatory system, the reproductive system, the integumentary system, the lymphatic system, or the urinary system. In preferred embodiments, the sample may be derived from the lymphatic system. In a more preferred embodiment, the sample may be immune cells derived from the lymphatic system. In some embodiments, the immune cells derived from the lymphatic system may be neutrophils, eosinophils, basophils, lymphocytes, monocytes, macrophages, or progenitor cells that produce these cells. In preferred embodiments, the immune cells derived from the lymphatic system may be lymphocytes, such as T cells, B cells or natural killer (NK) cells or progenitor cells that produce lymphocytes. In preferred embodiments, the immune cells derived from the lymphatic system may be T cells.

Methods for purification or enrichment of certain cell types from a sample are well known in the art and are discussed in Ausubel et al., (2003) Current Protocols in Molecular Biology, John Wiley & Sons, New York, N.Y., or Sambrook et al. (1989) Molecular Cloning: A Laboratory Manual, Cold Spring Harbor Press, Cold Spring Harbor, N.Y. One skilled in the art will know which parameters may be manipulated to optimize purification or enrichment of cells of interest. Most commonly, cells are purified or enriched using immunoaffinity to antigens expressed on the surface of the cells. In short, the sample, consisting of a mixture of cells to be separated is incubated with a solid support, usually superparamagnetic beads that facilitate later steps. The solid support is coated with antibodies against a particular surface antigen, causes the cells expressing this antigen to attach to the solid support. If the solid support is superparamagnetic beads, the cells attached to the beads (expressing the antigen) can be separated from the sample by attraction to a strong magnetic field. The procedure may be used for positively selecting the cells expressing the antigen(s) of interest. In negative selection the antibody used is against surface antigen(s), which are known to be present on cells that are not of interest, therefore enriching the sample with the cells of interest.

### (b) Transgenic Animals

In some aspects, one or more of the nucleic acid sequences described above may be introduced into and stably expressed in an animal. For instance, transgenic mice may be generated using procedures well known to those of skill in the art. In some embodiments, the introduced nucleic acid sequence may be randomly integrated into the chromosome of the animal. In other embodiments, the nucleic acid sequence is integrated at a specific site in the chromosome of the animal. Suitable animals may include commonly used laboratory animals, such as rodents.

### II. Modulation of TH17 Cells

In some aspects, the invention provides for modulation of an immune response by modulating Th17 cell development.

### (a) Modulation of Batf

As demonstrated in the examples, modulating Batf or Batf3 expression may modulate the development of a Th17 cell. As used herein, the phrase “modulating Batf expression” refers to modulating the amount of Batf or Batf3 or the activity of Batf or Batf3. In certain embodiments, modulating Batf expression refers to modulating the amount of Batf or Batf3. In some embodiments, the amount of Batf or Batf3 may be increased. In other embodiments, the amount of Batf or Batf3 may be decreased. The amount of Batf or Batf3 may be modulated by modulating the expression of Batf or Batf3 respectively. Methods of modulating the expression of Batf may include modulating inducers of Batf or Batf3 expression. Non-limiting examples of Batf or Batf3 inducers may include STAT3, IL-6, leukemia inhibitory factor (LIF), and the EBV-encoded EBNA2. Batf expression may also be modulated by modulating expression of the Batf or Batf3 nucleic acid sequence at transcription or translation. For example, the nucleic acid sequence encoding the Batf or Batf3 polypeptide may be altered such that levels of functional messenger RNA (mRNA) (and, consequently, a functional polypeptide) are increased, decreased or not made. Alternatively, the mRNA may be altered such that levels of the polypeptide are increased, decreased or not made. Non-limiting examples of methods to modulate Batf or Batf3 transcription or translation may include RNA interference agents (RNAi) or gene targeting methods. Standard methods for modulating transcription or translation of a specific nucleic acid sequence are known to individuals skilled in the art. Guidance may be found in Current Protocols in Molecular Biology (Ausubel et al., John Wiley & Sons, New York, 2003) or Molecular Cloning: A Laboratory Manual (Sambrook & Russell, Cold Spring Harbor Press, Cold Spring Harbor, N.Y., 3rd edition, 2001).

In some embodiments, modulating Batf expression refers to modulating the activity of Batf or Batf3. As used herein, the phrase “modulating Batf or Batf3 activity” refers to modulating the activity of Batf or Batf3 by modulating the activity of the functional polypeptide complex containing Batf or Batf3. In some embodiments, modulating Batf or Batf3 activity may include modulating the activity of a Batf or Batf3 interaction partner. In other embodiments, modulating Batf or Batf3 activity may include modulating the level of Batf or Batf3 phosphorylation. Batf or Batf3 phosphorylation may be modulated by modulating Batf or Batf3 phosphorylation sites, for instance, serine 43, or by modulating the activity of kinases that phosphorylate Batf or Batf3. Batf or Batf3 activity may also be modulated by modulating Batf or Batf3 binding to the Batf binding site, or activation or transcription of nucleic acids functionally linked to the Batf binding site. Modulating Batf or Batf3 activity may be with an agonist or antagonist. An agonist or antagonist may be a molecule that inhibits or attenuates the biological activity of a Batf or Batf3 polypeptide. Non-limiting examples of suitable antagonists or agonists may include natural compounds, synthetic compounds, small organic compounds, nucleic acids, carbohydrates, peptides, peptide nucleic acids, peptidomimetics, antibodies, antisense oligonucleotides, or aptamer oligonucleotides. In one embodiment, a suitable antagonist or agonist may be an antibody. In another embodiment, a suitable antagonist or agonist may be a small molecule inhibitor. Batf or Batf3 activity may also be modulated by altering Batf or Batf3. For example, Batf or Batf3 may be altered by changing the number or sequence of phosphorylation sites on Batf or Batf3, altering the nucleic acid binding ability of Batf or Batf3, or altering the ability of Batf or Batf3 to interact with other polypeptides.

### (b) Modulation of Batf-Dependent Nucleic Acids

A microarray study comparing the nucleic acid expression of activated Batf^{+/+} and Batf^{-/-}T cells revealed 110 nucleic acid sequences whose expression is highly dependent on Batf (Table 2). Modulating these Batf-dependent nucleic acids may modulate Th17 cell development. Therefore, in some embodiments, Th17 development may be modulated by modulating a nucleic acid sequence of Table 2. In a preferred embodiment, Th17 development may be modulated by modulating RORγt. In another preferred embodiment, Th17 development may be modulated by modulating RORα. In yet another preferred embodiment, Th17 development may be modulated by modulating the aryl hydrocarbon receptor (AHR). In another preferred embodiment, Th17 development may be modulated by modulating IL-22. In still another preferred embodiment, Th17 development may be modulated by modulating IL-17. In an additional preferred embodiment, Th17 development may be modulated by modulating DLGH2. In some embodiments, Th17 cell numbers may be modulated by modulating one or more of the sequences of Table 2. This may be done using standard pharmacotherapeutic techniques described above.

### (c) Cell Therapy

In some aspects of the invention, cell therapy techniques may be appropriate for modulating an immune response. Generally speaking, cell therapy describes the introduction of new cells into a tissue in order to treat a disease. As applied to the invention, immune cells may be harvested from a subject and modified as described above, and then reintroduced into the subject using techniques known in the art.

### III. Methods for Modulating an Immune Response

Yet another aspect of the present invention encompasses methods for modulating an immune response. In some embodiments, the immune response may be an autoimmune response. In other embodiments, the immune response may be an anti-tumor immune response. In certain embodiments, the immune response may be against a pathogen. In each of the above embodiments, the method comprises modulating Th17 cells, as described in section II above.

### (a) Autoimmune Response

In one embodiment, the invention encompasses a method for modulating an autoimmune response. Generally speaking, the method comprises modulating Th17 cells, as described above. In particular, the method may comprise decreasing the development of Th17 cells. Non-limiting examples of autoimmune responses may include: acute disseminated encephalomyelitis (ADEM), Addison's disease, ankylosing spondylitis, antiphospholipid antibody syndrome (APS), autoimmune hemolytic anemia, autoimmune hepatitis, bullous pemphigoid, coeliac disease, dermatomyositis, diabetes mellitus type 1, goodpasture's syndrome, graves' disease, Guillain-Barré syndrome (GBS), Hashimoto's disease, idiopathic thrombocytopenic purpura, Lupus erythematosus, multiple sclerosis, myasthenia gravis, pemphigus vulgaris, pernicious anaemia, polymyositis, primary biliary cirrhosis, rheumatoid arthritis, Sjögren's syndrome, temporal arteritis (also known as “giant cell arteritis”), vasculitis, and Wegener's granulomatosis.

In particular embodiments, the automimmune response may be response against a transplanted organ. In other embodiments, the automimmune response may be a graft vs. host response.

### (b) Immune Response Against Pathogens

In another embodiment, the invention encompasses a method for modulating an immune response against a pathogen. Typically, the method comprises modulating Th17 cells, as described above. During an immune response against a pathogen, Th17 cells promote inflammation and attract neutrophils. Hence, in a preferred embodiment, modulation of Th17 development may result in an increase in Th17 cell development.

Methods of modulating Th17 development are described above.

### (c) Immune Response Against a Tumor

In yet another embodiment, the invention provides a method for modulating an anti-tumor immune response. The method generally comprises modulating Th17 development, as described above. Non-limiting examples of cancers that may be targeted by the invention, classified by the type of cell that resembles the tumor and, therefore, the tissue presumed to be the origin of the tumor may be a carcinoma such as breast, prostate, lung and colon cancer; a sarcoma such as bone cancer; lymphoma and leukemia; germ cell tumors such as testicular cancer; or blastic tumor or blastoma.

### IV. Methods of Screening for Modulators of Batf

A further aspect of the invention provides a method to screen for modulators of Batf or Batf3. Typically, the method relies on Batf or Batf3 properties described in the invention, including binding of Batf or Batf3 to the Batf binding site and activation of transcription of nucleic acid sequences downstream of the binding sequence.

In some embodiments, screening for modulators of Batf or Batf3 may be performed in vitro by screening for modulators of Batf or Batf3 binding to the Batf binding site. Generally, these methods entail contacting a mixture of Batf or Batf3 and a nucleic acid containing the Batf binding site with a compound, and then measuring the binding.

In other embodiments, screening for modulators of Batf or Batf3 may be in a cell-based assay. In some embodiments, Batf or Batf3 activity may be measured by measuring expression of a nucleic acid target of Batf or Batf3. In other embodiments, Batf or Batf3 activity may be measured by measuring expression of a reporter nucleic acid controlled by Batf or Batf3 and introduced into cells or animals as described in section I. In such an assay, cells may be contacted with the compound and the activity of Batf or Batf3 may be measured by measuring expression of the nucleic acid controlled by the Batf binding site. Methods of measuring nucleic acid expression are known to a person skilled in the art. As Batf functions as part of a complex with other cellular polypeptides, these methods may identify compounds that inhibit Bat or Batf3f, another polypeptide required for the function of the Bat or Batf3f-containing complex, or the interaction of Batf or Batf3 with one of its partners.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Th17 cells” refers to a discrete population of CD4+ helper T cells that has been described as the predominant source of IL-17. These cells have been named Th17 cells.

“Th17 cell development” refers to the cellular differentiation necessary for the development of a Th17 cell. A Th17 cell is ‘developed’ if it produces IL-17.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification of Transcription Factors Selectively Expressed in Various Effector T Cell Subsets

A global survey of gene expression was used to identify transcription factors selectively expressed in various effector T cell subsets (FIG. 1a). This survey identified the B cell activating transcription factor (Batf) as highly expressed in effector TH1, TH2 and TH17 cells, expressed at lower levels in naïve T cells and B cells and at essentially basal levels in other tissues. Batf is a member of the bZIP family and forms heterodimers with Jun. Some AP-1 proteins, including Batf and the related Snft6, are composed only of a basic region and leucine zipper and lack a transactional activation domain (TAD). Batf and Snft can each inhibit AP-1 dependent transcriptional activity and have been thought to function as endogenous repressors of AP-1 activity.

### Example 2

### Effect of Batf on T Cell Differentiation in Mice

### Generation of Batf^{-/-} Mice

Since AP-1 regulates T cell differentiation and cytokine production, Batf^{-/-} mice were generated to assess its role in effector T cells (FIGS. 1b and c). Batf^{-/-} mice were born at normal Mendelian frequencies, were fertile, healthy and lacked detectable Batf protein (FIG. 1d).

### Characterization of Batf^{-/-} Mice

Batf^{-/-} mice had no abnormalities in thymic or spleen cellularity, lymph node development (FIG. 2), or in CD4+ and CD8+T cell development in thymus, spleen or lymph nodes (FIGS. 3a and b). Despite reported alteration of NKT cell development in Batf-transgenic mice, in this experiment NKT cell development in Batf^{-/-} mice was normal (FIG. 3c). Batf^{-/-} mice had normal B cell development (FIGS. 3d and e) and normal conventional and plasmacytoid dendritic cell development (FIGS. 4a and b).

### Results

Batf^{-/-} mice exhibited a remarkably selective defect in one particular pathway of T cell differentiation (FIG. 5). Batf^{-/-}T cells displayed normal TH1 and TH2 differentiation (FIG. 5a). Batf^{-/-}T cells activated under TH17 conditions, however, showed a dramatic loss in IL-17 production (FIG. 5b), but produced normal levels of IL-2 without compensatory changes in IFN-γ or IL-10. Batf^{-/-}T cells produced normal levels of IL-17 (FIG. 5c). Even after repeated rounds of activation under TH17 conditions, Batf^{-/-} D011.10 T cells showed dramatically reduced levels of IL-17 production (FIG. 6a). Interestingly, Batf^{-/-}CD8+T cells activated under TH17 conditions also showed a loss of IL-17 production (FIG. 6b).

### Example 3

### Overexpression of Batf in Mice

To examine Batf overexpression, transgenic mice expressing FLAG-tagged Batf under the control of the CD2 promoter were generated. Batf-transgenic D011.10 T cells and CD8+T cells produced increased IL-17 when activated under TH17 conditions compared to non-transgenic T cells (FIGS. 6c and d). Lamina propria CD4⁺T cells, which constitutively express IL-17 in wild type mice, failed to produce IL-17 in Batf^{-/-} mice (FIG. 6e). In summary, Batf^{-/-}T cells showed a uniform loss of IL-17 production.

### Example 4

### Batf^{-/-} Mice are Resistant to Experimental Autoimmune Encephalomyelitis

TH17 cells are the major pathogenic population in the model of experimental autoimmune encephalomyelitis (EAE). To test whether Batf^{-/-} mice were susceptible to EAE, we immunized Batf^{+/+} and Batf^{-/-} mice with myelin oligodendrocyte glycoprotein peptide 35-55 (MOG35-55) (FIG. 7). Eleven Batf^{+/+} mice (n=12) developed EAE with a mean maximum score of 3.7, whereas no Batf^{-/-} mice (n=13) developed any signs of disease within 40 days after immunization (FIG. 7a). CD4⁺T cells that infiltrated the CNS of Batf^{+/+} mice produced IL-17 and IFN-γ at peak disease, whereas the few CD4⁺T cells that infiltrated the CNS of Batf^{-/-} mice produced no IL-17, but made similar amounts of IFN-γ as Batf^{+/+}T cells (FIG. 7b). Prior to disease onset, CD4⁺T cells producing IL-17 were present in Batf^{+/+} spleens, but not Batf^{-/-} spleens (FIG. 8a). IL-6-deficient mice are resistant to EAE due to a compensatory increase in Foxp3⁺T regulatory (Treg) cells. Thus, resistance to EAE in Batf^{-/-} mice could conceivably result either from the loss of IL-17-producing effector T cells, or from an increase in Treg cells. We analyzed splenic T cells in Batf^{+/+} and Batf^{-/-} mice for Foxp3 expression 10 and 40 days after immunization with MOG35-55 (FIGS. 8b and c). Batf^{-/-} mice had lower baseline numbers of Foxp3⁺T cells in the spleen compared to Batf^{+/+} mice, but showed no change in Foxp3⁺ expression after MOG35-55 immunization (FIGS. 8b and c), suggesting that their resistance to EAE results from an absence of TH17 cells rather than an increase in Treg cells.

### Example 5

### Resistance to EAE is Due to a T Cell Intrinsic Defect

The loss of TH17 development in Batf^{-/-} mice could result either from a defect within T cells or a defect in antigen-presenting cells. To distinguish these possibilities, we carried out an adoptive transfer study by injecting naïve Batf^{+/+}CD4+T cells or a PBS control buffer into mice before MOG35-55 immunization (FIG. 7c). Batf^{-/-} mice receiving PBS control buffer remained resistant to EAE as expected. In contrast, Batf^{-/-} mice receiving naïve Batf^{+/+}CD4+T cells developed severe EAE (FIG. 7c, Table 1) and showed infiltration of the CNS by IL-17-producing CD4⁺T cells (FIG. 8d). These results indicate that the antigen-presenting environment in Batf^{-/-} mice is permissive for TH17 development, and suggest that resistance to EAE is due to a T cell intrinsic defect.

TABLE 1
Transfer of Batf+/+ CD4+ T cells into Batf−/− mice restores EAE

| | | Mean Max. | |
|---|---|---|---|
| Group | Incidence | Score | Mortality |
| PBS→Batf^{+/+} | 5 of 5 (100%) | 3.4 ± 0.7 | 1 of 5 (20%) |
| PBS→Batf^{-/-} | 0 of 5 (0%) | 0 | 0 of 13 (0%) |
| Batf^{+/+}CD4⁺→Batf^{+/+} | 5 of 6 (83%) | 3.0 ± 0.6 | 0 of 6 (0%) |
| Batf^{+/+}CD4⁺→Batf^{-/-} | 4 of 6 (66%) | 2.4 ± 1.0 | 2 of 6 (33%) |

Four days prior to induction of EAE mice were injected with 1 × 107 CD4⁺Batf^{+/+} T cells or control buffer (PBS) as indicated. The mice were monitored for disease development as described in Methods. Mean maximum score of disease was calculated and is presented ± s.e.m.

### Example 6

### Batf Required for Gene Induction Downstream of IL-6, IL-21 and Tgf-Beta

Batf could control TH17 development either by regulating the expression of components of the IL-6, IL-21 or TGF-β signaling pathways, or by regulating induction of their downstream target genes. Batf^{-/-}CD4⁺T cells showed normal levels of IL-6 receptor expression and IL-6-induced STAT3 phosphorylation (FIGS. 9a and b). Proximal IL-21 signaling was also intact, since Batf^{-/-} CD4⁺T cells showed normal levels of IL-21-induced STAT3 phosphorylation (FIG. 9c). Finally, proximal TGF-β signaling appeared intact based on normal induction of Foxp3 by TFG-beta in Batf^{-/-} CD4⁺T cells (FIG. 9d). Thus, proximal signaling of IL-6, IL-21 and TGF-β was intact in Batf^{-/-}T cells, suggesting that Batf may be required for induction of genes downstream of these pathways.

Consistently, induction of IL-21, an early target of IL-6 signaling in CD4⁺T cells18, was significantly reduced in Batf^{-/-} CD4⁺T cells activated under TH17 conditions (FIG. 10a). This reduction could potentially explain the absence of TH17 development in Batf^{-/-}T cells, since autocrine IL-21 is required for TH17 development. To test if reduced IL-21 is the only defect in Batf^{-/-}T cells, we supplemented TH17 differentiation conditions with IL-21. Addition of IL-21 failed to rescue TH17 development in Batf^{-/-}T cells (FIG. 10b), indicating that additional factors are controlled by Batf during TH17 differentiation.

### Example 7

### Identification of Additional Batf Targets

To identify additional Batf targets, we performed DNA microarrays and quantitative RT-PCR comparing gene expression of Batf^{+/+} and Batf^{-/-}T cells activated in the presence or absence of IL-6 and/or TGF-β (FIG. 10c, d). This analysis identified additional Batf-dependent genes, some of which were known to regulate TH17 development (FIG. 10c, d, and Table 2). Batf-dependent genes included RORγt, RORγt, the aryl hydrocarbon receptor (AHR)26-28, IL-22 and IL-17. In contrast, IRF-4 expression was unchanged in Batf^{-/-}T cells. Early induction of RORγt occurred normally in Batf^{-/-}T cells but RORγt expression was not maintained in Batf^{-/-}T cells at 62 h after stimulation (FIG. 11). Finally, microarray analysis indicated that many IL-6-induced genes were Batf-dependent (FIG. 10c and Table 2), but very few TGF-β-induced genes were Batf-dependent.

TABLE 2

| probe set | NAME of Nucleic Acid | 01_WT_{—} | 02_WT_{—} | 03_WT_{—} | 04_WT_{—} | 05_KO_{—} | 06_KO_{—} | 07_KO_{—} | 08_KO_{—} |
|---|---|---|---|---|---|---|---|---|---|
| [Cluster 8] | Sequence | Th17 | TGFb | IL-6 | neutr | Th17 | TGFb | IL-6 | neutr |
| 1418402_at | a disintegrin and | 1252.01 | 534.27 | 385.4 | 146.71 | 527.73 | 280.58 | 61.6 | 46.3 |
| | metalloproteinase domain 19 | | | | | | | | |
| | (meltrin beta) | | | | | | | | |
| 1437502_x_at | CD24a antigen | 1392.32 | 251.37 | 360.66 | 52.16 | 257.11 | 629 | 144.19 | 323.19 |
| 1422631_at | aryl-hydrocarbon receptor | 1229.47 | 482.19 | 342.1 | 61.66 | 105.34 | 339.88 | 17.84 | 104.76 |
| 1454762_at | Transcribed sequences | 137.71 | 43.33 | 51.3 | 29 | 23.7 | 48.58 | 27.73 | 26.37 |
| 1416872_at | transmembrane 4 superfamily | 1731.31 | 604.59 | 661.31 | 441.39 | 345.39 | 332.53 | 334.62 | 361.61 |
| | member 6 | | | | | | | | |
| 1448501_at | transmembrane 4 superfamily | 3068.49 | 1071.56 | 1359.61 | 888.17 | 648.4 | 568.4 | 764.86 | 694.72 |
| | member 6 | | | | | | | | |
| 1435828_at | RIKEN cDNA 2810401A20 gene | 420.45 | 16.14 | 39.68 | 12.84 | 86.92 | 23.92 | 18.94 | 12.63 |
| 1447849_s_at | avian musculoaponeurotic | 1213.54 | 34.05 | 53.53 | 14.13 | 209.21 | 37.7 | 19.07 | 24.49 |
| | fibrosarcoma (v-maf) AS42 | | | | | | | | |
| | oncogene homolog | | | | | | | | |
| 1429524_at | myosin IF | 205.85 | 17.5 | 46.18 | 12.78 | 13.05 | 17.02 | 7.9 | 9 |
| 1429525_s_at | myosin IF | 168.76 | 13.95 | 37.76 | 18.19 | 21.49 | 22.12 | 15.51 | 7.59 |
| 1421672_at | interleukin 17 | 3928.26 | 77.87 | 226.59 | 40.75 | 17.16 | 11.97 | 22.14 | 38.76 |
| 1450303_at | ventral anterior homeobox | 295.52 | 35.8 | 36.31 | 11.97 | 23.97 | 2.82 | 8.79 | 3.69 |
| | containing gene 2 | | | | | | | | |
| 1427673_a_at | sema domain, immunoglobulin | 841.65 | 469.6 | 117.17 | 728.95 | 161 | 257.6 | 348.39 | 190.96 |
| | domain (Ig), short basic | | | | | | | | |
| | domain, secreted, | | | | | | | | |
| | (semaphorin) 3E | | | | | | | | |
| 1422918_at | RIKEN cDNA 1810009J06 gene | 214.31 | 67.17 | 17.72 | 82.94 | 34.41 | 10.39 | 4.92 | 5.54 |
| 1456952_at | Transcribed sequences | 306.95 | 25.88 | 35.66 | 191.15 | 21.72 | 59.46 | 24.39 | 21.38 |
| 1459355_at | Transcribed sequences | 531.68 | 0.15 | 1199.8 | 0.57 | 836.66 | 0.71 | 0.56 | 8.04 |
| 1423607_at | lumican | 485.01 | 7.96 | 891 | 54.01 | 52.35 | 7.87 | 496.02 | 26.34 |
| 1431394_a_at | RIKEN cDNA 4921513O20 gene | 137.44 | 49.45 | 181.66 | 116.03 | 20.58 | 15.7 | 14.93 | 20.77 |
| 1452740_at | myosin heavy chain 10, non- | 1844.96 | 1586.4 | 2989.23 | 2473.54 | 160.26 | 212.17 | 420.1 | 428.25 |
| | muscle | | | | | | | | |
| 1452794_x_at | spermatogenesis associated | 65.09 | 40.44 | 169.44 | 109.38 | 22.06 | 5.07 | 13.75 | 7.08 |
| | glutamate (E)-rich protein 1, | | | | | | | | |
| | pseudogene 1 | | | | | | | | |
| 1416588_at | protein tyrosine phosphatase, | 6524.4 | 6249.96 | 6737.21 | 3167.69 | 998.73 | 2629.14 | 2060.74 | 1629.37 |
| | receptor type, N | | | | | | | | |
| 1418057_at | T-cell lymphoma invasion and | 2905.77 | 2151.33 | 3665.05 | 1930.38 | 960.78 | 902.57 | 835.98 | 919 |
| | metastasis 1 | | | | | | | | |
| 1419410_at | basic leucine zipper | 3724.56 | 1928.51 | 3329.71 | 1624.7 | 28.53 | 19.86 | 35.73 | 26.78 |
| | transcription factor, ATF-like | | | | | | | | |
| 1421207_at | leukemia inhibitory factor | 6940.37 | 1939.38 | 5812.12 | 2406.87 | 1485.29 | 3000.62 | 3867.89 | 3671.27 |
| 1421375_a_at | S100 calcium binding protein | 172.16 | 38.56 | 443.26 | 21.49 | 112.65 | 23.43 | 74.47 | 24.7 |
| | A6 (calcyclin) | | | | | | | | |
| 1442350_at | 0 day neonate skin cDNA, | 174.29 | 56.12 | 499.54 | 72.76 | 130.32 | 46.61 | 91.42 | 59.93 |
| | RIKEN full-length enriched | | | | | | | | |
| | library, clone: 4632424N07 | | | | | | | | |
| | product: unknown EST, full | | | | | | | | |
| | insert sequence | | | | | | | | |
| 1428444_at | ankyrin repeat and SOCS box- | 842.85 | 334.67 | 1870.09 | 152.4 | 129.43 | 208.55 | 489.25 | 125.6 |
| | containing protein 2 | | | | | | | | |
| 1422053_at | inhibin beta-A | 4951.52 | 1096.27 | 7519.18 | 1256.7 | 791.01 | 389.84 | 2773.24 | 551.88 |
| 1421199_at | discs, large homolog 2 | 490.11 | 47.93 | 1012.78 | 125.14 | 43.8 | 11.47 | 87.3 | 22.36 |
| | (Drosophila) | | | | | | | | |
| 1423310_at | trophoblast glycoprotein | 130.84 | 9.77 | 260.82 | 18.88 | 26.56 | 5.67 | 34.06 | 22.54 |
| 1423312_at | trophoblast glycoprotein | 128.37 | 7.39 | 288.37 | 13.03 | 26.84 | 5.51 | 28.46 | 15.38 |
| 1423311_s_at | trophoblast glycoprotein | 113.48 | 9.43 | 164.16 | 6.01 | 13.26 | 3.89 | 14.91 | 11.19 |
| 1449906_at | selectin, platelet | 410.33 | 15.43 | 667.48 | 36.35 | 128.01 | 16.24 | 119.91 | 17.41 |
| 1440173_x_at | selectin, platelet | 324.11 | 23.47 | 519.82 | 28.88 | 113.46 | 5.11 | 105.35 | 18.53 |
| 1448136_at | ectonucleotide | 141.72 | 29.64 | 144.24 | 22.16 | 19.52 | 42.9 | 22.07 | 12.3 |
| | pyrophosphatase/ | | | | | | | | |
| | phosphodiesterase 2 | | | | | | | | |
| 1455843_at | fucosyltransferase 4 | 206.69 | 66.63 | 197.75 | 50.02 | 43.12 | 22.58 | 64.78 | 19.15 |
| 1448892_at | dedicator of cytokinesis 7 | 379.86 | 104.79 | 377.77 | 142.69 | 118.64 | 75.58 | 63.89 | 90.69 |
| 1418488_s_at | ankyrin repeat domain 3 | 254.7 | 93.34 | 301.36 | 83.9 | 62.15 | 44.23 | 52.17 | 36.54 |
| 1421997_s_at | integrin alpha 3 | 890.09 | 169.65 | 1015.46 | 238.15 | 125.62 | 41.43 | 190 | 67.55 |
| 1455158_at | integrin alpha 3 | 1574.11 | 315.15 | 1937.37 | 532.62 | 263.78 | 95.04 | 298.51 | 133.14 |
| 1433509_s_at | DNA segment, Chr 6, ERATO | 924.77 | 266.53 | 1020.56 | 182.43 | 448.5 | 157.91 | 214.1 | 122.11 |
| | Doi 253, expressed | | | | | | | | |
| 1418734_at | histocompatibility 2, Q region | 508.05 | 27.21 | 650.26 | 24.54 | 199.22 | 44.55 | 32.76 | 18.09 |
| | locus 1 | | | | | | | | |
| 1452028_a_at | cadherin 23 (otocadherin) | 106.09 | 14.73 | 124.69 | 19.1 | 50.91 | 15.55 | 20.8 | 9.24 |
| 1416168_at | serine (or cysteine) proteinase | 3573.2 | 154.64 | 4602 | 179.3 | 524.07 | 56.65 | 873.78 | 94.72 |
| | inhibitor, clade F, member 1 | | | | | | | | |
| 1448562_at | uridine phosphorylase 1 | 1333.41 | 49.23 | 1557.43 | 80.35 | 279.3 | 40.22 | 326.1 | 54.94 |
| 1427535_s_at | expressed sequence AW822216 | 154.75 | 22.41 | 157 | 12.51 | 34.46 | 9.78 | 22.05 | 18.37 |
| 1440505_at | RIKEN cDNA A330045H12 gene | 902.02 | 63.61 | 935.84 | 67.52 | 293.97 | 60.78 | 147.2 | 34.36 |
| 1425137_a_at | histocompatibility 2, D region | 1774.46 | 42.38 | 1773.73 | 84.71 | 397.94 | 35.03 | 312.87 | 77.82 |
| | locus 1 | | | | | | | | |
| 1423954_at | complement component 3 | 663.95 | 24.23 | 762.36 | 32.02 | 185.98 | 14.95 | 125.6 | 32.91 |
| 1426063_a_at | GTP binding protein (gene | 1716.42 | 55.27 | 1873.08 | 78.33 | 474.86 | 35.79 | 365.54 | 52.05 |
| | overexpressed in skeletal | | | | | | | | |
| | muscle) | | | | | | | | |
| 1442383_at | Transcribed sequences | 231.59 | 33.62 | 272.39 | 31.01 | 69.04 | 15.66 | 51.33 | 11.92 |
| 1452445_at | RIKEN cDNA A230035L05 gene | 355.4 | 27.89 | 428.51 | 35.76 | 113.84 | 15.56 | 42.73 | 16.81 |
| 1429206_at | RIKEN cDNA 3110048G13 gene | 435 | 63.28 | 506.53 | 83.5 | 142.61 | 26.33 | 55.98 | 29.37 |
| 1419652_s_at | RIKEN cDNA 2610200G18 gene | 94.91 | 41.31 | 137.56 | 30.64 | 44.41 | 12.59 | 24.91 | 22.51 |
| 1421096_at | transient receptor potential | 87.15 | 18.72 | 109.57 | 22.47 | 34.95 | 7.54 | 7.04 | 20.53 |
| | cation channel, subfamily C, | | | | | | | | |
| | member 1 | | | | | | | | |
| 1428923_at | RIKEN cDNA 1600032L17 gene | 137.28 | 39.68 | 175.01 | 46.99 | 72.93 | 21.69 | 22.56 | 22.22 |
| 1418393_a_at | integrin alpha 7 | 4975.93 | 111.66 | 3723.09 | 71.12 | 2691.56 | 92.72 | 768.46 | 57.14 |
| 1422557_s_at | metallothionein 1 | 10081.47 | 1027.84 | 7085.99 | 804.93 | 6535.38 | 903.34 | 1527.44 | 801.09 |
| 1437762_at | RAB39, member RAS | 107.89 | 28.44 | 128.91 | 29.26 | 85.94 | 21.98 | 14.43 | 27.61 |
| | oncogene family | | | | | | | | |
| 1435207_at | DIX domain containing 1 | 3250.83 | 523.32 | 3278.96 | 542.9 | 2214.95 | 413.01 | 463.09 | 337 |
| 1444395_at | DIX domain containing 1 | 235.84 | 52.29 | 254.74 | 38.13 | 154.03 | 32.02 | 30.01 | 24.59 |
| 1436250_at | RIKEN cDNA 5430405G05 gene | 245.7 | 38.62 | 221.67 | 28.93 | 140.95 | 18.52 | 43.23 | 22.38 |
| 1440823_x_at | RIKEN cDNA D130058I21 gene | 393.57 | 28.56 | 380.62 | 28.72 | 224.2 | 37.61 | 56.49 | 24.37 |
| 1417600_at | solute carrier family 15 | 750.13 | 304.98 | 422.36 | 181.4 | 180.85 | 137.63 | 68.95 | 103.2 |
| | (H+/peptide transporter), | | | | | | | | |
| | member 2 | | | | | | | | |
| 1428433_at | RIKEN cDNA 1110014O20 gene | 1629.34 | 600.71 | 1254.11 | 616.5 | 440.51 | 701.79 | 266.94 | 542.57 |
| 1456022_at | RIKEN cDNA B230339E18 gene | 1123.43 | 309.34 | 593.95 | 250.88 | 178.62 | 407.85 | 155.6 | 257.24 |
| 1424863_a_at | homeodomain interacting | 398.23 | 127.12 | 237.62 | 92.86 | 70.79 | 124.17 | 73.88 | 96.42 |
| | protein kinase 2 | | | | | | | | |
| 1425983_x_at | homeodomain interacting | 325.93 | 103.38 | 172.76 | 79.31 | 49.77 | 86.63 | 72.71 | 76.27 |
| | protein kinase 2 | | | | | | | | |
| 1426181_a_at | interleukin 24 | 2087.07 | 211.42 | 1484.37 | 77.22 | 365.3 | 114.07 | 765.96 | 35.18 |
| 1445068_at | mucosa associated lymphoid | 915.03 | 72.1 | 473.22 | 219.29 | 365.09 | 98.9 | 97.34 | 122.3 |
| | tissue lymphoma translocation | | | | | | | | |
| | gene 1 | | | | | | | | |
| 1432556_a_at | RIKEN cDNA 3100002J23 gene | 304.81 | 2.55 | 124.06 | 6.06 | 68.83 | 2.77 | 10.47 | 7.78 |
| 1437056_x_at | RIKEN cDNA 1810049K24 gene | 2727.59 | 45.33 | 1049.94 | 21.46 | 552.37 | 54.28 | 299.12 | 32.46 |
| 1437090_at | hypothetical protein | 166.28 | 27.27 | 66.66 | 13.29 | 23.54 | 14.07 | 19.72 | 17.9 |
| | 4921511C16 | | | | | | | | |
| 1424671_at | pleckstrin homology domain | 1003.36 | 71.25 | 475.89 | 32.48 | 168.66 | 31.69 | 85.96 | 34.96 |
| | containing, family F (with | | | | | | | | |
| | FYVE domain) member 1 | | | | | | | | |
| 1425792_a_at | RAR-related orphan receptor | 902.3 | 167.42 | 436.52 | 69.61 | 160.07 | 80.6 | 160 | 34.45 |
| | gamma | | | | | | | | |
| 1425793_a_at | RAR-related orphan receptor | 1074.21 | 184.29 | 465.27 | 65.34 | 199.98 | 78.68 | 97.54 | 34.01 |
| | gamma | | | | | | | | |
| 1418176_at | vitamin D receptor | 361.82 | 94.87 | 250.96 | 22.66 | 50.62 | 25.44 | 50.41 | 21.47 |
| 1435500_at | RAB26, member RAS oncogene | 308.06 | 36.73 | 192.31 | 11.68 | 55.62 | 4.61 | 18.96 | 17.92 |
| | family | | | | | | | | |
| 1448471_a_at | cytotoxic T lymphocyte- | 2304.61 | 141.9 | 1301.05 | 74.76 | 473.65 | 106.44 | 195.01 | 30.47 |
| | associated protein 2 beta | | | | | | | | |
| 1416811_s_at | cytotoxic T lymphocyte- | 6191.28 | 688.63 | 3463.05 | 234.42 | 1451.77 | 331.38 | 739.69 | 81.94 |
| | associated protein 2 beta | | | | | | | | |
| 1448613_at | extracellular matrix protein 1 | 3971.63 | 439.75 | 2267.53 | 172.87 | 961.62 | 341.99 | 463.39 | 171.35 |
| 1452352_at | cytotoxic T lymphocyte- | 3048.3 | 318.87 | 1732.13 | 147.34 | 723.12 | 214.08 | 345.83 | 82.94 |
| | associated protein 2 beta | | | | | | | | |
| 1422728_at | inhibin alpha | 551.12 | 44.9 | 345.14 | 36.38 | 130.37 | 25 | 68.21 | 18.71 |
| 1428283_at | cytochrome P450, family 2, | 1989.74 | 105.94 | 1172.54 | 50.54 | 380.21 | 68.21 | 103.63 | 37.55 |
| | subfamily s, polypeptide 1 | | | | | | | | |
| 1415894_at | ectonucleotide | 218.28 | 21.71 | 148.82 | 7.81 | 17.41 | 15.06 | 41.52 | 11.52 |
| | pyrophosphatase/ | | | | | | | | |
| | phosphodiesterase 2 | | | | | | | | |
| 1460204_at | cytoplasmic tyrosine kinase, | 1632.24 | 297.81 | 966.3 | 218.58 | 281.8 | 209.75 | 264.24 | 197.17 |
| | Dscr28C related (Drosophila) | | | | | | | | |
| 1444176_at | ATPase, H+ transporting, V0 | 294.38 | 10.64 | 156.62 | 6.77 | 26.07 | 5.88 | 19.58 | 7.27 |
| | subunit D, isoform 2 | | | | | | | | |
| 1418050_at | glycosylphosphatidylinositol | 1202.44 | 60.63 | 852.17 | 52.45 | 31.99 | 17.86 | 20.26 | 19.02 |
| | specific phospholipase D1 | | | | | | | | |
| 1419331_at | cadherin 17 | 467.2 | 4.19 | 309.52 | 7.87 | 16.61 | 12.4 | 4.64 | 6 |
| 1418175_at | vitamin D receptor | 116.28 | 17.71 | 72.83 | 9.86 | 11.03 | 12.46 | 17.95 | 17.68 |
| 1420530_at | neuronal d4 domain family | 141.73 | 17.7 | 91.07 | 13.73 | 17.85 | 8.29 | 14.66 | 17.37 |
| | member | | | | | | | | |
| 1427624_s_at | interleukin 10-related T cell- | 2842.39 | 55.37 | 1805.81 | 47.95 | 121.81 | 46.32 | 276.15 | 140.15 |
| | derived inducible factor beta | | | | | | | | |
| 1436481_at | Transcribed sequences | 358.88 | 73.94 | 230.56 | 44.92 | 163.13 | 56.18 | 21.81 | 48.57 |
| 1425477_x_at | histocompatibility 2, class II | 759.58 | 131.69 | 478.22 | 57.46 | 310.75 | 48.34 | 97.57 | 29.31 |
| | antigen A, beta 1 | | | | | | | | |
| 1451721_a_at | histocompatibility 2, class II | 1075.52 | 186.51 | 700.85 | 162.09 | 432.05 | 97.93 | 144.97 | 71.93 |
| | antigen A, beta 1 | | | | | | | | |
| 1429183_at | RIKEN cDNA 1200008D14 gene | 757.83 | 109.38 | 580.28 | 97.17 | 231.39 | 40.72 | 50.9 | 52.06 |
| 1423626_at | dystonin | 510.44 | 77.79 | 406.64 | 58.24 | 112.6 | 55.18 | 94.6 | 31.74 |
| 1450699_at | selenium binding protein 1 | 2803.08 | 89.91 | 2000.99 | 54.99 | 593.09 | 36.1 | 140.06 | 15.69 |
| 1425230_at | N-acetylglutamate synthase | 211.76 | 14.07 | 173.37 | 15.4 | 34.54 | 12.09 | 13.38 | 8.71 |
| 1421739_a_at | megakaryocyte-associated | 1475.79 | 106.38 | 1136.03 | 60.16 | 302.66 | 92.97 | 89.35 | 46.15 |
| | tyrosine kinase | | | | | | | | |
| 1426399_at | RIKEN cDNA 4932416A11 gene | 1028.79 | 77.42 | 843.07 | 76.28 | 231.43 | 38.47 | 64.76 | 28.53 |
| 1418003_at | RIKEN cDNA 1190002H23 gene | 3115.42 | 830.24 | 2782.24 | 719.62 | 923.44 | 629.37 | 561.09 | 403.79 |
| 1423344_at | erythropoietin receptor | 570.53 | 159.33 | 512.53 | 190.56 | 199.69 | 112.8 | 93.38 | 103.46 |
| 1417580_s_at | selenium binding protein 1 | 3538.54 | 181.22 | 2809.38 | 128.52 | 1021.44 | 78.08 | 266.21 | 34.69 |
| 1435351_at | RIKEN cDNA 2310026E23 gene | 2059.15 | 218.86 | 1670.6 | 130.32 | 712.62 | 206.45 | 195.22 | 154.6 |
| 1437842_at | gb:BB311508 | 1683.22 | 135.05 | 1476.05 | 128.11 | 753.12 | 141.26 | 263.29 | 78.69 |
| | /DB_XREF = gi:9012213 | | | | | | | | |
| | /DB_XREF = BB311508 | | | | | | | | |
| | /CLONE = B230325J01 | | | | | | | | |
| | /FEA = EST | | | | | | | | |
| | /CNT = 14 | | | | | | | | |
| | /TID = Mm.133794.1 | | | | | | | | |
| | /TIER = Stack/STK = 14 | | | | | | | | |
| | /UG = Mm.133794 | | | | | | | | |
| | /UG_TITLE = ESTs]] | | | | | | | | |
| 1439388_s_at | gb:BB251739 | 1034.47 | 111.65 | 888.36 | 94.46 | 465.23 | 83.35 | 92.99 | 66.07 |
| | /DB_XREF = gi:8944485 | | | | | | | | |
| | /DB_XREF = BB251739 | | | | | | | | |
| | /CLONE = A730047M15 | | | | | | | | |
| | /FEA = EST | | | | | | | | |
| | /CNT = 8 | | | | | | | | |
| | /TID = Mm.3758.2 | | | | | | | | |
| | /TIER = Stack | | | | | | | | |
| | /STK = 8 | | | | | | | | |
| | /UG = Mm.3758 | | | | | | | | |
| | /LL = 12927 | | | | | | | | |
| | /UG_GENE = Crkas | | | | | | | | |
| | /UG_TITLE = v-crk-associated | | | | | | | | |
| | tyrosine kinase substrate]] | | | | | | | | |
| 1455794_at | RIKEN cDNA D130058I21 gene | 930.74 | 63.22 | 732.49 | 56.67 | 380.72 | 54.67 | 64.61 | 41.66 |
| 1449799_s_at | RIKEN cDNA 1200008D14 gene | 1100.76 | 262.35 | 1041.3 | 240.33 | 475.28 | 112.01 | 118.37 | 126.37 |
| 1452473_at | RIKEN cDNA E130201N16 gene | 146.17 | 34.12 | 113.46 | 18.37 | 59.61 | 6.77 | 10.07 | 4 |

### RORγt is Insufficient to Drive Th17 Differentiation

Since RORγt acts directly on the IL-17 promoter, we tested whether forcing RORγt expression would rescue TH17 development in Batf^{-/-}T cells. RORγt overexpression induced IL-17 production in Batf^{+/+}T cells (FIG. 10e) as expected, but failed to restore IL-17 production in Batf^{-/-}T cells (FIG. 10e and FIG. 11c). Infection with RORγt retrovirus induced 38% IL-17 production in Batf^{+/+}T cells activated under neutral conditions compared to only 1.6% IL-17 production in control retrovirus infected cells (FIG. 11c). In contrast, the RORγt retrovirus induced only 5.7% IL-17 production in Batf^{-/-} T cells under neutral conditions, and failed to restore IL-17 production even under TH17-inducing conditions (FIGS. 11b and c). Thus, in the absence of Batf, RORγt is insufficient to drive TH17 differentiation, suggesting that Batf might be required directly for transcription of IL-17 and other TH17-specific genes.

### Example 8

### DLGH2 Membrane-Associated Guanylate Kinase is Batf-Dependent

As part of our preliminary studies for this proposal, we have developed a comprehensive matrix of tissue specific transcriptional profiles to identify candidate genes important for T effector cell development. One example of a Batf-dependent gene that is induced by IL-6 in differentiating Th17 cells is DLGH2 guanylate kinase (FIG. 12). Strikingly, DLGH2−/− T cells are less efficient in Th17 differentiation (FIG. 13). In this context, we and others have reported that a subset of molecules (including DLGs) become polarized at the immune synapse in premitotic T cells and remain polarized during the T cell migratory phase. It is thought that such synapse structures likely function as a niche that organizes asymmetric partitioning of components between daughter cells at mitosis resulting in differentiation into effector cells. As an example, the protein T-bet, implicated in T helper differentiation, is differentially inherited by daughter cells in a Dlg-dependent manner. DLG kinases may regulate such T cell activation/polarization via regulation of PTEN/PI3 kinase, p38 kinase, Ca++channels and/or NFAT proteins, all of which have been identified as direct interacting partners for DLG (FIG. 14). In addition, DLG kinases bind PIPs—a mechanism for sensing/integrating PIP signaling events during cellular polarization. Thus, understanding DLGH2 in the context of Th17 development may facilitate target identification.

### Example 9

### DLGH is Required for Normal Lymphocyte Development and Activation

Among several newly discovered tumor suppressor genes, the discs large (Dlg) family represents a unique class of PDZ domain-containing membrane associated guanylate kinases (MAGUKs) that maintain cell polarity and regulate cell cycle progression. While mutations in the discs large gene lead to a loss of cell polarity and transformation of epithelial cells, very little is known about how Dlg proteins regulate lymphocyte signaling and development. We have recently reported that Dlg1 localizes to the distal pole complex in activated T cells and attenuates T cell responses (FIG. 15). Based on these findings, we hypothesized that Dlg1 regulates thymocyte signaling during development and tested this hypothesis in Dlg1f/f Lck-Cre+ mice conditionally lacking Dlg1 in T cells. By restricting TCR usage in these mice to the MHCI-restricted OT1 TCR or the MHCII-restricted OT2 TCR, we determined that Dlg1 deficiency leads to thymic atrophy (FIG. 16). Specifically, Dlg1-deficient mice exhibited relatively unperturbed numbers of double negative thymocytes with a dramatic reduction in double positive thymocytes. Thus, DLGH1 is critical for the regulation of antigen receptor signaling outputs and the regulation of proliferative responses and c-myc expression in both developing and mature lymphocytes. To the best of our knowledge, these results are the first direct evidence for a role for a dig-related MAGUK protein in lymphocytes using gene-targeted mice and indicate that DLGH1 functions as a critical negative regulator of lymphocyte proliferation, consistent with the role of the fly ortholog of the mammalian DLGH1.

### Example 10

### Batf Binds IL-17 Promoter

We tested reporter activity of the IL-17 promoter in primary Batf^{+/+} and Batf^{-/-}T cells (FIG. 17a). On day 3 of activation under TH17 conditions, Batf^{-/-} CD4+T cells showed considerably less reporter activity than Batf^{+/+}T cells, suggesting that the 1 kb proximal IL-17 promoter is Batf-responsive (FIG. 17a). To test whether this is due to direct interactions with Batf, we performed chromatin immunoprecipitation (ChIP) and elecrophoretic mobility shift assays (EMSA). For ChIP analysis, we examined a region of the IL-17 promoter (−243 to −176) and several additional highly conserved regions within the IL-17A/IL-17F locus. Batf bound to several intergenic regions as early as 24 hours, and directly to the proximal IL-17 promoter by day 5 after stimulation (FIG. 17b, FIG. 18a, b). When we tested an AP-1 consensus probe in EMSA, this probe formed two complexes in Batf^{+/+}TH17 cell extracts (FIG. 17c, lane 1). Only the upper complex formed in Batf^{-/-}TH17 extracts (FIG. 17c, lane 2), suggesting the lower complex contains Batf. Consistently, an anti-Batf antibody blocked the formation of the lower complex (FIG. 17c). Using extracts from TH17 cells derived from Batf-transgenic mice, the lower complex was more abundant compared to non-transgenic TH17 cells and was specifically supershifted by an anti-FLAG epitope antibody (FIG. 17c, lanes 7-12). Thus, of the two complexes binding the consensus AP-1 probe in TH17 cells, Batf is contained specifically within the lower complex.

### Example 11

### Identification and Characterization of a Batf Binding Site

Since Batf was required for IL-17, IL-21 and IL-22 expression (FIG. 10), we surveyed their promoters for Batf binding in a competitor-supershift assay using the AP-1 probe (FIG. 18c-e). This approach identified multiple potential Batf binding sites. First, the region in the IL-17 promoter binding Batf in ChIP assays also bound Batf by EMSA (−188 to −210) (FIG. 17b, FIG. 18c). Another region binding Batf by EMSA (−155 to −187) overlaps with a recently identified ROR-responsive element (RORE) suggested to bind RORγt, but also contains a sequence (TGACCTCA) closely resembling an AP-1 consensus element. This region (−155 to −187), but not the RORE element in the CNS-2 region of IL-17 (Ref 25), inhibited formation of both upper and lower EMSA complexes formed by the AP-1 probe (FIG. 17c, lanes 3, 4). Thus, the IL-17 promoter region between −155 and −187 interacts with complexes binding the AP-1 probe independently of its ability to bind RORs. More importantly, this element itself formed two complexes in extracts from Batf+/+TH17 cells, which were both augmented by stimulation (FIG. 17d, lanes 1-4). Again, the lower complex was selectively inhibited by an anti-Batf antibody, was absent in Batf−/− TH17 cells, and specifically supershifted by an anti-FLAG antibody (FIG. 17d). Thus, the region between −155 and −187 of the IL-17 promoter specifically contains a Batf-binding element. Finally, we identified a Batf binding motif by using the CONSENSUS program by analyzing all Batf-binding sequences in the IL-17, IL-21 and IL-22 promoters. The derived consensus logo (FIG. 17e) contains a conserved AP-1 half-site but exhibits sequence variation in the remaining nucleotides, as such differing from a consensus AP-1 response element. In summary, Batf-binding elements are distributed within the promoters of IL-17, IL-21 and IL-22 and contain a unique motif, distinct from the expected AP-1 consensus.

### Materials and Methods for Examples 1-11

Generation of Batf−/− mice. Murine Batf exons 1-2 were deleted by homologous recombination via a targeting vector constructed in pLNTK1 using a 1 kb genomic fragment (left arm) upstream of the Batf exon 1 and a 3.6 kb genomic fragment (right arm) downstream of exon 2. The left arm was generated by PCR from genomic DNA with the use of the following oligonucleotides: left arm forward (5′-ATTACTCGAGTGAAACAAACAGGCAGTCGCAGTG) (SEQ ID NO:3) and left arm reverse (5′-ATTACTCGAGCCTACTACCTTTCAGGGCTACTGC) (SEQ ID NO:4). The right arm was generated by PCR with the use of the following oligonucleotides: right arm forward (5′-ATTAGTCGACGCATTCTTCATGGTCCTTAGCCTTGG) (SEQ ID NO:5) and right arm reverse (5′-ATTAGTCGACCAGAGAATGAGAAATGTTGGAGG) (SEQ ID NO:6). EDJ22 embryonic stem cells were transfected with linearized targeting vector and targeted clones were identified by Southern analysis using probes A and B located 5′ to the left arm and 3′ to the right arm respectively. Probe A was generated using the oligonucleotides 5′-CAACTGGGTCTGAGTCAAGAGGT (SEQ ID NO:7) and 5′-CGTAGCCGCTGATTGTTTTAGAAC (SEQ ID NO:8) to generate a 531 by product. Probe B was generated using the oligonucleotides 5′-ACAGCTTGAACTTCAGAGCCCTCC (SEQ ID NO:9) and 5′-CACATTTAAGTCACAATAACACTGC (SEQ ID NO:10) to generate a 772 by product. The neomycin resistance cassette was deleted from successfully targeted clones by in vitro treatment of clones with Adeno-Cre virus and targeted clones with successful neo deletion were identified by Southern blot using probes A and B (FIGS. 1b and c). Blastocyst injections were performed with two distinct recombinant clones each of which generated germline transmission of the targeted Batf allele. Male chimeras were crossed with 129SvEv females to establish Batf mutants on the pure 129SvEv genetic background. All experiments were performed with mice harboring the neo-deleted mutant allele. Homozygous mice were obtained by intercrossing heterozygous siblings and littermates were used as controls in most experiments. For some experiments 129SvEv wild type mice purchased from Taconic served as controls. For the generation of transgenic mice, Batf cDNA was cloned from CD4⁺T cell mRNA using primers 5′-GGAAGATTAGAACCATGCCTC (SEQ ID NO:11) and 5′-AGAAGGTCAGGGCTGGAAG (SEQ ID NO:12) and subcloned into the GFP-RV retrovirus. An N-terminal FLAG tag was introduced by Quick Change Mutagenesis kit (Stratagene) using the primers 5′-GGACTACAAAGACGATGACGACAAGCCTCACAGCTCCGACAGCA (SEQ ID NO:13) and 5′-CTTGTCGTCATCGTCTTTGTAGTCCATGGTTCTAATCTTCCAGATC (SEQ ID NO:14). The underlined sequence indicates nucleotides used to introduce the FLAG-tag. The FLAG-tagged Batf was cloned into the CD2 microinjection cassette via blunt end strategy into Sma1 digested CD2 microinjection cassette. Transgene expression in CD4+T cells was tested by anti-Flag western blot. CD2-Batf transgenic mice were crossed to C57BL/6 and BALB/c mice for at least 5 generations. Transgene-negative littermates were used as control mice. Mice were bred and maintained at the animal facilities at Washington University in St. Louis. All animal experiments were approved by the Animal Studies Committee at Washington University.

Visualization of lymph nodes. To visualize superficial inguinal lymph nodes mice were injected with 50 μl of 1% Evans Blue dye solution into each hind foot pad. After 1.5 hours mice were sacrificed and lymph nodes were visualized using a dissecting microscope.

Western analysis. Total splenocytes were stimulated with anti-CD3 for three days under TH17 conditions. Cells were then lysed in RIPA buffer, electrophoresed on 15% polyacrylamide gels and transferred to nitrocellulose. Affinity purified rabbit anti-murine Batf polyclonal serum (Brookwood Biomedical; Birmingham, Ala.) was generated by immunization with full length recombinant Batf protein. Equal protein loading was assessed by subsequent immunoblotting with antibody to β-actin (Santa Cruz Biotechnology).

Isolation of dendritic cells for flow cytometry. Spleens were isolated, cut into small pieces and digested with Collagenase B (Roche) and DNase I (Sigma) for 30 min at 37° C. Red blood cells were lysed by incubation with Red Blood Cell Lysis Buffer (Sigma) (1 minute at room temperature). Single cell suspensions were prepared by passing digested spleens through 35 μm nylon cell strainers (Fisher Scientific) and were stained with antibodies for analysis by Flow Cytometry.

Isolation of naïve T cells. Splenic single cells suspensions were generated and red blood cells were lysed by incubation with Red Blood Cell Lysis Buffer (Sigma) (1 minute at room temperature). Splenocytes were then negatively depleted of B220⁺ and CD8⁺ cells using magnetically labeled beads followed by depletion over LD columns (all Miltenyi Biotec). The depleted fraction was then stained with antibodies to CD4, CD62L and CD25 (all BDPharmingen) and CD4⁺CD62L⁺CD25⁻ cells were sorted on a MoFlo cytometer. Sort purity was generally >98%. For some experiments, as indicated, CD4⁺T cells were isolated from spleens by incubation with anti-CD4 magnetic beads and selection via LS columns (Miltenyi Biotec) according to the manufacturer's recommendations.

Cell culture. For T cell differentiation assays, sorted naïve CD4⁺ CD62L⁺CD25⁻T cells were cultured at 0.5×10⁶ cells/well in 48 well plates containing plate-bound anti-CD3 (from ascites) and soluble anti-CD28 (37.5; BioXcell; 4 μg/ml). Stimulation of cells without the addition of cytokines was defined as drift condition. Cultures were supplemented with anti-IL-4 (11B11; hybridoma supernatant), IFN-γ (Peprotech; 0.1 ng/ml) and IL-12 (Genetics Institute; 10 U/ml) for TH1; anti-IFN-γ (H22; BioXcell; 10 μg/ml), anti-IL-12 (Tosh; BioXcell; 10 μg/ml) and IL-4 (Peprotech; 10 ng/ml) for TH2; anti-IL-4, anti-IL-12, anti-IFN-γ, IL-6 (Peprotech; 20 ng/ml) and TGF-β (Peprotech; 0.5 ng/ml) for TH17 differentiation. In some experiments, cultures were supplemented with IL-1β (10 ng/ml), TNFα (10 ng/ml), IL-21 (50 ng/ml; all Peprotech), anti-IL-6 (MP5-20F3; eBioscience; 10 μg/ml), anti-TGF-β (1D11, R&D Biosystems, 10 μg/ml) or anti-IL-2 (JES6-1A12; BioXcell; 10 μg/ml) as indicated. For drift, TH1 and TH2 conditions cells were restimulated on day 7 with anti-CD3 and anti-CD28. Brefeldin A was added for the last 4 hours of stimulation. For TH17 conditions, cells were restimulated on day three or day seven after activation as indicated with Phorbol 12-myristate 13-acetate (PMA) (50 ng/ml; Sigma) and ionomycin (1 μM; Sigma) for 4 hours in the presence of Brefeldin A (1 μg/ml; Epicentre). Cells were then analyzed by intracellular cytokine staining and flow cytometry.

In some experiments, as indicated, magnetically purified CD4+T cells from D011.10 transgenic mice were activated with OVA (3 μM) and irradiated splenocytes in the presence of anti-IL-4, anti-IL-12, anti-IFN-γ, IL-6 and TGF-β (1 ng/ml) to induce TH17 differentiation.

To induce TH17 differentiation in total splenocytes, single cells suspensions from spleens were prepared and red blood cells were lysed. Total splenocytes were activated at 4×10⁶ cells/well in 12 well plates containing plate-bound anti-CD3, anti-IL-4 (hybridoma supernatant), anti-IL-12 (10 μg/ml), anti-IFN-γ (10 μg/ml), IL-6 (20 ng/ml) and TGF-β (1 ng/ml). Cells were restimulated with PMA and ionomycin for 4 hours in the presence of Brefeldin A before intracellular cytokine staining and analysis by flow cytometry. For STAT3-phosphorylation assays magnetically purified CD4⁺T cells were stimulated with anti-CD3 and anti-CD28 in the presence of IL-6 or IL-21 (50 ng/ml) followed by intracellular staining and analysis by flow cytometry.

Isolation of Lamina Propria T cells. For isolation of lamina propria T cells, mice were sacrificed; small intestines removed, placed in cold DMEM media (10% FCS) and cleared of Peyer's patches and residual mesenteric fat tissue. Intestines were then opened longitudinally, cleared of contents and cut into 0.5 cm pieces. The pieces were washed multiple times in cold media and twice in ice cold Citrate BSA (CB-BSA) buffer followed by two 15 minute incubations in CB-BSA with agitation. After each incubation cells were vortexed to remove epithelial cells. The remaining intestinal pieces were then washed twice with cold media before digestion in media containing 75 U/ml Collagenase IV (Sigma) at 37° C. for 1 hour. The solution was vortexed at 20 min intervals to detach lymphocytes. After one hour the solution was filtered through a 35 μm strainer, the pieces were collected and digested a second time. Supernatants from both digestions were combined, washed once, suspended in the 70% fraction of a percoll gradient and overlaid with 37% and 30% percoll gradient fractions. Lymphocytes were collected at the 70-37% interface, washed once in PBS and stimulated with PMA/ionomycin for 3 hours before cells were stained for extracellular markers and intracellular cytokines.

Induction of EAE and disease scoring. Age and sex matched mice (7-10 weeks old) were immunized subcutaneously with 100 μg MOG35-55 peptide (Sigma) emulsified in CFA (IFA supplemented with 500 μg Mycobacterium tuberculosis) on day 0. On days 1 and 3, mice were injected with 300 ng Pertussis Toxin (List Biological Laboratories) intraperitonally (i.p.). Clinical scores were given on a scale of 1-5 as follows: 0, no overt signs of disease; 1, limp tail or hind limb weakness, but not both; 2, limp tail and hind limb weakness; 3, partial hind limb paralysis; 4, complete hind limb paralysis; 5, moribund state or death by EAE. Mice with a score of 4 were given 300 μl saline solution subcutaneously to prevent dehydration. Mice with a score of 5 were euthanized. Some mice died during the course of the experiment. Their score of 5 was included in the analysis for the remainder of the experiment. For T cell transfer experiments, CD4⁺T cells were isolated from splenic single cell suspensions by magnetic separation with anti-CD4 magnetic beads and positive selection via LS columns (Miltenyi Biotec). 1×10⁷ MACS purified CD4⁺T cells were injected i.p. on day −4 followed by EAE induction on day 0 as described above.

Isolation of CNS lymphocytes. Brain and spinal cords were removed from mice after perfusion with 30 ml of saline solution. Single cell suspensions were prepared by dispersion through sterile 35μ nylon cell strainers (Fisher Scientific) and mixed at room temperature for 1 hr in HBSS containing 0.1% collagenase, 0.1 μg/ml TLCK (N-α-tosyl-L-lysine chloromethylketone hydrochloride), and 10 μg/ml DNaseI (all Sigma). The resulting suspension was pelleted, resuspended in the 70% fraction of a Percoll gradient and overlaid by additional 37% and 30% layers. The Percoll gradient separation was achieved by centrifugation for 20 min at 2000 rpm and lymphocytes were collected at the 70-37% interface. Subsequently cells were activated with PMA and ionomycin for 3-4 hours in the presence of Brefeldin A and intracellular cytokine staining was performed.

Real time PCR. Naïve CD4⁺CD62L⁺CD25⁻T cells were isolated by cell sorting and activated with plate-bound anti-CD3 and soluble anti-CD28 antibodies under TH17 conditions for 3 days, unless otherwise indicated. Total RNA was isolated from the indicated cells using Quiagen RNeasy Mini Kit and cDNA was synthesized using SuperscriptIII reverse transcriptase (Invitrogen). Real time PCR analysis was performed using ABI SYBR Green master mix according to the manufacturer's instructions on an ABI7000 machine (Applied Biosystems) using the relative standard curve method. The PCR conditions were 2 min at 50° C., 10 min at 95° C. followed by 40 2-step cycles of 15 s at 95° C. and 1 min at 60° C. Primers for RORγt (RORγt Forward 5′-CGCTGAGAGGGCTTCAC(SEQ ID NO:15), RORγt reverse 5′-GCAGGAGTAGGCCACATTACA) (SEQ ID NO:16), IL-21 (IL-21 forward 5′-ATCCTGAACTTCTATCAGCTCCAC (SEQ ID NO:17), IL-21 reverse 5′-GCATTTAGCTATGTGCTTCTGTTTC (SEQ ID NO:18)), IL-22 (IL-22 forward-5′CATGCAGGAGGTGGTACCTT (SEQ ID NO:19), IL-22 reverse-5′-CAGACGCAAGCATTTCTCAG (SEQ ID NO:20)), RORα (RORα forward 5′-TCTCCCTGCGCTCTCCGCAC(SEQ ID NO:21), RORα reverse 5′-TCCACAGATCTTGCATGGA (SEQ ID NO:22)), IRF-4 (IRF-4 forward 5′-GCCCAACAAGCTAGAAAG (SEQ ID NO:23), IRF-4 reverse: 5′-TCTCTGAGGGTCTGGAAACT (SEQ ID NO:24)) and HPRT as normalization control (HPRT forward 5′-AGCCTAAGATGAGCGCC(SEQ ID NO:25), HPRT reverse 5′-TTACTAGGCAGATGGCCACA (SEQ ID NO:26)) were used to evaluate relative gene expression.

Gene expression profiling. Naïve CD4⁺CD62L⁺CD25⁻T cells and CD4⁺CD62L⁺CD25⁺ regulatory T cells were isolated from C57BL/6 mice. Naïve CD4⁺CD62L⁺CD25⁻T cells were differentiated under TH1 and TH2 conditions for 7 days. After restimulation with anti-CD3 and anti-CD28 for 24 hours, TH1 and TH2 cells were sorted for IFN-γ and IL-4 production respectively using cytokine secretion assays (Miltenyi Biotec) according to the Manufacturer's recommendations. For gene expression profiling of TH17 cells, naïve CD4⁺CD62L⁺CD25⁻T cells were activated for 3 days with anti-CD3 and anti-CD28 in the presence of anti-IL-4, anti-IL-12, anti-IFN-γ, anti-IL-2, IL-6 and TGF-β (0.5 ng/ml). For gene expression analysis in Batf^{-/-}T cells, naive CD4⁺CD62L⁺CD25⁻T cells from Batf^{+/+} and Batf^{-/-} mice were activated for 3 days with anti-CD3 and anti-CD28 in the presence of either anti-IL-4, anti-IL-12, anti-IFN-γ, IL-6 and TGF-β (0.5 ng/ml); anti-IL-4, anti-IL-12, anti-IFN-γ, IL-6 and anti-TGF-β; anti-IL-4, anti-IL-12, anti-IFN-γ, anti-IL-6 and TGF-β or anti-IL-4, anti-IL-12, anti-IFN-γ, anti-IL-6 and anti-TGF-β. IL-2 was neutralized in all conditions. Total RNA was isolated from cells using Quiagen Rneasy Mini Kit. Biotinylated antisense cRNA was generated using two cycle target preparation kit (Affymetrix). After fragmentation, cRNA was hybridized to Affymetrix GeneChip Mouse Genome 430 2.0 Arrays. Data were normalized and expression values were modeled using DNA-Chip analyzer (dChip) software.

Retroviral infection and analysis. mRNA was isolated from 129SvEv total thymocytes using Quiagen RNAeasy Mini Kit and cDNA was amplified by SuperscriptIII (Invitrogen). Murine RORγt transcript was amplified using primers 5′-CTCGAGGTGTGCTGTCCTGGGCTAC (SEQ ID NO:27) and 5′-CTCGAGGGGAGACGGGTCAGAGGG (SEQ ID NO:28). Underlined nucleotides indicate XhoI overhangs used to clone RORγt into XhoI digested GFP-RV2. The retrovirus based reporter hCD4-pA-GFP-RV10 has been described previously and was modified as follows to generate hCD4-pA-GFP-RV-IL-17p. The 1021 by promoter region of murine IL-17a was generated by PCR from genomic 129SvEv DNA using primers 5′-AAGCTTGAACAGGAGCTATCGGTCC (SEQ ID NO:29) and 5′-AAGCTTGAGGTGGATGAAGAGTAGTGC (SEQ ID NO:30). Underlined nucleotides indicate overhangs containing HindIII restriction sites used to clone the resulting PCR product into hCD4-pA-GFP-RV. Retroviral vectors were packaged in Phoenix E cells as described previously 2. Magnetically purified CD4⁺T cells were infected with viral supernatants on days 1 and 2 after activation with anti-CD3 and anti-CD28. Three days after activation cells were restimulated with PMA/ionomycin in the presence of Brefeldin A and analyzed by intracellular cytokine staining and Flow Cytometry. For the experiments in FIG. 4, CD4⁺T cells from Batf^{+/+} and Batf^{-/-} mice were activated under TH17 conditions, infected with the IL-17 reporter virus, and stably infected T cells were examined for GFP expression 3 days after activation.

Statistical Analysis. A Student's unpaired two-tailed t-test was used to indicate statistically significant differences between indicated groups. Differences with a P value <0.05 were considered significant.

Electrophoretic mobility shift assays. Whole cell extracts were prepared from total splenocytes activated for three days with anti-CD3, TGF-β and IL-6 as described previously. For EMSA analysis the AP-1 consensus probe, RORE element in CNS2 of the IL-17 gene8 and −187 to −155 of the IL-17 promoter (top: GGTTCTGTGCTGACCTCATTTGAGGATG (SEQ ID NO:31) and bottom: AAAAGACTGGGTGAAATTTAGTTAAAG (SEQ ID NO:32)) were used after labeling with ³²P-dCTP. The probe (2.5×10⁴ cpm per reaction) was used along with 15 μg of total cell extracts and 1 ug poly diDC as described previously. For competitor-supershift assay, Batf binding to the AP-1 consensus probe was assessed by anti-FLAG supershift. Unlabeled probes from the IL-17a, IL-21 and IL-22 promoters (Table 3) were used to compete for Batf binding to the AP-1 consensus probe. Single stranded overhangs of the competitor oligos were not filled in. Sequences identified as competitors for Batf binding were used to determine the Batf consensus motif.

TABLE 3

| | Chr. 1 | | Chr. 1 |
|---|---|---|---|
| Primers | location | 5′FAM 3′ BHQ1 Probes | location |
| IL17a −97 (−97kb) | | | |
| 5′ AAATGTGAGCCCCAGATCGA 3′ (SEQ ID | 20,623,606-20,623,625 | CTGCTGCTGTCCCAGG | 20,623,627-20,623,650 |
| NO: 33) | | CACAGTTG (SEQ ID | |
| 5′ GGGACATTTTTTCCACCATGA 3′ (SEQ ID | 20,623,652-20,623,672 | NO: 35) | |
| NO: 34) | | | |
| IL17a −60 (−60kb) | | | |
| 5′ TTGTCCCCTGGCTGTTCCT 3′ (SEQ ID | 20,661,177-20,661,247 | CCTTATCCAGCTGTCTT | 20,661.249-20,661,272 |
| NO: 36) | | TTTCTCT (SEQ ID | |
| 5′ GGGCTCCCCAAAAATTCACA 3′ (SEQ ID | 20,661,274-20,661,293 | NO: 38) | |
| NO: 37) | | | |
| IL17a −37 (−37kb) | | | |
| 5′ GTCCCTCTGTTGTTTCCAAGGAT 3′(SEQ ID | 20,683,616-20,683,638 | TCATTGAGTCCTTCCA | 20,683,640-20,683,669 |
| NO: 39) | | GCAGAGATTTCAGG(SEQ | |
| 5′ GCCATTTCAGCCACTGTGAA 3′ (SEQ ID | 20,683,671-20,683,690 | ID NO: 41) | |
| NO: 40) | | | |
| IL17a −15 (−15kb) | | | |
| 5′ TGGCAAATGTTTTGTCAACCA 3′ (SEQ ID | 20,705,507-20,705,527 | TTCCTCGATTGCTGTCT | 20,705,529-20,705,552 |
| NO: 42) | | ACTCATC (SEQ ID | |
| 5′ CATGCAGCCTCTGCTTGAGA 3′ (SEQ ID | 20,705,554-20,705,573 | NO: 44) | |
| NO: 43) | | | |
| IL17a −5 (−5kb) | | | |
| 5′ CGATACTTTTCAGTGACATCCGTTT 3′ (SEQ | 20,715,852-20,715,876 | ACTTGAAACCCAGTCA | 20,715,879-20,715,908 |
| ID NO: 45) | | GTTGCTGACCTTGA | |
| 5′ TGCTGACTTCATCTGATACCCTTAGA 3′ | 20,715,910-20,715,935 | (SEQ ID NO: 47) | |
| (SEQ ID NO: 46) | | | |
| IL17a promoter (−243 to −176) | | | |
| 5′ GAACTTCTGCCCTTCCCATCT 3′ (SEQ ID | 20,720,800-20,720,820 | CCTTCGAGACAGATGT | 20,720,822-20,720,846 |
| NO: 48) | | TGCCCGTCA (SEQ ID | |
| 5′ CAGCACAGAACCACCCCTTT 3′ (SEQ ID | 20,720,848-20,720,867 | NO: 50) | |
| NO: 49) | | | |
| IL17a +9.6 (+9.6kb) | | | |
| 5′ ATTTAGGGCACAGGTGACATGA 3′(SEQ ID | 20,730,688-20,730,709 | TGGTTCTCAAAGCATA | 20,730,711-20,730,736 |
| NO: 51) | | AACCTCATTC(SEQ ID | |
| 5′ CCACTTCCCCGACCTCACTA 3′ (SEQ ID | 20,730,738-20,730,757 | NO: 53) | |
| NO: 52) | | | |
| IL17a +23 (+23kb) | | | |
| 5′ CAAATCCGTGTGCCTTCTGTT 3′ (SEQ ID | 20,744,816-20,744,836 | CTGCAGTGAGGAAGAT | 20,744,838-20,744,866 |
| NO: 54) | | GTTTCCAATGAGG(SEQ | |
| 5′ AGGTTGACTTCGTCCCTGTGA 3′ (SEQ ID | 20,744,870-20,744,890 | ID NO: 56) | |
| NO: 55) | | | |
| IL17a +28 (+28kb) | | | |
| 5′ GTGGCCTACTTCAGGCAGATG 3′(SEQ ID | 20,749,994-20,750,014 | TGAGAAGCCAGCGTCG | 20,750,016-20,750,036 |
| NO: 57) | | GGTCC (SEQ ID NO: 59) | |
| 5′ GGAGCCGATGAGAAGCATTC 3′ (SEQ ID | 20,750,039-20,750,058 | | |
| NO: 58) | | | |
| IL17a +36 (+36kb) | | | |
| 5′ AGATAATGTATCACACAGCCCTGAAG 3′ | 20,757,551-20,757,576 | AGCCAGTGCCTTAATC | 20,757,578-20,757,600 |
| (SEQ ID NO: 60) | | CATTGGG (SEQ ID | |
| 5′ CATGGTTGTGAAGTTGGTGAGATG 3′ (SEQ | 20,757,602-20,757,625 | NO: 62) | |
| ID NO: 61) | | | |
| IL17f promoter (−408 to −340) | | | |
| 5′ACTGCATGACCCGAAAGCA 3′ | 20,774,671-20,774,688 | AACCCACACGCAGAGC | 20,774,643-20,774,669 |
| (SEQ ID NO: 63) | | ATGACAAGAG | |
| 5′ TTTAATTCCCCCACAAAGCAA 3′ (SEQ ID | 20,774,620-20,774,640 | (SEQ IDNO: 65) | |
| NO: 64) | | | |
| IL17f −7 (−7kb) | | | |
| 5′ TTCCCTTTTCTGCCTTGCA | 20,782,972-20,782,990 | ACGAAGCACAGGGCTG | 20,782,996-20,783,015 |
| 3′(SEQ ID NO: 66) | | GGCC (SEQ ID NO: 68) | |
| 5′ TGTGTAACACGCAGAGTGGAATG 3′ (SEQ ID | 20,783,017-20,783,039 | | |
| NO: 67) | | | |

CONSENSUS program for determination of Batf binding motif. Sequences of the proximal promoter regions of IL-17, IL-21, and IL-22 identified as competitors for Batf binding in the competitor-supershift EMSA assay were input into CONSENSUS version v6d14. Default program parameters were applied, except for searching the reverse complement of the input sequences (c2) and uniform background nucleotide frequencies. The program was searching potential motif lengths from 5 to 15 using the expected frequency statistic (e-value) and the optimal motif length was determined as 7. The corresponding weight matrix, with a sample size adjusted information content of 4.467, was chosen from the final cycle. The enrichment of the binding motif in the input set was verified using PATSER v3e15. Using the numerically calculated cutoff score, 38/40 of the input training sequences were identified as containing the motif.

atf Chromatin immunopreciptiation (ChIP). ChIP was performed as previously described using an affinity purified anti-Batf rabbit polyclonal antibody prepared by Brookwood Biomedical (Birmingham, Ala.). Briefly, chromatin was prepared from 1×10⁷ CD4 T cells isolated from C57BL/6 Batf^{+/+} mice stimulated under TH17 polarizing conditions with anti-CD3 (2.5 μg/ml) and syngeneic splenic feeder cells, then restimulated or not at the indicated time points with PMA (50 ng/ml) and ionomycin (750 ng/ml) for 4 h. For experiments in FIG. 18, CD4⁺T cells from Batf^{+/+} and Batf^{-/-} 129SveV mice were activated with anti-CD3/CD28 coated beads under TH17 conditions for 24 hours, then processed for ChIP analysis. Immunoprecipitations were performed with 20 μg/ml Batf-specific rabbit polyclonal antibody using the Chromatin Immunoprecipitation (ChIP) Assay Kit from Millipore (Billerica, Mass.) according to the manufacturer's recommendations. Immunoprecipitated DNA released from cross-linked proteins was quantitated by real-time PCR as previously reported, and was normalized to input DNA. All real-time PCR primers and probes are included in Table 4. The analyzed sites are denoted relative to the ATG start codons for the IL17a or IL17f gene.

TABLE 4

| | SEQ ID NO: | Sequence 5′ to 3′ |
|---|---|---|
| IL-17a promoter Oligos | | |
| 33-1-top-IL17a | 70 | GCACCCAGCACCAGCTGATCAGGACGCG |
| 33-1-bot-IL17a | 71 | GTTTGCGCGTCCTGATCAGCTGGTGCTG |
| 46-14-top-IL17a | 72 | ACGAGGCACAAGTGCACCCAGCACCAGC |
| 46-14-bot-IL17a | 73 | GATCAGCTGGTGCTGGGTGCACTTGTGC |
| 69-37-top-IL17a | 74 | GCACTACTCTTCATCCACCTCACACGAG |
| 69-37-bot-IL17a | 75 | TGTGCCTCGTGTGAGGTGGATGAAGAGT |
| 83-51-top-IL17a | 76 | AAAGAGAGAAAGGAGCACTACTCTTCAT |
| 83-51-bot-IL17a | 77 | GGTGGATGAAGAGTAGTGCTCCTTTCTC |
| 100-68-top-IL17a | 78 | GTAGTAAAACCGTATAAAAAGAGAGAAA |
| 100-68-bot-IL17a | 79 | GCTCCTTTCTCTCTTTTTATACGGTTTT |
| 119-87-top-IL17a | 80 | ACGTAAGTGACCACAGAGGTAGTAAAA |
| 119-87-bot-IL17a | 81 | TACGGTTTTACTACCTCTGTGGTCACT |
| 140-106-top-IL17a | 82 | GTCACCCCCCAACCCACTCTTGACGTAAGT |
| 140-106-bot-IL17a | 83 | TGGTCACTTACGTCAAGAGTGGGTTGGGGG |
| 159-127-top-IL17a | 84 | GAATCTTTACTCAAATGGTGTCACCCCC |
| 159-127-bot-IL17a | 85 | GGTTGGGGGGTGACACCATTTGAGTAAA |
| 169-137-top-IL17a | 86 | TTTGAGGATGGAATCTTTACTCAAATGG |
| 169-137-bot-IL17a | 87 | TGACACCATTTGAGTAAAGATTCCATCC |
| 187-155-top-IL17a | 88 | GGTTCTGTGCTGACCTCATTTGAGGATG |
| 187-155-bot-IL17a | 89 | GATTCCATCCTCAAATGAGGTCAGCACA |
| 204-172-top-IL17a | 90 | GCCCGTCATAAAGGGGTGGTTCTGTGCT |
| 204-172-bot-IL17a | 91 | AGGTCAGCACAGAACCACCCCTTTATGA |
| 215-183-top-IL17a | 92 | AGACAGATGTTGCCCGTCATAAAGGGGT |
| 215-183-bot-IL17a | 93 | GAACCACCCCTTTATGACGGGCAACATC |
| 235-203-top-IL17a | 94 | GCCCTTCCCATCTACCTTCGAGACAGAT |
| 235-203-bot-IL17a | 95 | GCAACATCTGTCTCGAAGGTAGATGGGA |
| 250-217-top-IL17a | 96 | GCATAGTGAACTTCTGCCCTTCCCATCTA |
| 250-217-bot-IL17a | 97 | GAAGGTAGATGGGAAGGGCAGAAGTTCAC |
| 266-234-top-IL17a | 98 | GAAGTCATGCTTCTTTGCATAGTGAACT |
| 266-234-bot-IL17a | 99 | GCAGAAGTTCACTATGCAAAGAAGCATG |
| 281-249-top-IL17a | 100 | CTGTTCAGCTCCCAAGAAGTCATGCTTC |
| 281-249-bot-IL17a | 101 | GCAAAGAAGCATGACTTCTTGGGAGCTG |
| 302-269-top-IL17a | 102 | CTGAATCACAGCAAAGCATCTCTGTTCAG |
| 302-269-bot-IL17a | 103 | GGGAGCTGAACAGAGATGCTTTGCTGTGA |
| 320-286-top-IL17a | 104 | GTCCATACACACATGATACTGAATCACAGC |
| 320-286-bot-IL17a | 105 | GCTTTGCTGTGATTCAGTATCATGTGTGTA |
| 334-302-top-IL17a | 106 | GCAGCTTCAGATATGTCCATACACACAT |
| 334-302-bot-IL17a | 107 | GTATCATGTGTGTATGGACATATCTGAA |
| 349-317-top-IL17a | 108 | GAGCCCAGCTCTGCAGCAGCTTCAGATA |
| 349-317-bot-IL17a | 109 | GGACATATCTGAAGCTGCTGCAGAGCTG |
| 370-337-top-IL17a | 110 | GACTCACAAACCATTACTATGGAGCCCAG |
| 370-337-bot-IL17a | 111 | CAGAGCTGGGCTCCATAGTAATGGTTTGT |
| 383-351-top-IL17a | 112 | GAGACTGTCAAGAGACTCACAAACCATT |
| 383-351-bot-IL17a | 113 | ATAGTAATGGTTTGTGAGTCTCTTGACA |
| 400-368-top-IL17a | 114 | AAAGTGTGTGTCACTAGGAGACTGTCAA |
| 400-368-bot-IL17a | 115 | GTCTCTTGACAGTCTCCTAGTGACACAC |
| 416-384-top-IL17a | 116 | GATCAAGTCAAAATTCAAAGTGTGTGTC |
| 416-384-bot-IL17a | 117 | CTAGTGACACACACTTTGAATTTTGACT |
| 433-401-top-IL17a | 118 | GGTAGAAAAGTGAGAAAGATCAAGTCAA |
| 433-401-bot-IL17a | 119 | GAATTTTGACTTGATCTTTCTCACTTTT |
| 445-413-top-IL17a | 120 | GCCAGGGAATTTGGTAGAAAAGTGAGAA |
| 445-413-bot-IL17a | 121 | GATCTTTCTCACTTTTCTACCAAATTCC |
| 464-432-top-IL17a | 122 | GGGCAAGGGATGCTCTCTAGCCAGGGAA |
| 464-432-bot-IL17a | 123 | GCAAATTCCCTGGCTAGAGAGCATCCCT |
| 476-44-top-IL17a | 124 | GTGGGTTTCTTTGGGCAAGGGATGCTCT |
| 476-44-bot-IL17a | 125 | GCTAGAGAGCATCCCTTGCCCAAAGAAA |
| 497-465-top-IL17a | 126 | GTTTACATACTAAGACATTGAGTGGGTT |
| 497-465-bot-IL17a | 127 | AAAGAAACCCACTCAATGTCTTAGTATG |
| IL-21 promoter Oligos | | |
| 33-1-top-IL21 | 128 | GTCATCAGCTCCTGGAGACTCAGTTCTG |
| 33-1-bottom-IL21 | 129 | GCCACCAGAACTGAGTCTCCAGGAGCTG |
| 55-22-top-IL21 | 130 | GTGAGAACCAGACCAAGGCCCTGTCATCA |
| 55-22-bottom-IL21 | 131 | GGAGCTGATGACAGGGCCTTGGTCTGGTT |
| 67-35-top-IL21 | 132 | AGTCAGGTTGAAGTGAGAACCAGACCAA |
| 67-35-bottom-IL21 | 133 | GGGCCTTGGTCTGGTTCTCACTTCAACC |
| 88-56-top-IL21 | 134 | TAGCGACAACCTGTGCACAGTCAGGT |
| 88-56-bottom-IL21 | 135 | GTTCAACCTGACTGTGCACAGGTTGT |
| 105-73-top-IL21 | 136 | GATGAATAAATAGGTAGCCGTAGCGACA |
| 105-73-bottom-IL21 | 137 | CAGGTTGTCGCTACGGCTACCTATTTAT |
| 120-88-top-IL21 | 138 | GGCCTCTTCTTGAGGGATGAATAAATAG |
| 120-88-bottom-IL21 | 139 | GCTACCTATTTATTCATCCCTCAAGAAG |
| 137-105-top-IL21 | 140 | CTGCAATGGGAGGGCTTGGCCTCTTCTT |
| 137-105-bottom-IL21 | 141 | GCCTCAAGAAGAGGCCAAGCCCTCCCAT |
| 150-118-top-IL21 | 142 | AAAGATTTCCAGGCTGCAATGGGAGGGC |
| 150-118-bottom-IL21 | 143 | GCCAAGCCCTCCCATTGCAGCCTGGAAA |
| 174-142-top-IL21 | 144 | GTTACTCACACTCATCCACTATACAAAG |
| 174-142-bottom-IL21 | 145 | GAAATCTTTGTATAGTGGATGAGTGTGA |
| 183-151-top-IL21 | 146 | GAAAAACGAGTTACTCACACTCATCCAC |
| 183-151-bottom-IL21 | 147 | GTATAGTGGATGAGTGTGAGTAACTCGT |
| 207-175-top-IL21 | 148 | CACGTACACCTAGCCAATGGAAAAGAAA |
| 207-175-bottom-IL21 | 149 | TCGTTTTTCTTTTCCATTGGCTAGGTGT |
| 221-189-top-IL21 | 150 | TGCCCCCACACGCACACGTACACCTAGC |
| 221-189-bottom-IL21 | 151 | CATTGGCTAGGTGTACGTGTGCGTGTGG |
| 240-208-top-IL21 | 152 | TGTGGACTCTATCCATCCCTGCCCCCAC |
| 240-208-bottom-IL21 | 153 | TGCGTGTGGGGGCAGGGATGGATAGAGT |
| 254-222-top-IL21 | 154 | GATGGGGCACATTTTGTGGACTCTATCC |
| 254-222-bottom-IL21 | 155 | GGGATGGATAGAGTCCACAAAATGTGCC |
| 266-234-top-IL21 | 156 | GTCTAAGATGCAGATGGGGCACATTTTG |
| 266-234-bottom-IL21 | 157 | GTCCACAAAATGTGCCCCATCTGCATCT |
| 279-247-top-IL21 | 158 | GTCTCTTTTTCCTGTCTAAGATGCAGAT |
| 279-247-bottom-IL21 | 159 | GCCCCATCTGCATCTTAGACAGGAAAAA |
| 304-272-top-IL21 | 160 | GCTGAAAACTGGAATTCACCCATGTGTC |
| 304-272-bottom-IL21 | 161 | AAAGAGACACATGGGTGAATTCCAGTTT |
| 314-282-top-IL21 | 162 | CTTGGTGAATGCTGAAAACTGGAATTCA |
| 314-282-bottom-IL21 | 163 | ATGGGTGAATTCCAGTTTTCAGCATTCA |
| 334-303-top-IL21 | 164 | GACACACACACACACACACACCTTGGTG |
| 334-303-bottom-IL21 | 165 | GCATTCACCAAGGTGTGTGTGTGTGTGTG |
| 361-328-top-IL21 | 166 | GCCACACACACACACACACACACACACA |
| 361-328-bottom-IL21 | 167 | GTGTGTGTGTGTGTGTGTGTGTGTGTGT |
| 383-351-top-IL21 | 168 | GAAATCTGACGGTGCCTCCTGTGCCACA |
| 383-351-bottom-IL21 | 169 | GTGTGTGTGGCACAGGAGGCACCGTCAG |
| 395-363-top-IL21 | 170 | GTTTACTTCTCTGAAATCTGACGGTGCC |
| 395-363-bottom-IL21 | 171 | CAGGAGGCACCGTCAGATTTCAGAGAAG |
| 410-378-top-IL21 | 172 | GATCAAAGTGTTTGTGTTTACTTCTCTG |
| 410-378-bottom-IL21 | 173 | GATTTCAGAGAAGTAAACACAAACACTT |
| 422-390-top-IL21 | 174 | TGCAGAGCAAAAGATCAAAGTGTTTGTG |
| 422-390-bottom-IL21 | 175 | GTAAACACAAACACTTTGATCTTTTGCT |
| 447-415-top-IL21 | 176 | GACAAACCAGGTGAGGTGCCAGGGATGC |
| 447-415-bottom-IL21 | 177 | GCTCTGCATCCCTGGCACCTCACCTGGT |
| 463-429-top-IL21 | 178 | GCCTTTATGACTGTCAGACAAACCAGGTGA |
| 463-429-bottom-IL21 | 179 | GCACCTCACCTGGTTTGTCTGACAGTCATA |
| 476-445-top-IL21 | 180 | GTCATTGCAGAAGTGCCTTTATGACTGT |
| 476-445-bottom-IL21 | 181 | GTCTGACAGTCATAAAGGCACTTCTGCA |
| 494-462-top-IL21 | 182 | GCCATGCCGCTGCTTTACTCATTGCAGA |
| 494-462-bottom-IL21 | 183 | GCACTTCTGCAATGAGTAAAGCAGCGGC |
| 509-477-top-IL21 | 184 | AAAGTTCCAATAAAGGCCATGCCGCTGC |
| 509-477-bottom-IL21 | 185 | GTAAAGCAGCGGCATGGCCTTTATTGGA |
| 525-493-top-IL21 | 186 | AGTCATCACCCCATAAAAAGTTCCAATA |
| 525-493-bottom-IL21 | 187 | GCCTTTATTGGAACTTTTTATGGGGTGA |
| 543-511-top-IL21 | 188 | GGTTCAGTCAAAAAGCATAGTCATCACC |
| 543-511-bottom-IL21 | 189 | TATGGGGTGATGACTATGCTTTTTGACT |
| 558-526-top-IL21 | 190 | AATGGAGTACAGGATGGTTCAGTCAAAA |
| 558-526-bottom-IL21 | 191 | ATGCTTTTTGACTGAACCATCCTGTACT |
| 578-546-top-IL21 | 192 | GTAACCTCTTCCATCATTGCAATGGAGT |
| 578-546-bottom-IL21 | 193 | CCTGTACTCCATTGCAATGATGGAAGAG |
| 604-573-top-IL21 | 194 | GCCCATCATTTAATTCTTCCTAAGAAG |
| 604-573-bottom-IL21 | 195 | GGTTACTTCTTAGGAAGAATTAAATGA |
| 618-586-top-IL21 | 196 | AGGTTAGAAAACTAGCCCATCATTTAAT |
| 618-586-bottom-IL21 | 197 | GAAGAATTAAATGATGGGCTAGTTTTCT |
| 639-607-top-IL21 | 198 | AGGATCTAAAATACTCTTGCTAGGTTAG |
| 639-607-bottom-IL21 | 199 | GTTTTCTAACCTAGCAAGAGTATTTTAG |
| 657-625-top-IL21 | 200 | GCACCCTTACAAAAAGATAAGGATCTAA |
| 657-625-bottom-IL21 | 201 | GTATTTTAGATCCTTATCTTTTTGTAAG |
| 678-646-top-IL21 | 202 | TGGAAGCAAATCCTATTTTAACACCCTT |
| 678-646-bottom-IL21 | 203 | TTTGTAAGGGTGTTAAAATAGGATTTGC |
| 705-672-top-IL21 | 204 | GCTATTTAAAGATACACTGGTGAAAATTG |
| 705-672-bottom-IL21 | 205 | GCTTCCAATTTTCACCAGTGTATCTTTAA |
| 718-686-top-IL21 | 206 | AGGCACCATTAGTGCTATTTAAAGATAC |
| 718-686-bottom-IL21 | 207 | CCAGTGTATCTTTAAATAGCACTAATGG |
| 736-704-top-IL21 | 208 | GTTACATAAAGTGTCAGGAGGCACCATT |
| 736-704-bottom-IL21 | 209 | GCACTAATGGTGCCTCCTGACACTTTAT |
| 754-722-top-IL21 | 210 | GTATTTACAATCCATATTGTTACATAAA |
| 754-722-bottom-IL21 | 211 | GACACTTTATGTAACAATATGGATTGTA |
| 775-743-top-IL21 | 212 | AGTTCATCAAAACTGTTTATTGTATTTA |
| 775-743-bottom-IL21 | 213 | GATTGTAAATACAATAAACAGTTTTGAT |
| 792-760-top-IL21 | 214 | GAGCACGCTGTCTACTTAGTTCATCAAA |
| IL-22 promoter oligos | | |
| 33-1-top-IL22 | 215 | AGTTATCAACTGTTGACACTTGTGCGAT |
| 33-1-bottom-IL22 | 216 | CAGAGATCGCACAAGTGTCAACAGTTGA |
| 48-16-top-IL22 | 217 | ACAGGCTCTCCTCTCAGTTATCAACTGT |
| 48-16-bottom-IL22 | 218 | TGTCAACAGTTGATAACTGAGAGGAGAG |
| 69-37-top-IL22 | 219 | TTGCCTTTTGCTCTCTCACTAACAGGCT |
| 69-37-bottom-IL22 | 220 | AGGAGAGCCTGTTAGTGAGAGAGCAAAA |
| 85-53-top-IL22 | 221 | TGCTCCCCTGATGTTTTTGCCTTTTGCT |
| 85-53-bottom-IL22 | 222 | GAGAGAGCAAAAGGCAAAAACATCAGGG |
| 107-75-top-IL22 | 223 | GTACCATGCTACCCGACGAACATGCTCC |
| 107-75-bottom-IL22 | 224 | TCAGGGGAGCATGTTCGTCGGGTAGCAT |
| 123-91-top-IL22 | 225 | GACAATCATCTGCTTGGTACCATGCTAC |
| 123-91-bottom-IL22 | 226 | GTCGGGTAGCATGGTACCAAGCAGATGA |
| 146-114-top-IL22 | 227 | AGGTAAGCACTCAGACCTCTACAGACAA |
| 146-114-bottom-IL22 | 228 | GATGATTGTCTGTAGAGGTCTGAGTGCT |
| 160-128-top-IL22 | 229 | AGAGACACCTAAACAGGTAAGCACTCAG |
| 160-128-bottom-IL22 | 230 | GAGGTCTGAGTGCTTACCTGTTTAGGTG |
| 181-149-top-IL22 | 231 | TCTGCCTCTCCCATCACAAGCAGAGACA |
| 181-149-bottom-IL22 | 232 | TTAGGTGTCTCTGCTTGTGATGGGAGAG |
| 193-161-top-IL22 | 233 | AAAAGCAGCAACTTCTGCCTCTCCCATC |
| 193-161-bottom-IL22 | 234 | CTTGTGATGGGAGAGGCAGAAGTTGCTG |
| 214-182-top-IL22 | 235 | CCTGGTGTCCCGATGGCTATAAAAGCAG |
| 214-182-bottom-IL22 | 236 | AGTTGCTGCTTTTATAGCCATCGGGACA |
| 233-201-top-IL22 | 237 | GTCACAATACCAAAAAAACCCTGGTGTC |
| 233-201-bottom-IL22 | 238 | ATCGGGACACCAGGGTTTTTTTGGTATT |
| 252-220-top-IL22 | 239 | AATGTCTGATGTCATATCATTCACAATA |
| 252-220-bottom-IL22 | 240 | TTTGGTATTGTGAATGATATGACATCAG |
| 267-235-top-IL22 | 241 | GACTGGAAATTAGATAATGTCTGATGTC |
| 267-235-bottom-IL22 | 242 | GATATGACATCAGACATTATCTAATTTC |
| 293-261-top-IL22 | 243 | GTGGTTAGGTACTTCTCAGAAGACAGGA |
| 293-261-bottom-IL22 | 244 | TCCAGTCCTGTCTTCTGAGAAGTACCTA |
| 305-273-top-IL22 | 245 | TGGCCTCCTATGGTGGTTAGGTACTTCT |
| 305-273-bottom-IL22 | 246 | TTCTGAGAAGTACCTAACCACCATAGGA |
| 329-297-top-IL22 | 247 | GGAAGGCTTGGAGGTGGTGTCTTGTGGC |
| 329-297-bottom-IL22 | 248 | AGGAGGCCACAAGACACCACCTCCAAGC |
| 340-309-top-IL22 | 249 | GCTCTCAAGGTGGGAAGGCTTGGAGGTG |
| 340-309-bottom-IL22 | 250 | GACACCACCTCCAAGCCTTCCCACCTTG |
| 366-334-top-IL22 | 251 | GTGACGTTTTAGGGAAGACTTCCCATCT |
| 366-334-bottom-IL22 | 252 | TTGAGAGATGGGAAGTCTTCCCTAAAAC |
| 380-348-top-IL22 | 253 | TGTTGGCCCTCACCGTGACGTTTTAGGG |
| 380-348-bottom-IL22 | 254 | GTCTTCCCTAAAACGTCACGGTGAGGGC |
| 405-373-top-IL22 | 255 | CTGGGATTTGTGTGCAAAAGCACCTTGT |
| 405-373-bottom-IL22 | 256 | GGCCAACAAGGTGCTTTTGCACACAAAT |
| 420-388-top-IL22 | 257 | GTGTTTAGAAGATTTCTGGGATTTGTGT |
| 420-388-bottom-IL22 | 258 | TTTGCACACAAATCCCAGAAATCTTCTA |
| 497-465-top-IL22 | 259 | AATAGCTACGGGAGATCAAAGGCTGCTC |
| 497-465-bottom-IL22 | 260 | GAGTAGAGCAGCCTTTGATCTCCCGTAG |
| 518-486-top-IL22 | 261 | CCGTGACCAAAACGCTGACTCAATAGCT |
| 518-486-bottom-IL22 | 262 | CCCGTAGCTATTGAGTCAGCGTTTTGGT |
| 528-495-top-IL22 | 263 | GAAAATGAGTCCGTGACCAAAACGCTGAC |
| 528-495-bottom-IL22 | 264 | ATTGAGTCAGCGTTTTGGTCACGGACTCA |
| 536-504-top-IL22 | 265 | GTTGGTGGGAAAATGAGTCCGTGACCAA |
| 536-504-bottom-IL22 | 266 | GCGTTTTGGTCACGGACTCATTTTCCCA |
| 540-506-top-IL22 | 267 | TGAAGTTGGTGGGAAAATGAGTCCGTGACC |
| 540-506-bottom-IL22 | 268 | GTTTTGGTCACGGACTCATTTTCCCACCAA |
| 547-513-top-IL22 | 269 | GAATCTATGAAGTTGGTGGGAAAATGAGTC |
| 547-513-bottom-IL22 | 270 | TCACGGACTCATTTTCCCACCAACTTCATA |
| 558-527-top-IL22 | 271 | TAAAGAGATAAGAATCTATGAAGTTGGT |
| 558-527-bottom-IL22 | 272 | GTCCCACCAACTTCATAGATTCTTATCT |
| 574-543-top-IL22 | 273 | GTATTTCTGGTCACTTCTAAAGAGATAA |
| 574-543-bottom-IL22 | 274 | GATTCTTATCTCTTTAGAAGTGACCAGA |
| 595-563-top-IL22 | 275 | GAATATAGGACACGGGTCTTTTATTTCT |
| 595-563-bottom-IL22 | 276 | TGACCAGAAATAAAAGACCCGTGTCCTA |
| 612-580-top-IL22 | 277 | GCTTATTTCAAAGCACAGAATATAGGAC |
| 612-580-bottom-IL22 | 278 | CCCGTGTCCTATATTCTGTGCTTTGAAA |
| 628-596-top-IL22 | 279 | CCAAGTTTTCATTATGGCTTATTTCAAA |
| 628-596-bottom-IL22 | 280 | TGTGCTTTGAAATAAGCCATAATGAAAA |
| 650-619-top-IL22 | 281 | GATTTTAAAAATTGAAATAATCTCCAAG |
| 650-619-bottom-IL22 | 282 | GAAAACTTGGAGATTATTTCAATTTTTA |
| 662-630-top-IL22 | 283 | AGAGATATAATTATTTTAAAAATTGAAA |
| 662-630-bottom-IL22 | 284 | GATTATTTCAATTTTTAAAATAATTATA |
| 684-652-top-IL22 | 285 | GGATTCCATATACTAAAAAAATAGAGATA |
| 684-652-bottom-IL22 | 286 | GATTATATCTCTATTTTTTTAGTATATGG |
| 700-668-top-IL22 | 287 | AGCTAGTTATAGTTTAGGATTCCATATA |
| 700-668-bottom-IL22 | 288 | TTTAGTATATGGAATCCTAAACTATAAC |

### Example 12

### Human Batf Functions in Human Th17 Cells

The role of Batf in human Th17 cells has been analyzed. Over-expression of human Batf in human cord blood derived Th17 cells showed a 2 fold increase in IL-17 production, indicating that it augments Th17 differentiation in human cells (FIG. 19). An siRNA knockdown approach may show whether Batf is necessary for Th17 development. The feasibility of this approach was demonstrated by the siRNA mediated knockdown of RORγt with a subsequent decrease in IL-17 production (FIG. 20).

### Example 13

### The Batf Homolog Batf3 can Replace Batf in Th17 Development

We have also initiated studies to determine Batfs molecular mechanism in Th17 development. One approach for this is to compare Batf to the closely related AP1 family member Batf3 with which it has 48% sequence identity. (FIG. 21). Batf3^{-/-} mice have a phenotype distinct from that of Batf^{-/-} mice, showing normal Th17 development but lack of development of CD8α⁺ conventional dendritic cells (cDCs).

Surprisingly, retroviral reconstitution with either Batf or Batf3 restored IL-17 production in Batf/Batf3 double deficient T cells (FIG. 22) and restored CD8α⁺cDC differentiation from Batf−/− Batf3−/− bone marrow (data not shown). Thus, Batf and Batf3 are functionally equivalent when expressed at sufficient levels. However, we have not excluded the possibility that Batf has an as yet undiscovered function that Batf3 cannot fulfill. The fact that Batf is almost completely conserved between mouse and human whereas Batf and Batf3 diverge suggests an evolutionary advantage to maintaining the sequence of Batf and thus an important unique function.

Interestingly, Batf3 is expressed both in wild type and Batf^{-/-}Th17 cells and is also highly expressed in Th1 cells, as is Batf (FIG. 23). Yet, endogenous levels of Batf3 are apparently not sufficient for Th17 differentiation in Batf^{-/-}T cells. Endogenous Batf3 is also not responsible for the initial burst of RORγt expression in Batf^{-/-}T cells since early RORγt expression still occurred in Batf^{-/-}Batf3^{-/-}T cells (data not shown). Furthermore, the expression of Batf and Batf3 in Th1 cells implies that there may be a mechanism to prevent them from promoting IL-17 production in Th1 cells. Similar to mouse Batf (FIG. 23), the expression of human Batf is not restricted to Th17 cells, but is expressed in other T helper subsets as well (FIG. 24), suggesting that common mechanisms may exist in humans.

### Example 14

### Batf May be Negatively Regulated by Serine Phosphorylation

Serine phosphorylation of Batf may be important in regulating its activity. Phosphorylation of a serine residue within the DNA binding domain of Batf (S43) was suggested to inhibit Batf binding to DNA and potentially act as a dominant negative by sequestering Jun binding partners. This serine is conserved between Batf and Batf3 (FIG. 21). Mutation of Batf S43 to aspartate (S43D) to mimic phosphorylation abrogated its ability to restore IL-17 production in Batf^{-/-}Batf3^{-/-}T cells (FIG. 22). However, Batf with an S43 to alanine mutation (S43A) and several other mutations, could still restore IL-17 production (FIG. 25). This suggests that a potential mechanism for inhibiting IL-17 production in Th1 cells may be through serine phosphorylation of Batf and Batf3, both of which are highly expressed in Th1 cells (FIG. 24). We are in the process of generating monoclonal antibodies against Batf that will be valuable for analysis of its modifications. Finally, It has been recognized that Th17 cells are convertible to a Th1 or Th2 phenotype, which may be a mechanism for preventing prolonged IL-17 responses and for rapidly responding to a changing pathogenic environment. Failure to prevent serine phosphorylation of Batf in Th17s may be one mechanism that allows conversion of Th17s to Th1s and Th2s.

### Example 15

### Functions of Batf and Batf3

Both Batf and Batf3 restore IL4 induced IgG1 switching in Batf^{-/-}/Batf3^{-/-} double knockout B cells (FIG. 26). In addition, both Batf and Batf3 restore IL4 induced Th17 differentiation in Batf^{-/-}Batf3^{-/-} double knockout T cells (FIG. 27). This is in contrast to the inability of other bzip proteins such as ATF3, cFos and cMaf, to restore IL-17 production (FIG. 28).

### Example 16

### Transgenic Batf Prolongs the Ability to Produce IL-17

Transgenic Batf prolongs the ability of Th17 cells to produce IL-17. Unlike wild-type, transgenic Batf cells were capable of producing IL-17 at day ten (FIG. 29).

What is claimed is:

1. A method of modulating an immune response, the method comprising modulating Th17 cell development.

2. The method of claim 1, wherein the immune response is selected from the group consisting of an autoimmune response, an immune response against a pathogen, and an anti-tumor response.

3. The method of claim 1, wherein Th17 cell development is reduced.

4. The method of claim 1, wherein Th17 cell development is modulated by modulating Batf activity.

5. The method of claim 3, wherein Batf expression is inhibited.

6. The method of claim 2, wherein the immune response is an autoimmune response, and modulation results in inducing development of Th17 cells.

7. The method of claim 2, wherein the autoimmune response is a response against a transplanted organ.

8. The method of claim 9, wherein the immune response is an immune response against a pathogen, and modulation results in inducing development of Th17 cells.

9. The method of claim 1, wherein the immune response is modulated in a human.

10. A method of modulating Th17 cell development, the method comprising modulating Batf expression.

11. The method of claim 10, wherein Batf expression is modulated by modulating the amount of Batf or modulating the ability of Batf to bind to a Batf binding site.

12. The method of claim 11, wherein the ability of Batf to bind to a Batf binding site is modulated by modulating the phosphorylation of Batf.

13. The method of claim 10, wherein human Batf expression is modulated.

14. An isolated nucleic acid comprising a Batf binding site.

15. The isolated nucleic acid of claim 13, wherein the nucleic acid sequence is selected from a sequence of Table A.

16. The isolated nucleic acid of claim 13, wherein the nucleic acid is operably linked to a promoter nucleic acid sequence.

17. The isolated nucleic acid of claim 13, wherein the nucleic acid is operably linked to a reporter nucleic acid sequence.

18. The isolated nucleic acid of claim 13, wherein the nucleic acid is incorporated into a vector.

19. The isolated nucleic acid of claim 17, wherein the vector is incorporated into a cell.
